(19) United States
(12) Patent Application Publication
(10) Pub. No.: US 2026/0130872 A1
(43) Pub. Date: May 14, 2026

(54) Substituted Bicyclic Compounds, Compositions and Methods of Use

(71) Applicant: Deep Apple Therapeutics, Inc., South San Francisco, CA (US)

(72) Inventors: Craig Stivala, San Mateo, CA (US); Jason R. Zbieg, Montara, CA (US); Liang Zhao, San Mateo, CA (US); Stefan Gahbauer, South San Francisco, CA (US); Roger Benjamin Ruggeri, Waterford, CT (US)

(73) Assignee: Deep Apple Therapeutics, Inc., South San Francisco, CA (US)

(21) Appl. No.: 19/381,151

(22) Filed: Nov. 6, 2025

Related U.S. Application Data
(60) Provisional application No. 63/718,133, filed on Nov. 8, 2024.

(51) Int. Cl. A61K 31/17; A61K 31/167; C07C 237/20; C07C 317/28; C07C 321/28

(52) U.S. Cl. CPC A61K 31/17; A61K 31/167; C07C 237/20; C07C 317/28; C07C 321/28; C07C 2602/08

(57) ABSTRACT

The present disclosure provides compounds that are useful for the treatment of conditions mediated by GIPR. Also provided are pharmaceutical compositions containing such compounds, and methods of treatment using such compounds.

### CROSS REFERENCE TO RELATED APPLICATIONS

This application claims priority to U.S. Provisional Application No. 63/718,133, filed Nov. 8, 2024, the contents of which is incorporated herein by reference in its entirety.

### FIELD OF INVENTION

The present disclosure provides compounds that modulate GIPR and are therefore useful for the treatment of conditions, diseases and/or disorders mediated by GIPR. Also provided are pharmaceutical compositions containing such compounds and processes for preparing such.

### BACKGROUND

People are typically classified as obese when their body mass index (BMI)—a person's weight divided by the square of the person's height—is over 30 kg/m²; the range 25-30 kg/m² is defined as overweight. Obesity is a major cause of disability and is correlated with various diseases and conditions particularly cardiovascular diseases, type 2 diabetes, obstructive sleep apnea, and certain types of cancer and osteoarthritis.

Obesity is a leading preventable cause of death worldwide, with increasing rates in adults and children. In 2022, over 1 billion people were obese worldwide (879 million adults and 159 million children), representing more than a double of adult cases (and four times higher than cases among children) registered in 1990. Obesity is more common in women than in men. The World Health Organization and medical societies, e.g. the American Medical Association classify obesity as a disease.

The GBD [Global Burden of Disease] investigators found an increase in the burden of elevated BMI, with high BMI accounting for 4.0 million deaths in 2015, more than two-thirds of which were caused by cardiovascular disease (CVD), 16 even after accounting for smoking and ill health. Furthermore, a large proportion of both BMI-related deaths (41%) and BMI-related disability-adjusted life-years (34%) were caused by CVD among individuals with obesity. Circulation 2021 143:e984-e1010.

Obesity is also a risk factor for several major cancers, including post-menopausal breast, colorectal, endometrial, kidney, esophageal, pancreatic, liver, and gallbladder cancer. Excess body fat results in an approximately 17% increased risk of cancer-specific mortality. Cancers 2023, 15,485.

Moreover, the trends in obesity prevalence in the United States and around the world highlight the significant impact that obesity will continue to have on CVD incidence, cancer, Type 2 diabetes and other associated disease/conditions.

Recently GLP-1 agents have had considerable success in the treatment of obesity. Yet, many individuals on GLP-1 therapy experience adverse side effects including diarrhea, nausea and vomiting. In addition, GLP-1 agents may not sufficiently reduce fat body mass vs. lean body mass which may be aided thru the role of GIPR signaling on fat cells. Further, GLP-1 agents action on weight loss may diminish with time.

Glucose-dependent insulinotropic polypeptide (GIP) is a single 42-amino acid peptide secreted from K-cells in the small intestine (duodenum and jejunum). Human GIP is derived from the processing of proGIP, 153-amino acid precursor that is encoded by a gene localized to chromosome 17q (Inagaki et al., Endocrinol 1989; 3:1014-1021; Fehmann et al. Endocr Rev. 1995; 16:390-410). GIP was formerly called gastric inhibitory polypeptide.

GIP secretion is induced by food ingestion. GIP has a number of physiological effects in tissues, including promotion of fat storage in adipocytes and promotion of pancreatic islet-cell function and glucose-dependent insulin secretion. GIP and glucagon like polypeptide-I (GLP-1) are known insulinotropic factors (“incretins”). Intact GIP is rapidly degraded by DPPIV to an inactive form. The insulinotropic effect of GIP is lost in type 2 diabetic patients while GLP-1 's incretin effect remains intact (Nauck et al. J. Cline. Invest. 1993; 91:301-307).

The GIP receptor (GIPR) is a member of the secretin-glucagon family of class B G-protein coupled receptors (GPCRs) having an extracellular N-terminus, seven transmembrane domains and an intracellular C-terminus. The N-terminal extracellular domains of this family of receptors are usually glycosylated and form the recognition and binding domain of the receptor. GIPR is highly expressed in a number of tissues, including the pancreas, gut, adipose tissue, heart, pituitary, adrenal cortex, and brain (Udin et al., Endocrinology. 1993, 133:2861-2870). Human GIPR comprises 466 amino acids and is encoded by a gene located on chromosome 19q13.3 (Gremlich et al., Diabetes. 1995; 44:1202-8; Volz et al., FEBS Lett. 1995, 373:23-29). Studies have suggested that alternative mRNA splicing results in the production of GIP receptor variants of differing lengths in human, rat and mouse.

GIPR knockout mice (Gipr_1_) are resistant to high fat diet-induced weight gain and have improved insulin sensitivity and lipid profiles. (Yamada et al., Diabetes. 2006, 55:S86; Miyawaki et al. Nature Med. 2002, 8:738-742). In addition, a novel small molecule GIPR antagonist SKL-14959 prevents obesity and insulin resistance. (Diabetologia 2008, 51:S373, 44th EASD Annual meeting poster). SKL-14959 polypeptide suppressed body-weight gain in Mice (“Gastric inhibitory polypeptide receptor antagonist, SKL-14959, suppressed body weight gain on diet-induced obesity mice” T. Nakamura et al. Obesity Science and Practice January 2018.

There have been additional efforts to discover GIPR agents to aid weight loss. Expert Opinion on Drug Discovery 2023, VOL. 18, NO. 6, 659-669. GIPR antagonist small molecules have been disclosed e.g., WO2009/148004; EP1283058. WO2024/214038 also discloses GIPR antagonist small molecules.

Separately, WO2006/104826 discloses glucagon receptor substituted aryl and heteroaryl derivatives as antagonists for the treatment of diabetes and related conditions.

Accordingly, these links to obesity and insulin resistance imply GIPR modulation is a useful approach for therapeutic intervention. While GLP-1 agents have been beneficial in treating some obese patients, given the prevalence of obesity in modern society and its adverse health effects there is a continuing need for new agents (e.g., GIPR antagonists) that are useful for the treatment of obesity.

### SUMMARY

The present invention relates to compounds of Formula (I), as described herein including stereochemical isomeric forms thereof and pharmaceutically acceptable salts thereof, which are useful as GIPR antagonists and/or treating or preventing GIPR mediated conditions and diseases.

More specifically, in some embodiments, provided herein are compounds of Formula (I) or a pharmaceutically acceptable salt thereof:

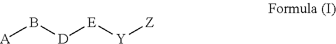

wherein
A is —C(O)OR² or —S(O)₂OR²;
R² is H or (C₁-C₆)alkyl;
B is (C₁-C₄)alkylene;
D is NH, CH₂, or is absent;
E is carbonyl, sulfonyl, NH, CH₂ or is absent;
Y is

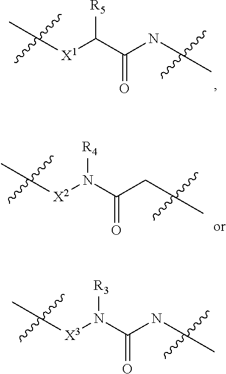

wherein
when Y is

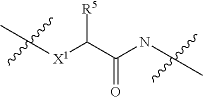

X¹ is a bicyclic ring comprising a 5-6 membered aryl ring, optionally having 0 to 3 heteroatoms selected independently from nitrogen, oxygen and sulfur, fused to a (C₅-C₇)cycloalkyl ring optionally containing 0 to 3 heteroatoms independently selected from nitrogen, oxygen and sulfur;
X¹ is optionally mono-, di- or tri-substituted independently with (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, difluoromethoxy or trifluoromethoxy;
R⁵ is (C₅-C₆)alkyl, phenyl, hetero(C₅-C₆)aryl, (C₃-C₇)cycloalkyl, (C₃-C₇)cycloalkenyl or hetero(C₃-C₇)cycloalkyl; said hetero(C₅-C₆)aryl and hetero(C₃-C₇)cycloalkyl each independently having 1 to 3 heteroatoms selected independently from nitrogen, oxygen and sulfur;
wherein said R⁵ is optionally mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, pentafluoropropanyl, mono-N— or di-N,N—(C₁-C₆)alkylaminocarbonyl, mono-N— or di-N,N—(C₁-C₆)alkylaminocarbonyloxy, (C₁-C₆)alkylcarbonyloxy, (C₁-C₆)alkylcarbonyl or (C₁-C₆)alkoxycarbonyl;
or R⁵ forms a 5-7 membered heteroaryl or heteroalkyl ring with nitrogen, optionally said heteroaryl or heteroalkyl ring each independently having one or two additional heteroatoms selected independently from nitrogen, sulfur and oxygen having the Formula X, wherein said 5-7 membered ring formed with nitrogen is optionally substituted with (C₁-C₄)alkyl, or is mono- or di-substituted independently with Tl;

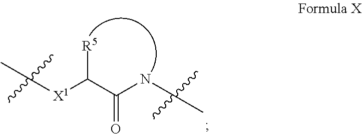

wherein T¹ is phenoxy, hetero(C₅-C₆)aryloxy, (C₃-C₇)cycloalkoxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl;
wherein said hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said T¹ is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; and
Z is Z¹; and Z¹ is phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryl or hetero(C₃-C₇)cycloalkyl each independently have 1 to 3 heteroatoms selected independently from oxygen, nitrogen and sulfur;
wherein said Z¹ is mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, (C₃-C₆)cycloalkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; or wherein said Z¹ is mono- or di-substituted independently with Q¹;
wherein Q¹ is phenoxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl;
wherein hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said Q¹ is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl;
or Z is Z¹ and Z¹ forms a 5-7 membered heteroaryl or heteroalkyl ring with R⁵, optionally said heteroaryl or heteroalkyl each independently having one or two additional heteroatoms selected independently from nitrogen, sulfur and oxygen having the Formula XI:

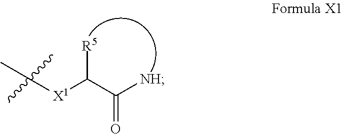

or
when Y is

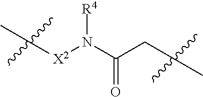

X² is a bicyclic ring comprising a 5-6 membered aryl ring optionally having 0 to 3 heteroatoms selected independently from nitrogen, oxygen and sulfur fused to a (C₅-C₇)cycloalkyl ring optionally containing 0 to 3 heteroatoms independently selected from nitrogen, oxygen and sulfur;
X² is optionally mono-, di- or tri-substituted with (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, difluoromethoxy or trifluoromethoxy;
R⁴ is (C₅-C₆)alkyl, phenyl, hetero(C₅-C₆)aryl, (C₃-C₇)cycloalkyl, (C₃-C₇)cycloalkenyl or hetero(C₃-C₇)cycloalkyl; said hetero(C₅-C₆)aryl and hetero(C₃-C₇)cycloalkyl each independently having 1 to 3 heteroatoms selected independently from nitrogen, oxygen and sulfur;
wherein said R⁴ is optionally mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, pentafluoropropanyl, mono-N— or di-N,N—(C₁-C₆)alkylaminocarbonyl, mono-N— or di-N,N—(C₁-C₆)alkylaminocarbonyloxy, (C₁-C₆)alkylcarbonyloxy, (C₁-C₆)alkylcarbonyl or (C₁-C₆)alkoxycarbonyl;
or R⁴ forms a 5-7 membered heteroaryl or heteroalkyl ring with nitrogen, said heteroaryl or heteroalkyl ring each optionally having one or two additional heteroatoms selected independently from nitrogen, sulfur and oxygen having the Formula X2; wherein said ring formed with nitrogen is optionally substituted with (C₁-C₄)alkyl; or wherein said R⁴ is mono- or di-substituted independently with T²;

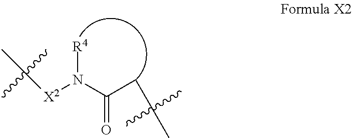

wherein T² is phenoxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said T² is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; and
Z is Z²; and Z² is phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryl or hetero(C₃-C₇)cycloalkyl each independently have 1 to 3 heteroatoms selected independently from oxygen, nitrogen and sulfur;
wherein said Z² is mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkoxy(C₁-C₆)alkyl, difluoromethyl, trifluoromethyl, trifluoroethyl, trifluoromethylthio, pentafluoroethanyl, or pentafluoropropanyl; or Z² is mono- or di-substituted independently with Q²
wherein Q² is phenoxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said Q² is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl;
or Z is Z²; and Z² forms a 5-7 membered heteroaryl or heteroalkyl ring with R⁴, optionally said heteroaryl or heteroalkyl each independently having one or two additional heteroatoms selected independently from nitrogen, sulfur and oxygen having the formula X3

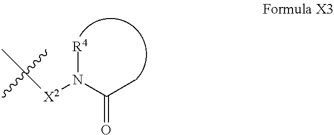

or
when Y is

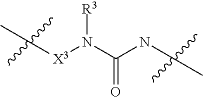

X³ is a bicyclic ring comprising a 5-6 membered aryl ring optionally having 0-3 heteroatoms selected independently from nitrogen, oxygen and sulfur fused to a (C₅-C₇)cycloalkyl ring optionally containing 0 to 3 heteroatoms independently selected from nitrogen, oxygen and sulfur;
X³ is optionally mono-, di- or tri-substituted with (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, difluoromethoxy or trifluoromethoxy;
R³ is (C₅-C₆)alkyl, phenyl, hetero(C₅-C₆)aryl, (C₃-C₇)cycloalkyl, (C₃-C₇)cycloalkenyl or hetero(C₃-C₇)cycloalkyl; said hetero(C₅-C₆)aryl and hetero(C₃-C₇)cycloalkyl each independently having 1 to 3 heteroatoms selected independently from nitrogen, oxygen and sulfur;
wherein said R³ is optionally mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, pentafluoropropanyl, mono-N— or di-N,N—(C₁-C₆)alkylaminocarbonyl, mono-N— or di-N,N—(C₁-C₆)alkylaminocarbonyloxy, (C₁-C₆)alkylcarbonyloxy, (C₁-C₆)alkylcarbonyl or (C₁-C₆)alkoxycarbonyl;
or R³ forms a 5-7 membered heteroaryl or heteroalkyl ring with the nitrogens, said heteroaryl or heteroalkyl ring each optionally having one or two additional heteroatoms selected independently from nitrogen, sulfur and oxygen having the formula X4 wherein said ring formed with nitrogens is optionally substituted with (C₁-C₄)alkyl; or wherein said R³ is mono- or di-substituted independently with T³;

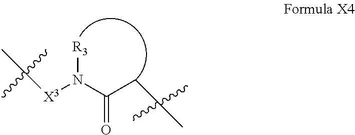

wherein T³ is phenyloxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryloxy, hetero(C₃—C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said T³ is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; and
Z is Z³; and Z³ is phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryl or hetero(C₃-C₇)cycloalkyl each independently have 1 to 3 heteroatoms selected independently from oxygen, nitrogen and sulfur;
wherein said Z³ is mono-, di- or tri-substituted independently with cyano, cyanomethyl, nitro, amino, thio, mono-N— or di-N,N—(C₁-C₆)alkylamino, mono-N— or di-N,N—(C₁-C₆)alkylamino(C₁-C₆)alkyl, (C₃-C₆)cycloalkyl, (C₁-C₆)alkoxy(C₁-C₆)alkyl, difluoromethyl, trifluoroethyl, trifluoromethylthio, pentafluoroethanyl, or pentafluoropropanyl; or Z³ is mono- or di-substituted independently with Q³;
wherein Q³ is phenyloxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said Q³ is mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl;
or Z is Z³; and Z³ forms a 5-7 membered heteroaryl or heteroalkyl ring with R³, optionally said heteroaryl or heteroalkyl each independently having one or two additional heteroatoms selected independently from nitrogen, sulfur and oxygen having the formula X5

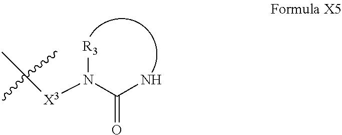

with the proviso that the compound of Formula (I) is not N—({1-[(trans-4-tert-butylcyclohexyl)({[4-(trifluoromethoxy)phenyl]amino}carbonyl]amino)-2,3-dihydro-1H-inden-5-yl}carbonyl)-B-alanine.

Also provided herein are pharmaceutical compositions comprising a compound of Formula (I) or a pharmaceutically acceptable salt thereof and a pharmaceutically acceptable excipient.

Also provided herein is a pharmaceutical combination composition comprising: a therapeutically effective amount of a composition comprising:
a first compound, said first compound being a compound of Formula I or a pharmaceutically acceptable salt of said compound;
a second compound, said second compound being a GLP-1 inhibitor or a GCGR agonist; and a pharmaceutically acceptable excipient.

Also provided herein is a pharmaceutical combination composition comprising: a therapeutically effective amount of a composition comprising:
a first compound, said first compound being a compound of Formula I or a pharmaceutically acceptable salt of said compound;
a second compound, said second compound being a GLP-1 inhibitor; and
a third compound, said third compound being a GCGR agonist; and
a pharmaceutically acceptable excipient.

Also provided herein is a kit comprising:
a. a first compound, said first compound being a compound of Formula I, a prodrug thereof, or a pharmaceutically acceptable salt of said compound or of said prodrug and a pharmaceutically acceptable excipient in a first unit dosage form;
b. a second compound, said second compound being a GLP-1 inhibitor or a GCGR agonist and a pharmaceutically acceptable excipient in a second unit dosage form; and
c. means for containing said first and second dosage forms
wherein the amounts of first and second compounds result in a therapeutic effect.

Also provided herein is a kit wherein said second compound is a GLP-1 inhibitor.

Further provided herein are methods of modulating GIPR activity; and/or methods of treating a GIPR-mediated disease or disorder in a subject in need thereof, wherein each of these methods independently comprises administering an effective amount of a compound of Formula (I) or a pharmaceutically acceptable salt thereof, or administering an effective amount of a pharmaceutical composition comprising a compound of Formula (I) or a pharmaceutically acceptable salt thereof and a pharmaceutically acceptable excipient.

Also provided is a method of treating a condition chosen from Type 2 diabetes, elevated glucose levels, elevated insulin levels, dyslipidemia, metabolic syndrome (Syndrome X or insulin resistance syndrome), glucosuria, metabolic acidosis, Type 1 diabetes, obesity, conditions exacerbated by obesity, pancreatitis, cirrhosis and Turner's & Cushing's syndrome comprising administering to the subject an effective amount of the compound of Formula (I) and/or a pharmaceutically acceptable salt thereof, or a pharmaceutical composition as described herein.

Also provided herein is a method of treating a disease or condition chosen from Type 2 diabetes, elevated glucose levels, elevated insulin levels, dyslipidemia, metabolic syndrome (Syndrome X or insulin resistance syndrome), glucosuria, metabolic acidosis, Type 1 diabetes and obesity, conditions exacerbated by obesity, pancreatitis, cirrhosis and Turner's & Cushing's syndrome comprising administering to the subject an effective amount of a compound of Formula I or a pharmaceutical composition thereof; and a second compound, said second compound being a GLP-1 inhibitor or a GCGR agonist.

Also provided herein are compounds of Formula (I) or a pharmaceutically acceptable salt thereof for use as a medicament. In some embodiments the compound is for use in treating disease or disorder mediated by GIPR.

Also provided herein is the use of a compound of Formula (I) or a pharmaceutically acceptable salt thereof in the manufacture of a medicament for treating a GIPR-mediated disease or disorder.

### DETAILED DESCRIPTION

Unless otherwise stated, the following terms used in the specification and claims are defined for the purposes of this disclosure and have the following meanings.

“Alkyl” means a saturated, straight or branched hydrocarbon moiety having the specified number of carbon atoms. The term “(C₁-C₆)alkyl” refers to an alkyl moiety containing from 1 to 6 carbon atoms. Exemplary alkyls include, but are not limited to, methyl, ethyl, n-propyl, isopropyl, n-butyl, isobutyl, s-butyl, t-butyl, pentyl, and hexyl. and the like.

“Alkenyl” means a saturated, straight or branched hydrocarbon moiety having the specified number of carbon atoms and at least one carbon-carbon double bond. The term “(C₁-C₆)alkenyl” refers to an alkenyl moiety containing from 1 to 6 carbon atoms. Exemplary alkenyls include, but are not limited to, ethenyl, n-propenyl, isopropenyl, n-butenyl, isobutenyl, s-butenyl, t-butenyl, pentenyl, and hexenyl. and the like.

“Alkoxy” means a —OR radical where R is alkyl as defined above, e.g., methoxy, ethoxy, propoxy, or 2-propoxy, n-, iso-, or tert-butoxy, and the like. The term “(C₁-C₄)alkoxy” refers to a straight- or branched-chain hydrocarbon radical having at least 1 and up to 4 carbon atoms attached through an oxygen linking atom.

When the term “alkyl” is used in combination with other substituent groups, such as “halo(C₁-C₄)alkyl”, “aryl(C₁-C₄)alkyl”, or “(C₁-C₄)alkoxy(C₁-C₄)alkyl”, the term “alkyl” is intended to encompass a divalent straight or branched-chain hydrocarbon radical, wherein the point of attachment is through the alkyl moiety. The term “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical. Examples of “halo(C₁-C₄)alkyl” groups useful in the present invention include, but are not limited to, CF₃ (trifluoromethyl), CCl₃ (trichloromethyl), 1,1-difluoroethyl, 2-fluoro-2-methylpropyl, 2,2-difluoropropyl, 2,2,2-trifluoroethyl, and hexafluoroisopropyl. Examples of “aryl(C₁-C₄)alkyl” or “phenyl(C₁-C₄)alkyl” groups useful in the present invention include, but are not limited to, benzyl and phenethyl. Examples of “(C₁-C₄)alkoxy(C₁-C₄)alkyl” groups useful in the present invention include, but are not limited to, methoxymethyl, methoxyethyl, methoxyisopropyl, ethoxymethyl, ethoxyethyl, ethoxyisopropyl, isopropoxymethyl, isopropoxyethyl, isopropoxyisopropyl, t-butoxymethyl, t-butoxy ethyl, and t-butoxyisopropyl. Aryl groups may include heteroatoms only if specifically stated to include heteroatoms such as in the description of X¹.

As used herein, the term “cycloalkyl” refers to a non-aromatic, saturated or unsaturated, cyclic hydrocarbon ring containing the specified number of carbon atoms. The term (C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

As used herein, “heterocycloalkyl” represents a group or moiety comprising a nonaromatic, monovalent monocyclic radical, which is saturated or partially unsaturated and includes one, two or three heteroatoms selected independently from oxygen, sulfur, and nitrogen. For example, the term “4- to 6-membered heterocycloalkyl” refers to a heterocycloalkyl group containing 4, 5, or 6 ring atoms, which includes one or two heteroatoms selected independently from oxygen, sulfur, and nitrogen. Illustrative examples of 4- to 6-membered heterocycloalkyl groups useful in the present invention include, but are not limited to azetidinyl, oxetanyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiol any 1, 1,3-oxathianyl, 1,3-dithianyl, 1,4-oxathiolanyl, 1,4-oxathianyl, and 1,4-dithianyl.

As used herein, “amino” refers to primary, secondary and tertiary nitrogen containing groups. Mono-N, or di-N,N (C₁-Cₓ)alkylamino refers to a single (C₁-C₄)alkyl substituent bonded to an amino moiety (mono-N) or two (C₁-Cₓ)alkyl substituents bonded to an amino (di-N,N), wherein the two (C₁-Cₓ)alkyl substituents may be the same or different and x represents the maximum number of carbon atoms in the alkyl substituent.

“Halo” means fluoro, chloro, bromo, or iodo; in one embodiment the halo is fluoro or chloro.

As used herein (unless otherwise specified), “aryl” refers to monovalent monocyclic aromatic ring having 5-8 carbon atoms that complies with Huckel's Rule. As used herein, “arylene” refers to a bivalent aryl group. When specified, fused bicyclic, or fused tricyclic groups having 8 to 16 carbon atoms and having at least one aromatic ring that complies with Huckel's Rule are provided. Examples of “aryl” groups are phenyl, naphthyl, indenyl, dihydroindenyl, anthracenyl, phenanthrenyl, and the like. Bicyclic and tricyclic aryl rings may contain an aryl ring that is fused to a cycloalkyl ring moiety, containing 5 to 16 ring atoms.

“Heteroaryl” as used herein (unless otherwise specified) refers to a group or moiety comprising an aromatic monovalent monocyclic ring containing 5 to 8 ring atoms. When specified a bicyclic ring, or a tricyclic ring containing 8 to 16 ring atoms, including 1 to 6 heteroatoms independently selected from nitrogen, oxygen and sulfur is provided. The bicyclic and tricyclic heterocyclic-aryl compounds containing an aryl ring moiety fused to a heterocycloalkyl ring moiety, containing 5 to 16 ring atoms, including 1 to 4 heteroatoms independently selected from nitrogen, oxygen and sulfur are also provided. Illustrative examples of heteroaryl groups useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 5-membered “heteroaryl” groups include furanyl, thienyl, pyrrolyl, imidazolyl, pyrazolyl, triazolyl, tetrazolyl, thiazolyl, oxazolyl, isoxazolyl, oxadiazolyl, thiadiazolyl, and isothiazolyl. Examples of 6-membered “heteroaryl” groups include oxo-pyridyl, pyridinyl, pyridazinyl, pyrazinyl, and pyrimidinyl. Examples of 6,6-fused “heteroaryl” groups include quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 6,5-fused “heteroaryl” groups include benzofuranyl, benzothienyl, benzimidazolyl, benzthiazolyl, indolizinyl, indolyl, isoindolyl, and indazolyl. Examples of 10- to 16-membered tricyclic heteroaryl group include [1,3]dioxolo[4′,5′:4,5]benzo[1,2-d]thiazole groups, and the like.

As used herein, (unless otherwise specified) heteroaryl represents a moiety comprising an aromatic monovalent monocyclic ring, including at least one carbon atom and 1 to 4 heteroatoms independently selected from nitrogen, oxygen, and sulfur. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Illustrative examples of 5- or 6-membered heteroaryl groups useful in the present invention include, but are not limited to furanyl, thienyl, pyrrolyl, imidazolyl, pyrazolyl, triazolyl, tetrazolyl, oxazolyl, thiazolyl, isoxazolyl, isothiazolyl, oxadiazolyl, thiadiazolyl, pyridinyl, pyridazinyl, pyrazinyl, pyrimidinyl, and triazinyl. As used herein, “heteroarylene” refers to a bivalent heteroaryl group. As used herein, “hetero(C₅-C₆)aryl” refers to a heteroaryl group as defined above wherein 5-6 ring members are carbon atoms.

“Oxo” means an=(O) group where the oxygen is bound to any atom and “carbonyl” means a >C(O) or >C═O or C═O group.

“Mammal” as used herein means domesticated animals (such as dogs, cats, and horses), and humans. In one embodiment, mammal is a human, male or female.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

The term “salt” or “pharmaceutically acceptable salt” refers to salts derived from a variety of organic and inorganic counter ions well known in the art. In some embodiments, due to the acidic nature of the compounds of this invention, the pharmaceutically acceptable salts form with base addition salts.

Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

In some embodiments, the pharmaceutically acceptable base addition salt is chosen from ammonium, potassium, sodium, calcium, and magnesium salts.

It is understood that the pharmaceutically acceptable salts are non-toxic. For a review on suitable salts, see Handbook of Pharmaceutical Salts. Properties, Selection, and Use by Stahl and Wermuth (Wiley-VCH, 2002). Additional information on suitable pharmaceutically acceptable salts can be found in Remington The Science and Practice of Pharmacy, 23rd ed., Elsevier Science, 2020, which is incorporated herein by reference.

“Optional” or “optionally” means that the subsequently described event or circumstance may but need not occur, and that the description includes instances where the event or circumstance occurs and instances in which it does not. For example, “heterocycloalkyl group optionally substituted with an alkyl group” means that the alkyl may but need not be present, and the description includes situations where the heterocycloalkyl group is substituted with an alkyl group and situations where the heterocycloalkyl group is not substituted with alkyl.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” (includes pharmaceutically acceptable carriers, stabilizers and the like) as described herein. used herein means a

“Treating” or “treatment” of a disease includes:
(1) preventing the disease, e.g., causing the clinical symptoms of the disease not to develop in a mammal that may be exposed to or predisposed to the disease but does not yet experience or display symptoms of the disease;
(2) inhibiting the disease, e.g., arresting or reducing the development or extent of the disease or its clinical symptoms; or
(3) relieving the disease, e.g., causing regression of the disease or its clinical symptoms.

Thus, prevent, preventing, and the like can refer to the prevention of the disease or condition in the patient. For example, if an individual at risk of contracting a condition/disease is treated with the methods of the present disclosure and does not later contract the condition/disease, then the disease has been prevented, at least over a period of time, in that individual.

A “therapeutically effective amount” means the amount of a compound of Formula (I) (or any of the embodiments thereof described herein), or combination thereof, that when administered to a mammal for treating a disease, is sufficient to treat the disease. The “therapeutically effective amount” will vary depending on the compound, the disease and its severity and the age, weight, etc., of the mammal to be treated.

The compounds described herein may in some cases exist as diastereomers, enantiomers, or other stereoisomeric forms. All chiral, diastereomeric, racemic forms, as individual forms and mixtures thereof, are within the scope of this disclosure, unless the specific stereochemistry or isomeric form is specifically indicated. Compounds of the present disclosure containing an asymmetrically substituted atom may be isolated in optically active, optically enriched, optically pure, or racemic forms. It is well known in the art how to prepare optically active forms, such as by resolution of materials. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis. Those skilled in the art are familiar with methods for determining absolute stereochemistry e.g., X-Ray powder diffraction. Stereoisomers may also be obtained by stereoselective synthesis into their compounding pure enantiomers by forming a diastereomeric salt with an optically pure chiral base or acid (e.g., 1-phenyl-ethylamine or tartaric acid) and separating the diastereomers by fractional crystallization followed by neutralization to break the salt, thus providing the corresponding pure enantiomers.

Certain compounds of Formula (I) (or any of the embodiments thereof described herein) and/or a pharmaceutically acceptable salt thereof may exist as tautomers and/or geometric isomers. All possible tautomers and cis and trans isomers, as individual forms and mixtures thereof, are within the scope of this disclosure.

Additionally, as used herein the term alkyl includes all the possible isomeric forms of said alkyl group albeit only a few examples are set forth. Furthermore, when the cyclic groups such as heteroaryl, heterocyclyl are substituted, they include all the positional isomers.

The compounds described herein include hydrates and solvates of the compounds or pharmaceutically acceptable salts thereof. The term solvate is used herein to describe a molecular complex comprising the compound of the invention and one or more pharmaceutically acceptable solvent molecules, for example, ethanol. Such solvent molecules are those commonly used in the pharmaceutical art, which are known to be innocuous to the recipient, e.g., water, ethanol, and the like. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The term hydrate is employed when the solvent is water. Pharmaceutically acceptable solvates include hydrates and other solvates wherein the solvent of crystallization may be isotopically substituted, e.g., D₂O. d-acetone, d-DMSO. The solvates and/or hydrates preferably exist in crystalline form. A classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

The present disclosure also includes prodrugs of compounds of Formula (I) (or any of the embodiments thereof described herein) and/or a pharmaceutically acceptable salt thereof. The term prodrug is intended to represent covalently bonded carriers, which are capable of releasing the active ingredient of Formula (I) (or any of the embodiments thereof described herein) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups, however, regenerate original functional groups in vivo or by routine manipulation. Prodrugs of compounds of Formula (I) (or any of the embodiments thereof described herein) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) (or any of the embodiments thereof described herein) and/or a pharmaceutically acceptable salt thereof are also within the scope of this disclosure.

Also included within the scope of the invention are multi-component complexes (other than salts and solvates) wherein the drug and at least one other component are present in stoichiometric or non-stoichiometric amounts. The compounds of the invention may also exist as complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975).

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins.

The present disclosure also includes polymorphic forms (amorphous as well as crystalline). The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Certain compounds of the present invention or combination agents may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

The compounds disclosed herein, in some embodiments, are used in different enriched isotopic forms. Thus, the compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention. For example, compounds having the present structures except for the replacement of a hydrogen by a deuterium or tritium, or the replacement of a carbon by ¹³C- or ¹⁴C-enriched carbon are within the scope of the present disclosure. In certain embodiments, the compounds disclosed herein have some or all of the ¹H atoms replaced with ²H atoms. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs. Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32. Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Also included within the scope of the invention are metabolites of compounds of Formula I, that is, compounds formed in vivo upon administration of the drug. Some examples of metabolites in accordance with the invention include
(i) where the compound of Formula I contains a methyl group, an hydroxymethyl derivative thereof (—CH₃→—CH₂OH):
(ii) where the compound of Formula I contains an alkoxy group, an hydroxy derivative thereof (—OR→—OH);
(iii) where the compound of Formula I contains a tertiary amino group, a secondary amino derivative thereof (—NRR→—NHR or —NHR);
(iv) where the compound of Formula I contains a secondary amino group, a primary derivative thereof (—NHR→—NH₂);
(v) where the compound of Formula I contains a phenyl moiety, a phenol derivative thereof (-Ph→-PhOH); and
(vi) where the compound of Formula I contains an amide group, a carboxylic acid derivative thereof (—CONH₂→COOH).

The present invention may be understood by reference to the following detailed description of exemplary aspects/embodiments of the invention and the examples included herein.

In some embodiments, provided herein is a compound of Formula (I) or a pharmaceutically acceptable salt thereof:

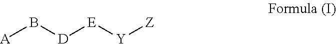

wherein
A is —C(O)OR² or —S(O)₂OR²;
R² is H or (C₁-C₆)alkyl;
B is (C₁-C₄)alkylene;
D is NH, CH₂, or is absent;
E is carbonyl, sulfonyl, NH, CH₂ or is absent;
Y is

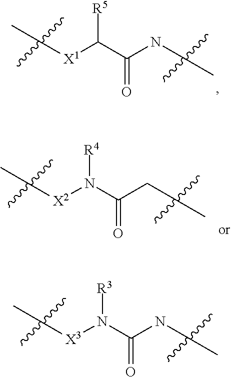

wherein

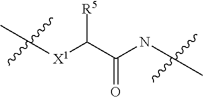

when Y is
X¹ is a bicyclic ring comprising a 5-6 membered aryl ring, optionally having 0 to 3 heteroatoms selected independently from nitrogen, oxygen and sulfur, fused to a (C₅-C₇)cycloalkyl ring optionally containing 0 to 3 heteroatoms independently selected from nitrogen, oxygen and sulfur;
X¹ is optionally mono-, di- or tri-substituted independently with (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, difluoromethoxy or trifluoromethoxy;
R⁵ is (C₅-C₆)alkyl, phenyl, hetero(C₅-C₆)aryl, (C₃-C₇)cycloalkyl, (C₃-C₇)cycloalkenyl or hetero(C₃-C₇)cycloalkyl; said hetero(C₅-C₆)aryl and hetero(C₃-C₇)cycloalkyl each independently having 1 to 3 heteroatoms selected independently from nitrogen, oxygen and sulfur;
wherein said R⁵ is optionally mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, pentafluoropropanyl, mono-N— or di-N,N—(C₁-C₆)alkylaminocarbonyl, mono-N— or di-N,N—(C₁-C₆)alkylaminocarbonyloxy, (C₁-C₆)alkylcarbonyloxy, (C₁-C₆)alkylcarbonyl or (C₁-C₆)alkoxycarbonyl;
or R⁵ forms a 5-7 membered heteroaryl or heteroalkyl ring with nitrogen, optionally said heteroaryl or heteroalkyl ring each independently having one or two additional heteroatoms selected independently from nitrogen, sulfur and oxygen having the Formula X, wherein said 5-7 membered ring formed with nitrogen is optionally substituted with (C₁-C₄)alkyl, or is mono- or di-substituted independently with T¹;

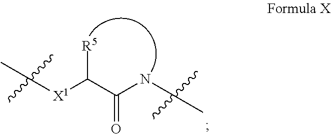

wherein T¹ is phenoxy, hetero(C₅-C₆)aryloxy, (C₃-C₇)cycloalkoxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl;
wherein said hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said T¹ is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; and
Z is Z¹; and Z¹ is phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryl or hetero(C₃-C₇)cycloalkyl each independently have 1 to 3 heteroatoms selected independently from oxygen, nitrogen and sulfur;
wherein said Z¹ is mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, (C₃-C₆)cycloalkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; or wherein said Z¹ is mono- or di-substituted independently with Q¹;
wherein Q¹ is phenoxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl;
wherein hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said Q¹ is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl;
or Z is Z¹ and Z¹ forms a 5-7 membered heteroaryl or heteroalkyl ring with R⁵, optionally said heteroaryl or heteroalkyl each independently having one or two additional heteroatoms selected independently from nitrogen, sulfur and oxygen having the Formula XI:

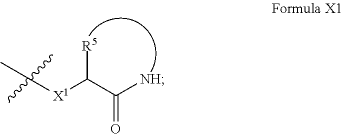

or
when Y is

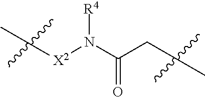

X² is a bicyclic ring comprising a 5-6 membered aryl ring optionally having 0 to 3 heteroatoms selected independently from nitrogen, oxygen and sulfur fused to a (C₅-C₇)cycloalkyl ring optionally containing 0 to 3 heteroatoms independently selected from nitrogen, oxygen and sulfur;
X² is optionally mono-, di- or tri-substituted with (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, difluoromethoxy or trifluoromethoxy;
R⁴ is (C₅-C₆)alkyl, phenyl, hetero(C₅-C₆)aryl, (C₃-C₇)cycloalkyl, (C₃-C₇)cycloalkenyl or hetero(C₃-C₇)cycloalkyl; said hetero(C₅-C₆)aryl and hetero(C₃-C₇)cycloalkyl each independently having 1 to 3 heteroatoms selected independently from nitrogen, oxygen and sulfur;
wherein said R⁴ is optionally mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, pentafluoropropanyl, mono-N— or di-N,N—(C₁-C₆)alkylaminocarbonyl, mono-N— or di-N,N—(C₁-C₆)alkylaminocarbonyloxy, (C₁-C₆)alkylcarbonyloxy, (C₁-C₆)alkylcarbonyl or (C₁-C₆)alkoxycarbonyl;
or R⁴ forms a 5-7 membered heteroaryl or heteroalkyl ring with nitrogen, said heteroaryl or heteroalkyl ring each optionally having one or two additional heteroatoms selected independently from nitrogen, sulfur and oxygen having the Formula X2; wherein said ring formed with nitrogen is optionally substituted with (C₁-C₄)alkyl; or wherein said R⁴ is mono- or di-substituted independently with T²;

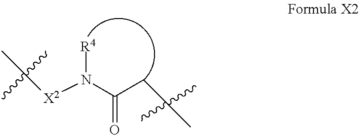

wherein T² is phenoxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said T² is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; and
Z is Z²; and Z² is phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryl or hetero(C₃-C₇)cycloalkyl each independently have 1 to 3 heteroatoms selected independently from oxygen, nitrogen and sulfur;
wherein said Z² is mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkoxy(C₁-C₆)alkyl, difluoromethyl, trifluoromethyl, trifluoroethyl, trifluoromethylthio, pentafluoroethanyl, or pentafluoropropanyl; or Z² is mono- or di-substituted independently with Q²
wherein Q² is phenoxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said Q² is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl;
or Z is Z²; and Z² forms a 5-7 membered heteroaryl or heteroalkyl ring with R⁴, optionally said heteroaryl or heteroalkyl each independently having one or two additional heteroatoms selected independently from nitrogen, sulfur and oxygen having the formula X3

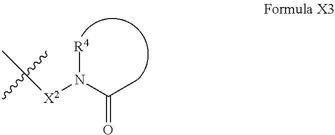

or
when Y is

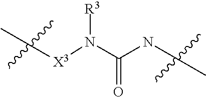

X³ is a bicyclic ring comprising a 5-6 membered aryl ring optionally having 0-3 heteroatoms selected independently from nitrogen, oxygen and sulfur fused to a (C₅-C₇)cycloalkyl ring optionally containing 0 to 3 heteroatoms independently selected from nitrogen, oxygen and sulfur;
X³ is optionally mono-, di- or tri-substituted with (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, difluoromethoxy or trifluoromethoxy;
R³ is (C₅-C₆)alkyl, phenyl, hetero(C₅-C₆)aryl, (C₃-C₇)cycloalkyl, (C₃-C₇)cycloalkenyl or hetero(C₃-C₇)cycloalkyl; said hetero(C₅-C₆)aryl and hetero(C₃-C₇)cycloalkyl each independently having 1 to 3 heteroatoms selected independently from nitrogen, oxygen and sulfur;
wherein said R³ is optionally mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, pentafluoropropanyl, mono-N— or di-N,N—(C₁-C₆)alkylaminocarbonyl, mono-N— or di-N,N—(C₁-C₆)alkylaminocarbonyloxy, (C₁-C₆)alkylcarbonyloxy, (C₁-C₆)alkylcarbonyl or (C₁-C₆)alkoxycarbonyl;
or R³ forms a 5-7 membered heteroaryl or heteroalkyl ring with nitrogens, said heteroaryl or heteroalkyl ring each optionally having one or two additional heteroatoms selected independently from nitrogen, sulfur and oxygen having the formula X4 wherein said ring formed with nitrogens is optionally substituted with (C₁-C₄)alkyl; or wherein said R³ is mono- or di-substituted independently with T³;

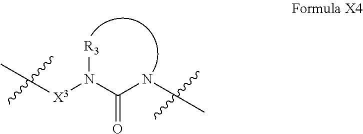

wherein T³ is phenyloxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said T³ is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; and
Z is Z³; and Z³ is phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryl or hetero(C₃-C₇)cycloalkyl each independently have 1 to 3 heteroatoms selected independently from oxygen, nitrogen and sulfur;
wherein said Z³ is mono-, di- or tri-substituted independently with cyano, cyanomethyl, nitro, amino, thio, mono-N— or di-N,N—(C₁-C₆)alkylamino, mono-N— or di-N,N—(C₁-C₆)alkylamino(C₁-C₆)alkyl, (C₃-C₆)cycloalkyl, (C₁-C₆)alkoxy(C₁-C₆)alkyl, difluoromethyl, trifluoroethyl, trifluoromethylthio, pentafluoroethanyl, or pentafluoropropanyl; or Z³ is mono- or di-substituted independently with Q³;
wherein Q³ is phenyloxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said Q³ is mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl;
or Z is Z³; and Z³ forms a 5-7 membered heteroaryl or heteroalkyl ring with R³, optionally said heteroaryl or heteroalkyl each independently having one or two additional heteroatoms selected independently from nitrogen, sulfur and oxygen having the formula X5

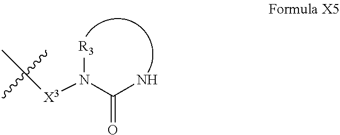

with the proviso that the compound of Formula (I) is not N—({1-[(trans-4-tert-butylcyclohexyl)({[4-(trifluoromethoxy)phenyl]amino}carbonyl]amino)-2,3-dihydro-1H-inden-5-yl}carbonyl)-B-alanine.

In some embodiments of the compound of Formula (I), A is —C(O)OH; B is (C₁-C₄)alkylene; D is NH; E is carbonyl;
Y is

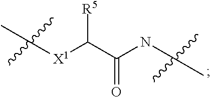

X¹ is 2,3-dihydro-1H-indenyl, indolinyl, tetrahydroquinolinyl or tetrahydronaphthalenyl;
wherein said X¹ is optionally mono-substituted with (C₁-C₆)alkyl or trifluoromethyl;
R⁵ is phenyl or (C₃-C₇)cycloalkyl; said phenyl or (C₃-C₇)cycloalkyl mono-substituted with (C₁-C₆)alkyl, phenyl or (C₃-C₇)cycloalkyl; and Z¹ is phenyl mono-, di- or tri-substituted independently with (C₁-C₆)alkyl, (C₁-C₆)alkoxy, trifluoromethyl or trifluoromethylthio; or said Z¹ is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio. In some embodiments, R⁵ is phenyl or (C₅-C₈)cycloalkyl; said phenyl or C₅-C₈)cycloalkyl mono-substituted with (C₁-C₆)alkyl. In some embodiments, R⁵ is phenyl or (C₃-C₇)cycloalkyl; said phenyl or (C₃-C₇)cycloalkyl mono-substituted with phenyl or (C₃-C₇)cycloalkyl. In some embodiments, Z¹ is phenyl; said phenyl mono-, di- or tri-substituted independently with (C₁-C₆)alkyl, (C₁-C₆)alkoxy, trifluoromethyl or trifluoromethylthio, In some embodiments, Z¹ is phenyl, said phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio. In some embodiments, R⁵ is phenyl or (C₃-C₇)cycloalkyl; said phenyl or (C₃-C₇)cycloalkyl mono-substituted with phenyl or (C₃-C₇)cycloalkyl; and Z¹ is phenyl which is mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromeththio.

In some embodiments of the compound of Formula (I), A is —C(O)OH; B is (C₁-C₄)alkylene; D is NH; E is carbonyl;
Y is

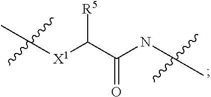

X¹ is 2,3-dihydro-1H-indenyl, indolinyl, tetrahydroquinolinyl or tetrahydronaphthalenyl; X¹ is optionally mono-substituted with (C₁-C₆)alkyl or trifluoromethyl; R⁵ forms a 5-7 membered heteroaryl or heteroalkyl ring with nitrogen, optionally said heteroaryl or heteroalkyl ring each independently having one or two additional heteroatoms selected independently from nitrogen, sulfur and oxygen having the formula X, wherein said ring formed with nitrogen is optionally substituted with (C₁-C₄)alkyl;

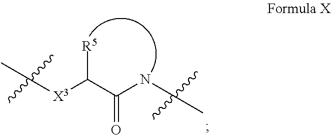

Z¹ is phenyl mono-, di- or tri-substituted independently with (C₁-C₆)alkyl, (C₁-C₆)alkoxy, trifluoromethyl or trifluoromethylthio; or Z¹ is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio. In some embodiments, Z¹ is phenyl; said phenyl mono-, di- or tri-substituted independently with (C₁-C₆)alkyl, (C₁-C₆)alkoxy, trifluoromethyl or trifluoromethylthio, In some embodiments, Z¹ is phenyl which is mono-substituted with (C₃-C₇)cycloalkyl, phenyl, phenoxy or benzyl wherein said (C₃-C₇)cycloalkyl, phenyl, phenoxy, or benzyl are mono-, di- or tri-substituted independently with (C₁-C₆)alkyl, (C₁-C₆)alkoxy, trifluoromethyl or trifluoromethylthio.

In some embodiments of the compound of Formula (I), A is —C(O)OH; B is (C₁-C₄)alkylene; D is NH; E is carbonyl;
Y is

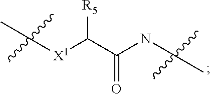

X¹ is 2,3-dihydro-1H-indenyl, indolinyl, tetrahydroquinolinyl or tetrahydronaphthalenyl; X¹ is optionally mono-substituted with (C₁-C₆)alkyl or trifluoromethyl; R⁵ is phenyl or (C₃-C₇)cycloalkyl; said phenyl or (C₃-C₇)cycloalkyl mono-substituted with (C₁-C₆)alkyl, phenyl or (C₃-C₇)cycloalkyl; Z¹ forms a 5-7 membered heteroaryl or heteroalkyl ring with R⁵, optionally said heteroaryl or heteroalkyl each independently having one or two additional heteroatoms selected independently from nitrogen, sulfur and oxygen having the formula XI;

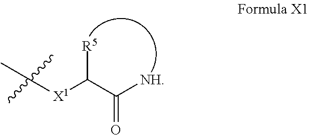

In some embodiments of the compound of Formula (I), A is —C(O)OH; B is (C₁-C₄)alkylene; D is NH; E is carbonyl;
Y is

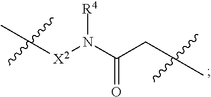

X² is 2,3-dihydro-1H-indenyl, indolinyl, tetrahydroquinolinyl or tetrahydronaphthalenyl;
wherein said X² is optionally mono-substituted with (C₁-C₆)alkyl or trifluoromethyl;
R⁴ is phenyl or (C₃-C₇)cycloalkyl; said phenyl or (C₃-C₇)cycloalkyl mono-substituted with (C₁-C₆)alkyl, phenyl or (C₃-C₇)cycloalkyl; Z² is phenyl mono-, di- or tri-substituted independently with (C₁-C₆)alkoxy(C₁-C₆)alkyl, difluoromethyl, trifluoromethyl, trifluoroethyl or trifluoromethylthio; or Z² is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, (C₁-C₆)alkoxy(C₁-C₆)alkyl, difluoromethyl, trifluoroethyl, trifluoromethylthio, fluoromethyloxy, difluoromethyloxy or trifluoromethyloxy, In some embodiments, R⁴ is phenyl or (C₅-C₈)cycloalkyl; said phenyl or C₅-C₈)cycloalkyl mono-substituted with (C₁-C₆)alkyl. In some embodiments, R⁴ is phenyl or (C₃-C₇)cycloalkyl; said phenyl or (C₃-C₇)cycloalkyl mono-substituted with phenyl or (C₃-C₇)cycloalkyl. In some embodiments, Z² is phenyl mono-, di- or tri-substituted independently with (C₁-C₆)alkoxy(C₁-C₆)alkyl, difluoromethyl, trifluoromethyl, trifluoroethyl or trifluoromethylthio. In some embodiments, Z² is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio. In some embodiments, R⁴ is phenyl or (C₃-C₇)cycloalkyl; said phenyl or (C₃-C₇)cycloalkyl mono-substituted with phenyl or (C₃-C₇)cycloalkyl; and Z² is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio.

In some embodiments of the compound of Formula (I), A is —C(O)OH; B is (C₁-C₄)alkylene;
D is NH; E is carbonyl;
Y is

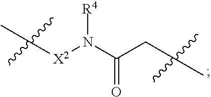

X² is 2,3-dihydro-1H-indenyl, indolinyl, tetrahydroquinolinyl or tetrahydronaphthalenyl; X² is optionally mono-substituted with (C₁-C₆)alkyl or trifluoromethyl; R⁴ forms a 5-7 membered aryl, heteroaryl or heteroalkyl ring with nitrogen, optionally said heteroaryl or heteroalkyl ring each independently having one or two additional heteroatoms selected independently from nitrogen, sulfur and oxygen having the formula X2, wherein said ring formed with nitrogen is optionally substituted with (C₁-C₄)alkyl;

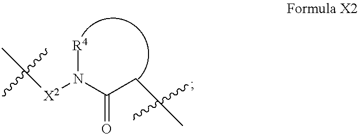

Z² is phenyl mono-, di- or tri-substituted independently with (C₁-C₆)alkoxy(C₁-C₆)alkyl, difluoromethyl, trifluoromethyl, trifluoroethyl or trifluoromethylthio; or Z² is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio. In some embodiments, Z² is phenyl mono-, di- or tri-substituted independently with (C₁-C₆)alkoxy(C₁-C₆)alkyl, difluoromethyl, trifluoromethyl, trifluoroethyl or trifluoromethylthio. In some embodiments, Z² is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio.

In some embodiments of the compound of Formula (I), A is —C(O)OH; B is (C₁-C₄)alkylene; D is NH; E is carbonyl;
Y is

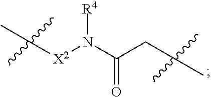

X² is 2,3-dihydro-1H-indenyl, indolinyl, tetrahydroquinolinyl or tetrahydronaphthalenyl; X² is optionally mono-substituted with (C₁-C₆)alkyl or trifluoromethyl; R⁴ is phenyl or (C₃-C₇)cycloalkyl; said phenyl or (C₃-C₇)cycloalkyl mono-substituted with (C₁-C₆)alkyl, phenyl or (C₃-C₇)cycloalkyl; and Z² forms a 5-7 membered heteroaryl or heteroalkyl ring with R⁴, optionally said heteroaryl or heteroalkyl each independently having one or two additional heteroatoms selected independently from nitrogen, sulfur and oxygen having the formula X3

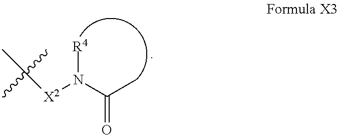

In some embodiments of the compound of Formula (I), A is —C(O)OH; B is (C₁-C₄)alkylene; D is NH; E is carbonyl;
Y is

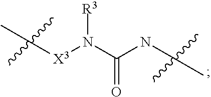

X³ is 2,3-dihydro-1H-indenyl, indolinyl, tetrahydroquinolinyl or tetrahydronaphthalenyl;
wherein said X² is optionally mono-substituted with (C₁-C₆)alkyl or trifluoromethyl; R³ is phenyl or (C₃-C₇)cycloalkyl; said phenyl or (C₃-C₇)cycloalkyl mono-substituted with (C₁-C₆)alkyl, phenyl or (C₃-C₇)cycloalkyl; and Z³ is phenyl mono-, di- or tri-substituted independently with difluoromethyl, trifluoroethyl or trifluoromethylthio; or Z³ is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio. In some embodiments, R³ is phenyl or (C₅-C₈)cycloalkyl; said phenyl or C₅-C₈)cycloalkyl mono-substituted with (C₁-C₆)alkyl. In some embodiments, wherein R³ is phenyl or (C₃-C₇)cycloalkyl; said phenyl or (C₃-C₇)cycloalkyl mono-substituted with phenyl or (C₃-C₇)cycloalkyl. In some embodiments, Z³ is phenyl mono-, di- or tri-substituted independently with difluoromethyl, trifluoroethyl or trifluoromethylthio. In some embodiments, Z³ is phenyl, said phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio. In some embodiments, R³ is phenyl or (C₃-C₇)cycloalkyl; said phenyl or (C₃-C₇)cycloalkyl mono-substituted with phenyl or (C₃-C₇)cycloalkyl; and Z³ is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio.

In some embodiments of the compound of Formula (I), A is —C(O)OH; B is (C₁-C₄)alkylene; D is NH; E is carbonyl;
Y is

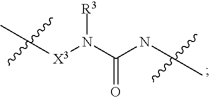

X³ is 2,3-dihydro-1H-indenyl, indolinyl, tetrahydroquinolinyl, or tetrahydronaphthalenyl; X³ is optionally mono-substituted with (C₁-C₆)alkyl or trifluoromethyl; R³ forms a 5-7 membered heteroaryl or heteroalkyl ring with nitrogen, optionally said heteroaryl or heteroalkyl ring each independently having one or two additional heteroatoms selected independently from nitrogen, sulfur and oxygen having the formula X4, wherein said ring formed with nitrogen is optionally substituted with (C₁-C₄)alkyl;

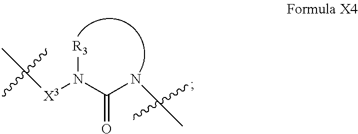

Z³ is phenyl mono-, di- or tri-substituted independently with difluoromethyl, trifluoroethyl or trifluoromethylthio; or Z³ is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio. In some embodiments, Z³ is phenyl mono-, di- or tri-substituted independently with difluoromethyl, trifluoroethyl or trifluoromethylthio. In some embodiments, Z³ is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio.

In some embodiments of the compound of Formula (I), A is —C(O)OH; B is (C₁-C₄)alkylene; D is NH; E is carbonyl;
Y is

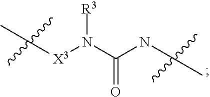

X³ is 2,3-dihydro-1H-indenyl, indolinyl, tetrahydroquinolinyl or tetrahydronaphthalenyl; X³ is optionally mono-substituted with (C₁-C₆)alkyl or trifluoromethyl; R³ is phenyl or (C₃-C₇)cycloalkyl; said phenyl or (C₃-C₇)cycloalkyl mono-substituted with (C₁-C₆)alkyl, phenyl or (C₃-C₇)cycloalkyl; and Z³ forms a 5-7 membered heteroaryl or heteroalkyl ring with R³, optionally said heteroaryl or heteroalkyl each independently having one or two additional heteroatoms selected independently from nitrogen, sulfur and oxygen having the formula X5:

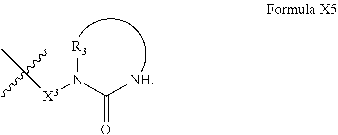

In some embodiments, the compound of Formula (I) is a compound as recited in the Examples and Tables herein, or racemic mixtures thereof, or a pharmaceutically acceptable salt thereof.

The following compounds are provided as examples of this invention.

TABLE 1
List of compounds

| Cmpd | | |
|---|---|---|
| # | Structure | IUPAC Name |
| 1a | 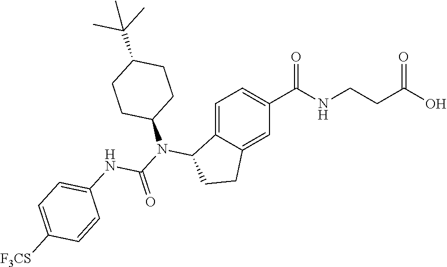 | 3-((S)-1-(1-((1r,4S)-4-(tert- butyl)cyclohexyl)-3-(4- ((trifluoromethyl)thio)phenyl)ureido)-2,3- dihydro-1H-indene-5- carboxamido)propanoic acid |
| 1b | 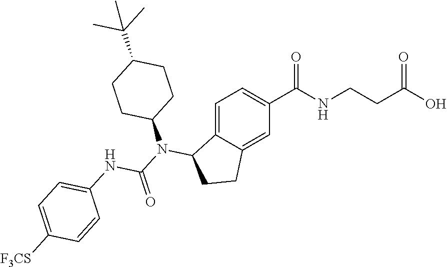 | 3-((R)-1-(1-((1r,4R)-4-(tert- butyl)cyclohexyl)-3-(4- ((trifluoromethyl)thio)phenyl)ureido)-2,3- dihydro-1H-indene-5- carboxamido)propanoic acid |
| 2a | 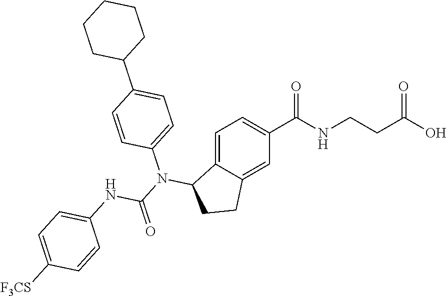 | (R)-3-(1-(1-(4-cyclohexylphenyl)-3-(4- ((trifluoromethyl)thio)phenyl)ureido)-2,3- dihydro-1H-indene-5- carboxamido)propanoic acid |
| 2b | 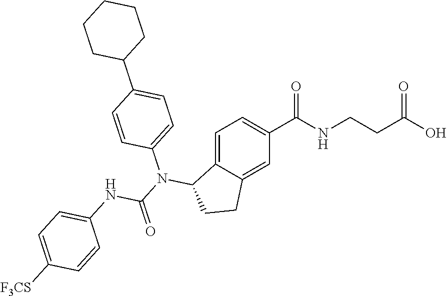 | (S)-3-(1-(1-(4-cyclohexylphenyl)-3-(4- ((trifluoromethyl)thio)phenyl)ureido)-2,3- dihydro-1H-indene-5- carboxamido)propanoic acid |
| 3a | 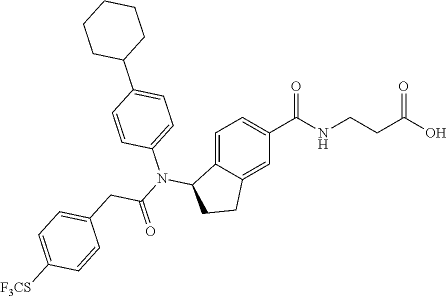 | (R)-3-(1-(N-(4-cyclohexylphenyl)-2-(4- ((trifluoromethyl)thio)phenyl)acetamido)- 2,3-dihydro-1H-indene-5- carboxamido)propanoic acid |
| 3b | 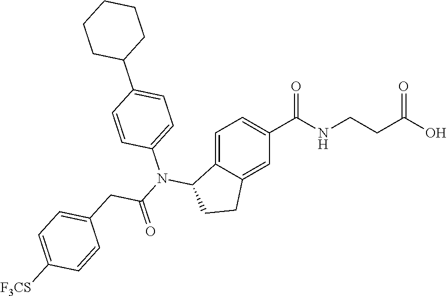 | (S)-3-(1-(N-(4-cyclohexylphenyl)-2-(4- ((trifluoromethyl)thio)phenyl)acetamido)- 2,3-dihydro-1H-indene-5- carboxamido)propanoic acid |
| 4a | 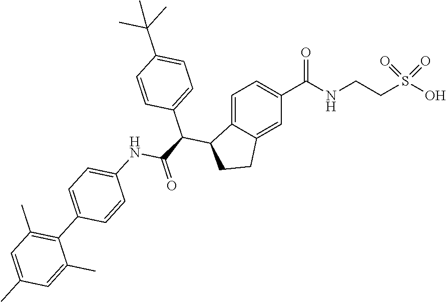 [SMILES: Cc1cc(C)c(-c2ccc(NC(=O)[C@@H](c3ccc(C(C)(C)C)cc3)[C@@H]3CCc4cc(C(=O)NCCS(=O)(=O)O)ccc43)cc2)c(C)c1] | 2-((S)-1-((R)-1-(4-(tert-butyl)phenyl)-2- oxo-2-((2′,4′,6′-trimethyl-[1,1′-biphenyl]- 4-yl)amino)ethyl)-2,3-dihydro-1H- indene-5-carboxamido)ethane-1-sulfonic acid |
| 4b | 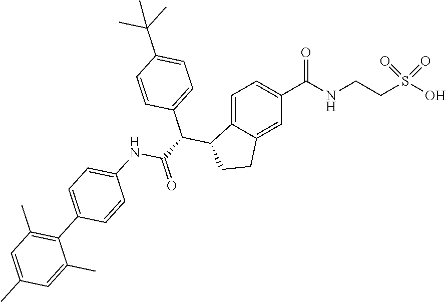 [SMILES: Cc1cc(C)c(-c2ccc(NC(=O)[C@H](c3ccc(C(C)(C)C)cc3)[C@H]3CCc4cc(C(=O)NCCS(=O)(=O)O)ccc43)cc2)c(C)c1] | 2-((R)-1-((S)-1-(4-(tert-butyl)phenyl)-2- oxo-2-((2′,4′,6′-trimethyl-[1,1′-biphenyl]- 4-yl)amino)ethyl)-2,3-dihydro-1H- indene-5-carboxamido)ethane-1-sulfonic acid |
| 4c | 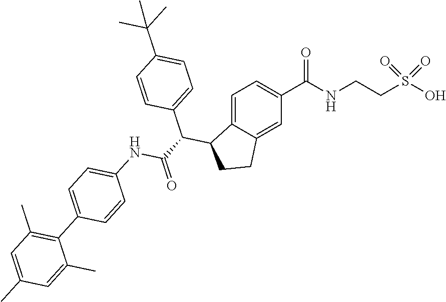 [SMILES: Cc1cc(C)c(-c2ccc(NC(=O)[C@H](c3ccc(C(C)(C)C)cc3)[C@@H]3CCc4cc(C(=O)NCCS(=O)(=O)O)ccc43)cc2)c(C)c1] | 2-((S)-1-((S)-1-(4-(tert-butyl)phenyl)-2- oxo-2-((2′,4′,6′-trimethyl-[1,1′-biphenyl]- 4-yl)amino)ethyl)-2,3-dihydro-1H- indene-5-carboxamido)ethane-1-sulfonic acid |
| 4d | 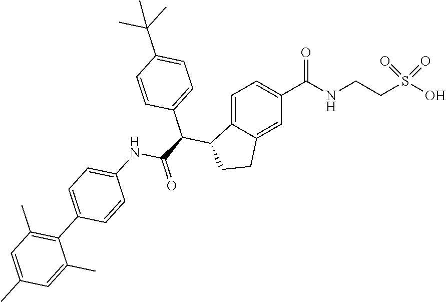 [SMILES: Cc1cc(C)c(-c2ccc(NC(=O)[C@@H](c3ccc(C(C)(C)C)cc3)[C@H]3CCc4cc(C(=O)NCCS(=O)(=O)O)ccc43)cc2)c(C)c1] | 2-((R)-1-((R)-1-(4-(tert-butyl)phenyl)-2- oxo-2-((2′,4′,6′-trimethyl-[1,1′-biphenyl]- 4-yl)amino)ethyl)-2,3-dihydro-1H- indene-5-carboxamido)ethane-1-sulfonic acid |
| 9a | 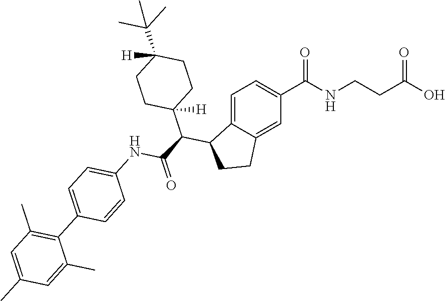 [SMILES: Cc1cc(C)c(-c2ccc(NC(=O)[C@@H]([C@H]3CC[C@H](C(C)(C)C)CC3)[C@@H]3CCc4cc(C(=O)NCCC(=O)O)ccc43)cc2)c(C)c1] | 3-((S)-1-((S)-1-((1r,4S)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)propanoic acid |
| 9b | 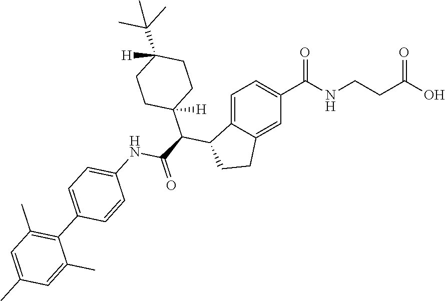 [SMILES: Cc1cc(C)c(-c2ccc(NC(=O)[C@@H]([C@H]3CC[C@H](C(C)(C)C)CC3)[C@H]3CCc4cc(C(=O)NCCC(=O)O)ccc43)cc2)c(C)c1] | 3-((R)-1-((S)-1-((1r,4S)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)propanoic acid |
| 5a | 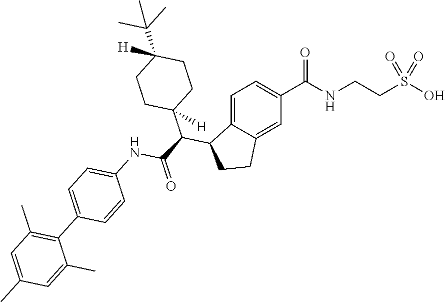 [SMILES: Cc1cc(C)c(-c2ccc(NC(=O)[C@@H]([C@H]3CC[C@H](C(C)(C)C)CC3)[C@@H]3CCc4cc(C(=O)NCCS(=O)(=O)O)ccc43)cc2)c(C)c1] | 2-((S)-1-((S)-1-((1r,4S)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 5b | 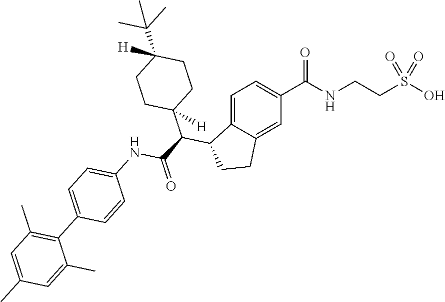 [SMILES: Cc1cc(C)c(-c2ccc(NC(=O)[C@@H]([C@H]3CC[C@H](C(C)(C)C)CC3)[C@H]3CCc4cc(C(=O)NCCS(=O)(=O)O)ccc43)cc2)c(C)c1] | 2-((R)-1-((S)-1-((1r,4S)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 6a | 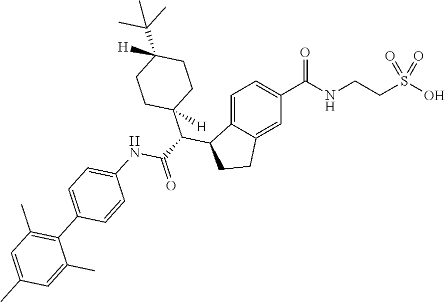 [SMILES: Cc1cc(C)c(-c2ccc(NC(=O)[C@H]([C@H]3CC[C@H](C(C)(C)C)CC3)[C@@H]3CCc4cc(C(=O)NCCS(=O)(=O)O)ccc43)cc2)c(C)c1] | 2-((S)-1-((R)-1-((1r,4R)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 6b | 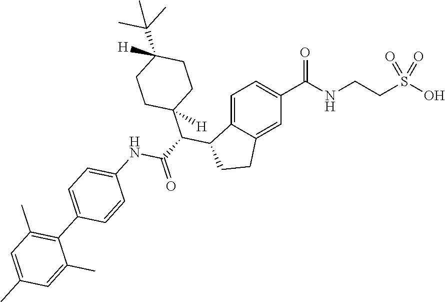 [SMILES: Cc1cc(C)c(-c2ccc(NC(=O)[C@H]([C@H]3CC[C@H](C(C)(C)C)CC3)[C@H]3CCc4cc(C(=O)NCCS(=O)(=O)O)ccc43)cc2)c(C)c1] | 2-((R)-1-((R)-1-((1r,4R)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 7a | 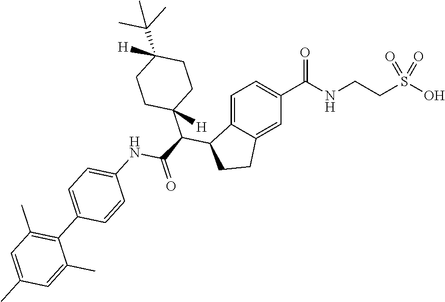 [SMILES: Cc1cc(C)c(-c2ccc(NC(=O)[C@@H]([C@H]3CC[C@@H](C(C)(C)C)CC3)[C@@H]3CCc4cc(C(=O)NCCS(=O)(=O)O)ccc43)cc2)c(C)c1] | 2-((S)-1-((S)-1-((1s,4R)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 7b | 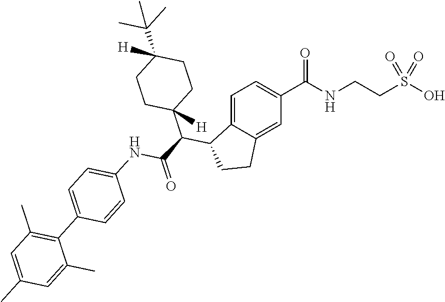 [SMILES: Cc1cc(C)c(-c2ccc(NC(=O)[C@@H]([C@H]3CC[C@@H](C(C)(C)C)CC3)[C@H]3CCc4cc(C(=O)NCCS(=O)(=O)O)ccc43)cc2)c(C)c1] | 2-((R)-1-((S)-1-((1s,4R)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 8a | 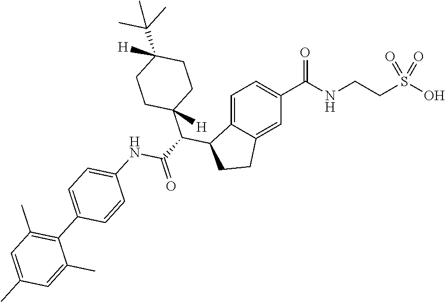 [SMILES: Cc1cc(C)c(-c2ccc(NC(=O)[C@H]([C@H]3CC[C@@H](C(C)(C)C)CC3)[C@@H]3CCc4cc(C(=O)NCCS(=O)(=O)O)ccc43)cc2)c(C)c1] | 2-((S)-1-((R)-1-((1s,4S)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 8b | 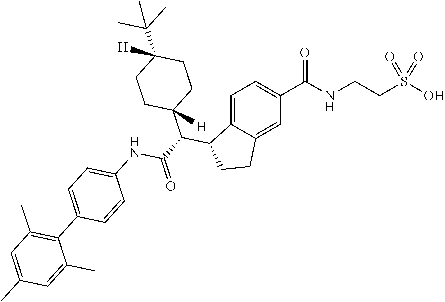 [SMILES: Cc1cc(C)c(-c2ccc(NC(=O)[C@H]([C@H]3CC[C@@H](C(C)(C)C)CC3)[C@H]3CCc4cc(C(=O)NCCS(=O)(=O)O)ccc43)cc2)c(C)c1] | 2-((R)-1-((R)-1-((1s,4S)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 10a | 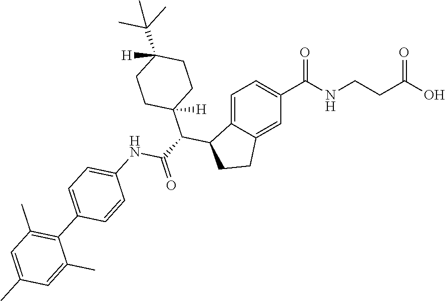 [SMILES: Cc1cc(C)c(-c2ccc(NC(=O)[C@H]([C@H]3CC[C@H](C(C)(C)C)CC3)[C@@H]3CCc4cc(C(=O)NCCC(=O)O)ccc43)cc2)c(C)c1] | 3-((S)-1-((R)-1-((1r,4R)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)propanoic acid |
| 10b | 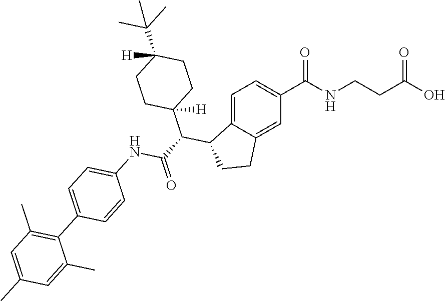 [SMILES: Cc1cc(C)c(-c2ccc(NC(=O)[C@H]([C@H]3CC[C@H](C(C)(C)C)CC3)[C@H]3CCc4cc(C(=O)NCCC(=O)O)ccc43)cc2)c(C)c1] | 3-((R)-1-((R)-1-((1r,4R)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)propanoic acid |
| 11a | 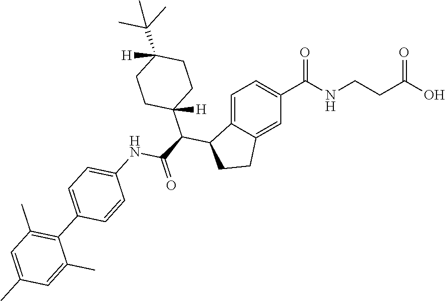 [SMILES: Cc1cc(C)c(-c2ccc(NC(=O)[C@@H]([C@H]3CC[C@@H](C(C)(C)C)CC3)[C@@H]3CCc4cc(C(=O)NCCC(=O)O)ccc43)cc2)c(C)c1] | 3-((S)-1-((S)-1-((1s,4R)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)propanoic acid |
| 11b | 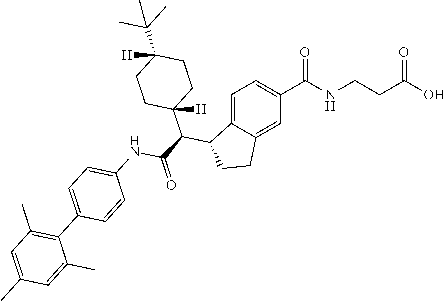 [SMILES: Cc1cc(C)c(-c2ccc(NC(=O)[C@@H]([C@H]3CC[C@@H](C(C)(C)C)CC3)[C@H]3CCc4cc(C(=O)NCCC(=O)O)ccc43)cc2)c(C)c1] | 3-((R)-1-((S)-1-((1s,4R)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)propanoic acid |
| 12a | 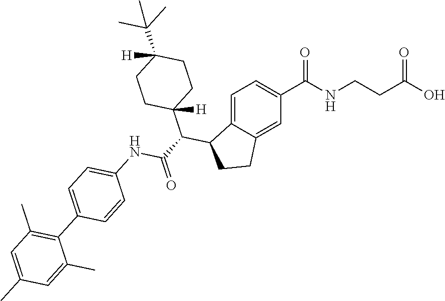 [SMILES: Cc1cc(C)c(-c2ccc(NC(=O)[C@H]([C@H]3CC[C@@H](C(C)(C)C)CC3)[C@@H]3CCc4cc(C(=O)NCCC(=O)O)ccc43)cc2)c(C)c1] | 3-((S)-1-((R)-1-((1s,4S)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)propanoic acid |
| 12b | 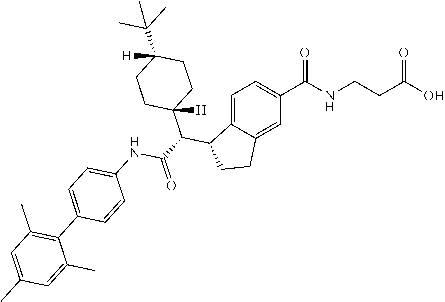 [SMILES: Cc1cc(C)c(-c2ccc(NC(=O)[C@H]([C@H]3CC[C@@H](C(C)(C)C)CC3)[C@H]3CCc4cc(C(=O)NCCC(=O)O)ccc43)cc2)c(C)c1] | 3-((R)-1-((R)-1-((1s,4S)-4-(tert- butyl)cyclohexyl)-2-oxo-2-((2′,4′,6′- trimethyl-[1,1′-biphenyl]-4- yl)amino)ethyl)-2,3-dihydro-1H-indene- 5-carboxamido)propanoic acid |
| 13a | 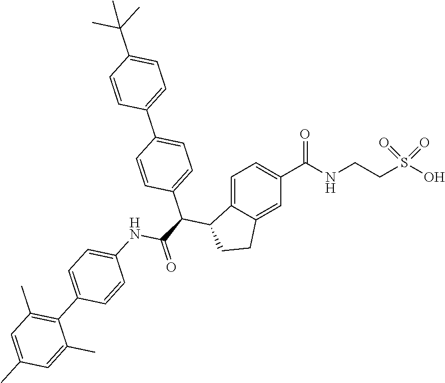 [SMILES: Cc1cc(C)c(-c2ccc(NC(=O)[C@@H](c3ccc(-c4ccc(C(C)(C)C)cc4)cc3)[C@H]3CCc4cc(C(=O)NCCS(=O)(=O)O)ccc43)cc2)c(C)c1] | 2-((R)-1-((R)-1-(4′-(tert-butyl)-[1,1′- biphenyl]-4-yl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 13b | 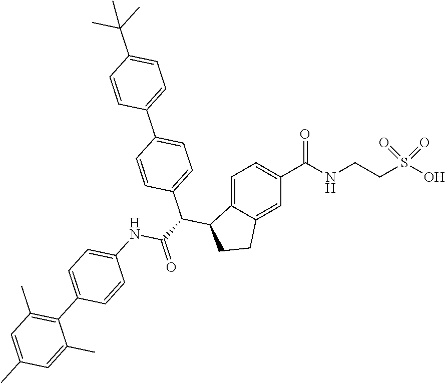 [SMILES: Cc1cc(C)c(-c2ccc(NC(=O)[C@H](c3ccc(-c4ccc(C(C)(C)C)cc4)cc3)[C@@H]3CCc4cc(C(=O)NCCS(=O)(=O)O)ccc43)cc2)c(C)c1] | 2-((S)-1-((S)-1-(4′-(tert-butyl)-[1,1′- biphenyl]-4-yl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 13c | 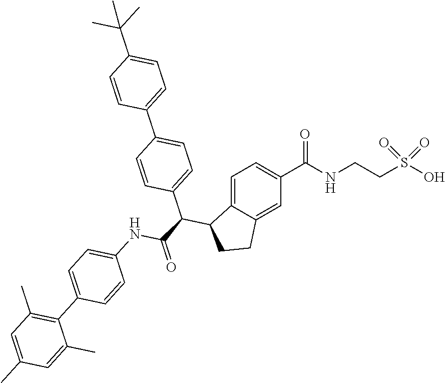 [SMILES: Cc1cc(C)c(-c2ccc(NC(=O)[C@@H](c3ccc(-c4ccc(C(C)(C)C)cc4)cc3)[C@@H]3CCc4cc(C(=O)NCCS(=O)(=O)O)ccc43)cc2)c(C)c1] | 2-((S)-1-((R)-1-(4′-(tert-butyl)-[1,1′- biphenyl]-4-yl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |
| 13d | 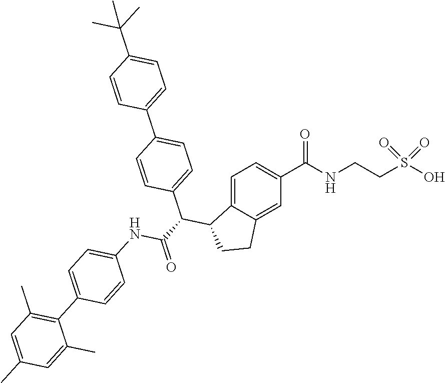 [SMILES: Cc1cc(C)c(-c2ccc(NC(=O)[C@H](c3ccc(-c4ccc(C(C)(C)C)cc4)cc3)[C@H]3CCc4cc(C(=O)NCCS(=O)(=O)O)ccc43)cc2)c(C)c1] | 2-((R)-1-((S)-1-(4′-(tert-butyl)-[1,1′- biphenyl]-4-yl)-2-oxo-2-((2′,4′,6′- trimethyl-[1,1′-biphenyl]-4- yl)amino)ethyl)-2,3-dihydro-1H-indene- 5-carboxamido)ethane-1-sulfonic acid |

### General Synthetic Methods

Certain processes for the manufacture of the compounds of this invention are provided as further features of the invention and are illustrated by the following exemplary reaction schemes. Those skilled in the art will appreciate that other synthetic routes may be used to synthesize the inventive compounds. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art. In particular, it is noted that the compounds prepared according to these Schemes may be modified further to provide new Examples within the scope of this invention. In addition, it will be evident from the detailed descriptions given in the Experimental section that the modes of preparation employed extend further than the general procedures described herein. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The starting materials are generally available from commercial sources such as Merck Sigma-Aldrich Inc. and Enamine Ltd. Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including Supplements (also available via the Beilstein online database).

As an initial note, in the preparation of compounds of the present invention, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparative methods and can be readily determined by one of ordinary skill in the art. The use of such protection/deprotection methods is also within the ordinary skill in the art. For a general description of protecting groups and their use, see T. W. Greene, Protective Groups in Organic Synthesis, John Wiley & Sons, New York, 1991 and Greene's Protective Groups inorganic Synthesis, John Wiley & Sons, New York 2006; also see March's Advanced Organic Chemistry: Reactions, Mechanisms, and Structure 8th Edition.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I compound.

### Pharmaceutical Dosages, Compositions and Formulations

The compounds or pharmaceutical compositions described herein that can be used in therapy can be formulated and dosages established in a fashion consistent with good medical practice taking into account the disorder to be treated, the condition of the individual patient, the site of delivery of the compound or pharmaceutical composition, the method of administration and other factors known to practitioners. The compounds or pharmaceutical compositions can be prepared according to the description of preparation described herein.

A therapeutically effective amount can be the amount of a compound or pharmaceutical composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment and can be ascertainable by one skilled in the art using known techniques.

One of ordinary skill in the art would understand that the amount, duration, and frequency of administration of a pharmaceutical composition or compound described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

Pharmaceutical compositions or compounds of the present disclosure can be administered to a subject in need thereof in a first administration, and in one or more additional administrations. The one or more additional administrations can be administered to the subject in need thereof minutes, hours, days, weeks, or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week, or more than once per month. The compounds or pharmaceutical compositions can be administered to the subject in need thereof in cycles of 21 days, 14 days, 10 days, 7 days, 4 days, or daily over a period of one to seven days.

In general, the compounds of this disclosure will be administered in a therapeutically effective amount by any of the accepted modes of administration for agents that serve similar utilities. Therapeutically effective amounts of compounds of Formula (I) may range from about 0.01 to about 500 mg/kg patient body weight per day, which can be administered in single or multiple doses. In one embodiment, the dosage level will be about 0.01 to about 250 mg/kg per day, about 0.05 to about 100 mg/kg per day, about 0.1 to about 250 mg/kg per day, about 0.1 to about 50 mg/kg per day or about 0.5 to about 100 mg/kg per day. In addition, the dosage can be about 0.05 to about 0.5, about 0.5 to about 5 or about 5 to about 50 mg/kg per day. For oral administration, the compositions may be provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the subject, the potency of the compound being utilized, the route and form of administration, and other factors.

These dosages are based on an average human subject having a weight of about 60 kg to 70 kg. The physician will readily be able to determine doses for subjects whose weight falls outside this range, such as infants and the elderly. Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian Subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding Such an active compound for the treatment of sensitivity in individuals.

Thus, one of skill in the art would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be deter mined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

In general, compounds of this disclosure will be administered as pharmaceutical compositions by any one of the following routes: oral, systemic (e.g., transdermal, intranasal or by suppository), or parenteral (e.g., intramuscular, intravenous, or subcutaneous) administration.

The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions. The compounds of the invention may also be administered topically, (intra)dermally, or transdermally to the skin or mucosa. Parenteral administration and administered parenterally as used herein includes modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Also provided, in some embodiments, is a pharmaceutical composition comprising the compound of Formula (I) and/or a pharmaceutically acceptable salt thereof and a pharmaceutically acceptable excipient.

A pharmaceutically acceptable excipient includes pharmaceutically acceptable material, composition, or vehicle, such as a liquid or solid filler, diluent, solvent or encapsulating material. Each excipient must be “acceptable” in the sense of being compatible with the other ingredients of the formulation and not injurious to the patient. Some examples of materials which can serve as pharmaceutically acceptable excipients include: (1) monosaccharides, disaccharides, and other carbohydrates including glucose, sucrose; lactose; mannose, trehalose, sorbitol or dextrins and (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide, aluminum hydroxide phosphate, citrate, and other organic acids; (15) alginic acid; (16) pyrogen-free water; (17) isotonic saline; (18) Ringer's solution; (19) ethyl alcohol; (20) antioxidants including ascorbic acid and methionine; preservatives, polypeptides; proteins, such as serum albumin; hydrophilic polymers; amino acids; chelating agents such as EDTA; salt-forming counter-ions such as sodium; metal complexes; and/or non-ionic surfactants or polyethylene glycol and other non-toxic compatible substances employed in pharmaceutical formulations.

Examples of excipients and their use may be found in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

Methods for formulation of the pharmaceutical compositions can include formulating any of the compounds described herein with one or more inert, pharmaceutically acceptable excipients (including carriers) to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically acceptable additives. This formulation can be an aqueous solution. Alternatively, the compositions described herein can be lyophilized or in powder form for re-constitution with a suitable vehicle, e.g., sterile pyrogen-free water, before use. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution, or emulsion in oily or aqueous vehicles. Suitable oily vehicles can include, but are not limited to, lipophilic solvents or vehicles such as fatty oils, synthetic fatty acid esters, or liposomes. Aqueous injection suspensions can contain substances which increase the viscosity of the suspension. The suspension can also contain suitable stabilizers. Injections can be formulated for bolus injection or continuous infusion.

For parenteral administration, the compounds can be formulated in a unit dosage injectable form (e.g., solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

The pharmaceutical compositions provided herein may be formulated as immediate or modified release dosage forms, including delayed-, sustained, pulsed-, controlled, targeted-, and programmed-release forms. Thus, in some preferred embodiments, the active ingredient(s) is administered in a pharmaceutical composition which is an immediate release oral dosage form, preferably but not necessarily including an enteric coating. In some preferred embodiments, the active ingredients(s) are administered in a pharmaceutical composition which is an extended-release oral dosage form, preferably but not necessarily including an enteric coating. In further preferred embodiments, the active ingredients are administered in a pharmaceutical composition which contains both an immediate release dose and an extended-release dose or pulsed release dose of the first agent preferably but not necessarily also including an enteric coating. Such dual release dosage forms achieve release of an initial dose of active ingredient, followed late in time by another pulsed release, or by a sustained release dose. Methodologies for preparing such dual release dosage forms are well known in the art.

In some embodiments, the active ingredients are formulated into a controlled release matrix tablet, which contains one or more polymeric matrix materials that promote the sustained, delayed or pulsed release profile. Non-limiting examples of such polymeric matrix materials include cellulosic materials as described above, and carbomers, for example those sold by Lubrizol Corporation under the name Carbopol®, for example Carbopol® 71G NF, Carbopol® 971P NF and Carbopol® 974P NF polymers.

Some preferred examples of extended-release compositions suitable for use in the methods and compositions of the invention include, for example and not limitation, extended-release compositions found in nifedipine formulations such as Adalat CC@, Procardia® XL, Afeditab® CR and Nifedical® XL; and in diltiazem formulations such as Cardizem® CD, Cardizem® LA, Cardizem® SR, Cartia® XT and Dilacor® XR.

### Combinations

Compounds of the present disclosure may be used in methods of treating in combination with one or more other combination agents (e.g., one, two, or three other drugs) that are used in the prevention, treatment, control, amelioration, or reduction of risk of the diseases or conditions for which compounds of the present disclosure are useful. In some embodiments, the combination of the drugs together are safer or more effective than either drug alone. In some embodiments the compound disclosed herein and the one or more combination agents have complementary activities that do not adversely affect each other. Such molecules can be present in combination in amounts that are effective for the purpose intended. Such other drug(s) may be administered, by a route and in an amount commonly used therefore, contemporaneously or sequentially with a compound of the present disclosure. When a compound of the present disclosure is used contemporaneously with one or more other drugs, in some embodiments, the agents are administered together in a single pharmaceutical composition in unit dosage form.

Examples of combination agents are GLP-1 drugs. Any GLP-1 inhibitor can be used as the second agent in combination with a compound of the present invention. A “GLP-1 receptor agonist” refers to compounds having GLP-1 receptor activity. Such exemplary compounds include exendins, exendin analogs, exendin agonists, GLP-1(7-37), GLP-1(7-37) analogs, GLP-1(7-37) agonists, and the like. The GLP-1 receptor agonist compounds may optionally be amidated.

The term “exendin” includes naturally occurring (or synthetic versions of naturally occurring) exendin peptides that are found in the salivary secretions of the Gila monster. Exendins of particular interest include exendin-3 and exendin-4. The exendins, exendin analogs, and exendin. agonists for use in the methods described herein may optionally be amidated, and may also be in an acid form, pharmaceutically acceptable salt form, or any other physiologically active form of the molecule.

The combination of a GLP-1 and a GIPR antagonist may achieve the effect of simultaneously improving insulin resistance and reducing excessive fat accumulation (obesity), while lowering blood glucose, and also interfering with lipid metabolism. In this regard, the GLP-1 part may be used to improve glucose tolerance, reduces appetite, lower blood glucose and reduce body weight; whereas the GIPR antagonist compound may be used to reduce the further accumulation of fat, impact reduction in fat mass vs. lean body mass, and improve liver function. The fat reduction effect of the GIPR antagonist and the weight loss effect of the GLP-1 may be used synergistically to treat the non-alcoholic fatty liver disease/non-alcoholic steatohepatitis. This disclosure provides a drug that will benefit patients who have one or more diseases of non-alcoholic fatty liver disease/nonalcoholic steatohepatitis, type 2 diabetes and obesity.

In one embodiment, the GLP-1 receptor agonist is selected from the group consisting of exenatide, liraglutide, lixisenatide, albiglutide, dulaglutide, semaglutide, efpeglenatide, taspoglutide. danuglipron, orforglipron, lotiglipron, PF-06954522, HM15211, L Y3298176, Medi-0382, NN-9924, TTP-054, TTP-273, CT-996, ECC5004, XW004, XW014, MDR-001, ZT002, KN-056, GL0034, GSBR-1290, noiiglutide, RGT-075, TTP-273, HRS-7535, GMA-105, TG103, GZR-18, GX-G6, ecnoglutide, PB-119, QLG2065, beinaglutide, those described in WO2018109607, those described in WO2019239319 (PCT/IB2019/054867 filed Jun. 11, 2019), and those described in WO2019239371 (PCT/IB2019/054961 filed Jun. 13, 2019).

In one embodiment, the molar ratio of a GLP-1 receptor agonist to a GIPR antagonist is from about 1:1 to 1:110, 1:1 to 1:100, 1:1 to 1:75, 1:1 to 1:50, 1:1 to 1:25, 1:1 to 1:10, 1:1 to 1:5, and 1:1. In one embodiment, the molar ratio of a GIPR antagonist to a GLP-1 receptor agonist is from about 1:1 to 1:110, 1:1 to 1:100, 1:1 to 1:75, 1:1 to 1:50, 1:1 to 1:25, 1:1 to 1:10, and 1:1 to 1:5.

In one embodiment, the therapeutically effective amounts of the GLP-1 receptor agonist and the GIPR antagonist are administered to the subject simultaneously.

In one embodiment, the therapeutically effective amounts of the GLP-1 receptor agonist and the GIPR antagonist are administered to the subject sequentially.

Any GCGR agonist can be used as the second agent in combination with a compound of the present invention. The combination of a GCGR agonist and a GIPR antagonist may achieve the effect of simultaneously improving insulin resistance and reducing excessive fat accumulation (obesity), while lowering blood glucose, and also interfering with lipid metabolism. Examples of GCGR agonists include antibodies disclosed in U.S. Pat. No. 11,845,802.

In one embodiment, the molar ratio of a GCGR receptor agonist to a GIPR antagonist is from about 1:1 to 1:110, 1:1 to 1:100, 1:1 to 1:75, 1:1 to 1:50, 1:1 to 1:25, 1:1 to 1:10, 1:1 to 1:5, and 1:1. In one embodiment, the molar ratio of a GIPR antagonist to a GCGR receptor agonist is from about 1:1 to 1:110, 1:1 to 1:100, 1:1 to 1:75, 1:1 to 1:50, 1:1 to 1:25, 1:1 to 1:10, and 1:1 to 1:5.

In one embodiment, the therapeutically effective amounts of the GCGR receptor agonist and the GIPR antagonist are administered to the subject simultaneously.

In one embodiment, the therapeutically effective amounts of the GCGR receptor agonist and the GIPR antagonist are administered to the subject sequentially.

The addition of a third hormone to incretin-based therapies, glucagon GCGR agonism has been shown to significantly increase energy expenditure. Thus, the GCGR agonist can be used in combination with the GIPR antagonist and the GLP-1 receptor agonist (GIPR/GLP-1R/GCGR triple agents) in triple therapy. Examples of triple therapy agents include NN9423 (NovoNordisk) and Lilly's LY3437943. [Expert Opinion on Drug Discovery 2023, VOL. 18, NO. 6, 659-669.

Due to the compounds of the invention action on metabolic diseases the compounds may also be beneficially used in combination with other agents such as anti-diabetic agents.

Suitable anti-diabetic agents include insulin, metformin, GLP-1 receptor agonists (described herein above), SGL T2 inhibitors, monoacylglycerol Oacyltransferase inhibitors, phosphodiesterase (PDE)-10 inhibitors, AMPK activators [e.g., ETC-1002 (bempedoic acid)], sulfonylureas (e.g., acetohexamide, chlorpropamide, diabinese, glibenclamide, glipizide, glyburide, glimepiride, gliclazide, glipentide, gliquidone, glisolamide, tolazamide, and tolbutamide), meglitinides, α-amylase inhibitors (e.g., tendamistat, trestatin and AL-3688), an α-glucoside hydrolase inhibitor (e.g., acarbose), α-glucosidase inhibitors (e.g., adiposine, camiglibose, emiglitate, miglitol, voglibose, pradimicin-Q, and salbostatin), PPARy agonists (e.g., balaglitazone, ciglitazone, darglitazone, englitazone, isaglitazone, pioglitazone and rosiglitazone), PPAR a/y agonists (e.g., CLX-0940, GW-1536, GW-1929, GW-2433, KRP—297, L-796449, LR-90, MK-0767 and SB-219994), protein tyrosine phosphatase-1 B (PTP-1 B) inhibitors [e.g., trodusquemine, hyrtiosal extract, and compounds disclosed by Zhang, S. et al., Drug Discovery Today, 12(9/10), 373-381 (2007)], SIRT-1 activators (e.g., resveratrol, GSK2245840 or GSK184072), dipeptidyl peptidase IV (OPP—IV) inhibitors (e.g., those in WO2005116014, sitagliptin, vildagliptin, alogliptin, dutogliptin, linagliptin and saxagliptin), insulin secretagogues, fatty acid oxidation inhibitors, A2 antagonists, c-jun amino-terminal kinase (JNK) inhibitors, glucokinase activators (GKa) such as those described in WO2010103437, WO2010103438, WO2010013161, WO2007122482, TTP-399, TTP-355, TTP-547, AZD1656, ARRY403, MK-0599, TAK-329, AZD5658 or GKM-001, insulin, insulin mimetics, glycogen phosphorylase inhibitors (e.g., GSK1362885), VPAC2 receptor agonists, glucagon receptor modulators such as those described in Demong, D. E. et al., Annual Reports in Medicinal Chemistry 2008, 43, 119-137, GPR119 modulators, particularly agonists, such as those described in WO2010140092, WO2010128425, WO2010128414, WO2010106457, Jones, R. M. et al., Annual Reports in Medicinal Chemistry 2009, 44, 149-170 (e.g., MBX-2982, GSK1292263, APD597 and PSN821), FGF21 derivatives or analogs such as those described in Kharitonenkov, A. et al., Current Opinion in Investigational Drugs 2009, 10(4)359-364, TGR5 (also termed GPBAR1) receptor modulators, particularly agonists, such as those described in Zhong, M., Current Topics in Medicinal Chemistry, 2010, 10(4), 386-396 and INT777, GPR40 agonists, such as those described in Medina, J. C., Annual Reports in Medicinal Chemistry, 2008, 43, 75-85, including but not limited to TAK-875, GPR120 modulators, particularly agonists, high-affinity nicotinic acid receptor (HM74A) activators, and SGL T1 inhibitors, such as GSK1614235. A further representative listing of anti-diabetic agents that can be combined with the compounds of the present invention can be found, for example, at page 28, line 35 through page 30, line 19 of WO2011005611.

Due to the compounds of the invention action on metabolic diseases the compounds may also be beneficially used in combination with other agents. Other combination agents include amylin, NPY2 receptor agents, cannabinoid receptor agents, serotonin receptor agents and MC4R receptor agents which are useful for the treatment of obesity.

In addition, other agents such as lipid modulating agents (HMG-CoA reductase inhibitors such as atorvastatin, pravastatin, pitavastatin, lovastatin, simvastatin, fluvastatin, NK-104 (a.k.a. itavastatin, or nisvastatin or nisbastatin) and ZD-4522 (a.k.a. rosuvastatin, or atavastatin or visastatin) are useful as combination agents.

Due to the compounds of the invention action on metabolic diseases the compounds may also be beneficially used in combination with other cardiovascular agents such as anti-hypertensive agents. Any anti-hypertensive agent can be used as the second agent in such combinations. Such antihypertensive activity is readily determined by those skilled in the art according to standard assays (e.g., blood pressure measurements). Examples include calcium channel blockers (amlodipine besylate), ACE-inhibitors, angiotensin II receptor antagonists, beta-adrenergic receptor blockers, alpha-adrenergic receptor blockers, peripheral vasodilators and diuretics.

Accordingly, the pharmaceutical compositions of the present disclosure also include those that contain one or more other active ingredients, in addition to a compound of the present disclosure. The weight ratio of the compound of the present disclosure to the second active agent may be varied and will depend upon the effective dose of each ingredient. Generally, an effective dose of each will be used. In some embodiments, combination therapy includes therapies in which the compound of the present disclosure and one or more other drugs are administered separately, and in some cases, the two or more agents are administered on different, overlapping schedules. It is also contemplated that when used in combination with one or more other active ingredients, the compounds of the present disclosure and the other active ingredients may be used in lower doses than when each is used singly. In some embodiments, the combination agent is a drug for reduction of symptoms of obesity.

As the present invention has an aspect that relates to the treatment of the disease/conditions described herein with a combination of active ingredients which may be administered jointly, the invention also relates to combining separate pharmaceutical compositions in a single dosage form, such as (but not limited to) a single tablet or capsule, a bilayer or multilayer tablet or capsule, or through the use of segregated components or compartments within a tablet or capsule.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of Formula I a prodrug thereof or a salt of Such compound or prodrug and a second compound as described above. The kit comprises a means for containing the separate compositions such as a container, a divided bottle or a divided foil packet. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. Second Week, Monday, Tuesday, . . . etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules and vice versa. The memory aid should reflect this. In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered microchip memory coupled with a liquid crystal readout, or audible reminder signal which, for example, reads out the date that the last daily dose has been taken and/or reminds one when the next dose is to be taken.

The compounds, pharmaceutical compositions, and methods of the present disclosure can be useful for treating a subject such as, but not limited to, a mammal, a human, a non-human mammal, a domesticated animal (e.g., laboratory animals, household pets, or livestock), a non-domesticated animal (e.g., wildlife), a dog, a cat, a rodent, a mouse, a hamster, a cow, a bird, a chicken, a fish, a pig, a horse, a goat, a sheep, or a rabbit. In preferred embodiments, compounds, pharmaceutical compositions, and methods of the present disclosure are used for treating a human; male or female.

The present invention further comprises use of a compound of Formula I for use as a medicament (Such as a unit dosage tablet or unit dosage capsule). In another embodiment, the present invention comprises the use of a compound of Formula I for the manufacture of a medicament (such as a unit dosage tablet or unit dosage capsule) to treat one or more of the conditions discussed herein.

In practicing the methods described herein, therapeutically effective amounts of the compounds or pharmaceutical compositions described herein can be administered to a subject in need thereof, often for treating and/or preventing a condition or progression thereof. The compounds of this invention and pharmaceutical compositions thereof can affect the physiology of the subject, such as the metabolic system, the immune system, inflammatory response, or other physiologic affect. In addition, the compounds of this invention and pharmaceutical compositions thereof can affect the neuronal (nervous system) based on the incretins role in addictive behavior, Alzheimer's and Parkinson's. A therapeutically effective amount can vary widely depending on the severity of the disease, the age and relative health of the subject, the potency of the compounds used, and other factors.

The Formula I compounds of this invention, their prodrugs and the salts of such compounds and prodrugs (including combinations thereof) are all adapted to therapeutic use as agents that mediate the GIPR in mammals, particularly humans e.g., males, females. For example, these compounds act as GIPR receptor antagonists and thus are useful for the treatment of the various conditions (e.g., those described herein) in which such action is implicated.

Given the positive correlation between mediation of the GIPR receptor with the number of physiological effects in tissues (including promotion of fat storage in adipocytes and promotion of pancreatic islet ˜-cell function and glucose-dependent insulin secretion and also that GIPR is highly expressed in a number of tissues, including the pancreas, gut, adipose tissue, heart, pituitary, adrenal cortex, and brain (Usdin et al., Endocrinology. 1993, 133:2861-2870)), GIPR antagonists are useful for the treatment of glucose metabolism (e.g. Type 2 diabetes, elevated glucose levels, elevated insulin levels, dyslipidemia, metabolic syndrome (Syndrome X or insulin resistance syndrome), glucosuria, metabolic acidosis, Type 1 diabetes, obesity and conditions exacerbated by obesity), pancreatitis, cirrhosis and Turner's & Cushing's syndrome.

Preferred disease/conditions include obesity and type II diabetes. An especially preferred disease/condition is obesity.

Through the compounds of this invention capacity to treat obesity they also have a treatment impact on cardiovascular diseases, including coronary artery disease, myocardial infarction, heart failure. stroke, venous thromboembolic disease, and pulmonary hypertension. Circulation 2021 143:e984-e1010

Through the compounds of this invention capacity to treat obesity they also have a treatment impact on cancer including post-menopausal breast, colorectal, endometrial, esophageal, pancreatic, renal, liver, stomach, gallbladder, ovarian, thyroid, multiple myeloma, and meningioma. Other cancers associated with obesity include cancers of the mouth, pharynx and larynx, prostate, and male breast, as well as diffuse large B-cell lymphoma. A high risk associated with obesity is shown for three of the most challenging cancers to treat-pancreatic, esophageal, and gallbladder—as well as the two most prevalent malignancies-breast and colorectal cancer. Cancers 2023, 15,485

Through the compounds of this invention capacity to treat obesity they also have a treatment impact on obstructive sleep apnea, and associated conditions.

Through the compounds of this invention capacity to treat obesity they also have a treatment impact on osteoarthritis and associated disease/conditions.

Further the compounds of this invention can be used to treat any GIPR-related condition, disease, or disorder including the following: diabetes [e.g. Type 1 diabetes mellitus (T1 D), Type 2 diabetes mellitus (T2DM), including pre-diabetes], idiopathic T1 D (Type 1 b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, depression, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

### EXAMPLES

Compounds of Formula (I) and sub formulae and species described herein, including those where the substituent groups as defined herein, can be prepared as illustrated and described below. Thus, the following preparations of compounds of Formula (I) and intermediates are given to enable those skilled in the art to more clearly understand and to practice the present disclosure. The Examples should not be considered as limiting the scope of the disclosure, but merely as being illustrative and representative thereof.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These Examples are merely illustrative of some methods by which the compounds of this disclosure can be synthesized, and various modifications to these Examples can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless otherwise noted, all reagents were used without further purification. ¹H NMR spectra were obtained in CDCl₃, DMSO-d₆, or CD₃OD, unless stated otherwise, at room temperature on a Bruker AVANCE III HD 300 MHz, Bruker AVANCE III HD 400 MHz, or AVANCE NEO 400 MHz instrument or an NMR spectrometer of similar caliber. When more than one conformer was detected, the chemical shifts for the most abundant one is reported. Chemical shifts of ¹H NMR spectra were recorded in parts per million (ppm) on the 6 scale from an internal standard of residual solvent. Splitting patterns are designed as s, singlet; d, doublet; t, triplet; q, quartet; m, multiplet; br, broad.

The following abbreviations are used in the text: LCMS=liquid chromatography-mass spectrometry, ESI=Electrospray Ionization, M+H=a unit higher than the monoisotopic mass of the uncharged molecule, HPLC=High pressure liquid chromatography, Prep-HPLC=preparatory scale HPLC, AcOH=acetic acid, ACN or MeCN=acetonitrile, PE=petroleum ether, EA or EtOAc=ethyl acetate, BSA=Bovine Serum Albumin, DIEA=N,N-diisopropylethylamine, DEA=diethanolamine, DMSO=dimethyl sulfoxide, DMF=N, N-dimethylacetamide, DCM=dichloromethane, dppf=1,1′-Bis(diphenylphosphino)ferrocene, EtOH=ethanol, FA=formic acid, MeOH=methanol, MtBE/MTBE=methyl tert-butyl ether, TEA or Et₃N=triethylamine, GOI=genes of interest, HATU=Hexafluorophosphate Azabenzotriazole Tetramethyl Uronium, HCl=hydrochloric acid, hERG=human ether-a-go-go-related gene, HBSS=Hanks' Balanced Salt Solution, HEPES=4-(2-hydroxyethyl)-1-piperazineethane sulfonic acid, SFC=Supercritical fluid chromatography, THF=tetrahydrofuran, TLC=thin layer chromatography, TCFH=Chloro-N,N,N′,N′-tetramethylformamidinium Hexafluorophosphate, t-BuOH=tert-butyl alcohol, t-BuONa=sodium tert-butyloxide, Oac=acetate, NMI=1-Methylimidazole, POCl₃=Phosphoryl chloride, POBr₃=Phosphoryl chloride, py=pyridine, Pd/C=palladium on carbon, HTRF—Homogeneous Time-Resolved Fluorescence, RT=retention time, h=hour, hrs=hours, aq.=aqueous, min.=minute, sat.=saturated, equiv=equivalent, e.e.=enantiomeric excess, UV=ultraviolet, Ns=2-nitrobenzene-1-sulfonyl.

Unless otherwise indicated, the chemical names used herein are generated using the Chemdraw Professional Version 22.2.0.3348 software naming programs.

### Synthesis and Characterization Examples

For certain compounds disclosed herein the absolute stereochemistry has not been independently confirmed. Thus, in some instances, the absolute stereo configurations of one or more chiral centers are arbitrarily assigned (e.g., stereochemistry of one chiral center is known and remaining chiral centers arbitrarily assigned). Accordingly, the enantiomers or diastereomers are identified by their respective properties, for example, retention times on a prep chiral HPLC, chiral SFC, NMR shift or optical rotation or its biological activities (e.g., as described further in the Examples). Thus, should the stereochemistry assigned to any compound or compounds ultimately be proven incorrect, then the analytical data (e.g., prep chiral HPLC, chiral SFC, NMR shift or optical rotation or biological activity) associated with each compound is determinative of the actual identity of the compound. In addition, in light of such corrected stereochemical designation appropriate adjustments to the stereochemistry identification contained in the description, examples, tables and claims should be adjusted as needed by one skilled in the art.

Chiral analytical separation methods (e.g., supercritical fluid chromatography (SFC), and high performance liquid chromatography (HPLC)) used in the following synthetic examples are summarized in Table A below. These methods were used to identify a single compound/stereoisomer from a mixture of chiral compounds based on a peak retention time.

TABLE A

| | | | | | | |
|---|---|---|---|---|---|---|
| | | | | | Column | Back |
| Method | Column | Mobile Phase | Flow Type | Flow Rate | Temp. | Pressure |

| | | | | | |
|---|---|---|---|---|---|
| A | CHIRAL | A: MTBE:Hex | Isocratic: | 1.67 | Ambient |
| | Cellulose-SB, | 1:1 (0.1% TFA) | 10% B | mL/min | |
| | 0.46 × 5 cm, 3 μm | B: MeOH | | | |
| B | CHIRAL NX (2) | A: Hex (0.1% | Isocratic: | 1.67 | Ambient |
| | 0.46 × 5 cm, 3 μm | DEA) B: EtOH | 30% B | mL/min | |
| C | CHIRAL | A: MTBE | Isocratic: | 1.00 | Ambient |
| | Cellulose-SB, | (0.1% TFA) B: | 10% B | mL/min | |
| | 0.46 × 5 cm, 3 μm | EtOH | | | |
| D | CHIRALPAK IE- | A: MTBE | Isocratic: | 1.67 | Ambient |
| | 3 | (0.1% DEA) B: | 20% B | mL/min | |
| | 0.46 × 5 cm, 3 μm | MeOH | | | |

### Stereochemical Analysis of Intermediates and Examples:

For compounds that have indicated stereochemistry, unless otherwise noted in the experimental procedures, the stereochemistry has been arbitrarily assigned. The relative stereochemistry of the cyclohexyl ring on Intermediate B was determined to be the trans configuration based on analogy to known literature 1H-NMR data (See WO2022/073904 and J. Med. Chem. 2007, 50, 113-128).

### Intermediate B: methyl 3-(1-(((1r,4r)-4-(tert-butyl)cyclohexyl)amino)-2,3-dihydro-1H-indene-5-carboxamido)propanoate

### Step 1: butyl 1-oxo-2,3-dihydro-1H-indene-5-carboxylate

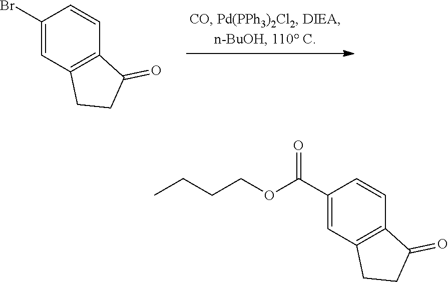 [SMILES: O=C1CCc2cc(Br)ccc21 ; CCCCOC(=O)c1ccc2c(c1)CCC2=O]

A solution of 5-bromo-2,3-dihydroinden-1-one (8.0 g, 37.90 mmol), dichlorobis(triphenylphosphine)palladium(II) (2.66 g, 3.79 mmol) and DIEA (14.70 g, 113.71 mmol) in 1-butanol (80 mL) was stirred at 110° C. for 3 hours under CO atmosphere. The resulting mixture was concentrated under reduced pressure. The crude residue was purified by silica gel column chromatography, eluted with PE/EA (0-35%) to afford butyl 1-oxo-2,3-dihydro-1H-indene-5-carboxylate (6.0 g, crude) as a black oil. LCMS (ESI) [M+H]+: 233.

### Step 2: butyl 1-(((1r,4r)-4-(tert-butyl)cyclohexyl)amino)-2,3-dihydro-1H-indene-5-carboxylate

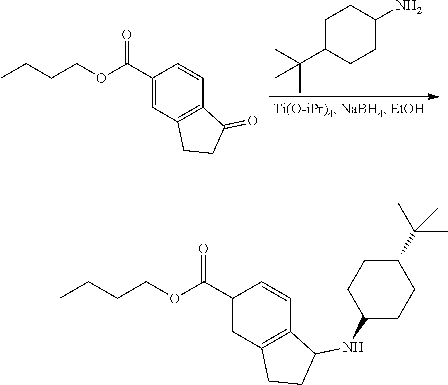

To a stirred solution of butyl 1-oxo-2,3-dihydro-1H-indene-5-carboxylate (6.0 g, 25.83 mmol), 4-tert-butylcyclohexan-1-amine (4.81 g, 31.0 mmol) and titanium isopropoxide (14.68 g, 51.66 mmol) in EtOH (60 mL) was added NaBH₄ (1.95 g, 51.66 mmol) in portions at 0° C. The resulting mixture was warmed to 25° C. and stirred for an additional 1 hour. The reaction was quenched by the addition of H₂O (20 mL) at 0° C. The resulting mixture was extracted with EA (3×200 mL). The combined organic layers were washed with H₂O (1×100 mL) and dried over anhydrous Na₂SO₄, filtered and concentrated under reduced pressure. The crude residue was purified by silica gel column chromatography, eluted with PE/EA (0-40%) to afford butyl 1-(((1r,4r)-4-(tert-butyl)cyclohexyl)amino)-2,3-dihydro-1H-indene-5-carboxylate (3.0 g) as a black oil. LCMS (ESI) [M+H]⁺: 372.

### Step 3: 1-(((1r,4r)-4-(tert-butyl)cyclohexyl)amino)-2,3-dihydro-1H-indene-5-carboxylic acid

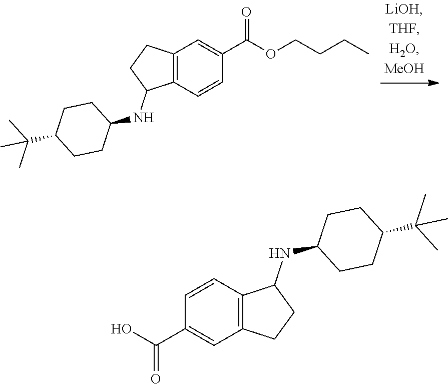 [SMILES: CCCCOC(=O)c1ccc2c(c1)CCC2N[C@H]1CC[C@H](C(C)(C)C)CC1 ; CC(C)(C)[C@H]1CC[C@H](NC2CCc3cc(C(=O)O)ccc32)CC1]

A solution of butyl 1-(((1r,4r)-4-(tert-butyl)cyclohexyl)amino)-2,3-dihydro-1H-indene-5-carboxylate (3 g, 8.07 mmol) and LiOH monohydrate (0.39 g, 16.15 mmol) in THF (10 mL), MeOH (10 mL) and H₂O (10 mL) was stirred at 50° C. for 2 hours. Upon completion, the mixture was acidified to pH=2 with HCl (1 M in water). The resulting mixture was extracted with EA (3×100 mL). The combined organic layers were washed with H₂O (1×100 mL) and dried over anhydrous Na₂SO₄, filtered and concentrated under reduced pressure to give crude 1-(((1r,4r)-4-(tert-butyl)cyclohexyl)amino)-2,3-dihydro-1H-indene-5-carboxylic acid (2.0 g, crude) as a yellow oil, which was used for next step without further purification. LCMS (ESI) [M+H]⁺: 316.

### Step 4: methyl 3-(1-(((1r,4r)-4-(tert-butyl)cyclohexyl)amino)-2,3-dihydro-1H-indene-5-carboxamido)propanoate

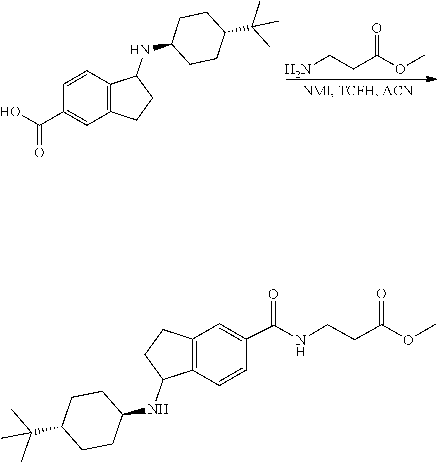

A solution of 1-(((1r,4r)-4-(tert-butyl)cyclohexyl)amino)-2,3-dihydro-1H-indene-5-carboxylic acid (2.0 g, 6.34 mmol), methyl 3-aminopropanoate (2.76 g, 19.02 mmol), TCFH (3.56 g, 12.68 mmol) and NMI (1.04 g, 12.68 mmol) in acetonitrile (20 mL) was stirred at 25° C. for 2 hours. Upon completion, the resulting mixture was concentrated under reduced pressure. The crude residue was purified by silica gel column chromatography, eluted with PE/EA (0-50%) to afford methyl 3-(1-(((1r,4r)-4-(tert-butyl)cyclohexyl)amino)-2,3-dihydro-1H-indene-5-carboxamido)propanoate (Intermediate B; 1.2 g) as a brown oil. LCMS (ESI) [M+H]⁺: 443.

### Intermediate C: methyl 3-(1-((4-cyclohexylphenyl)amino)-2,3-dihydro-1H-indene-5-carboxamido)propanoate

### Step 1: butyl 1-((4-cyclohexylphenyl)amino)-2,3-dihydro-1H-indene-5-carboxylate

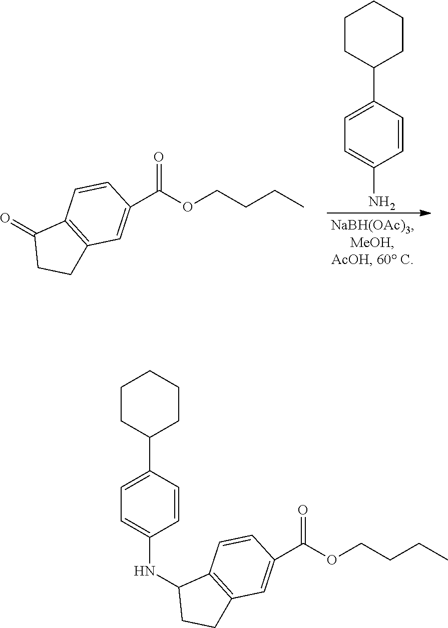 [SMILES: CCCCOC(=O)c1ccc2c(c1)CCC2=O ; Nc1ccc(C2CCCCC2)cc1 ; CCCCOC(=O)c1ccc2c(c1)CCC2Nc1ccc(C2CCCCC2)cc1]

NaBH₃CN (0.54 g, 8.61 mmol) was added in portions to a stirred solution of butyl 1-oxo-2,3-dihydroindene-5-carboxylate (1 g, 4.31 mmol), acetic acid (5 mL) and 4-cyclohexylaniline (1.51 g, 8.61 mmol) in MeOH (10 mL) at 0° C. The resulting mixture was then stirred at 60° C. for an additional 1 hour. The reaction was quenched with H₂O (100 mL) at 0° C. The resulting mixture was extracted with EA (3×200 mL). The combined organic layers were washed with H₂O (1×200 mL), dried over anhydrous Na₂SO₄, filtered and concentrated under reduced pressure. The crude residue was purified by silica gel column chromatography, eluted with PE/EA (0-40%) to afford butyl 1-((4-cyclohexylphenyl)amino)-2,3-dihydro-1H-indene-5-carboxylate (600 mg) as a yellow oil. LCMS (ESI) [M+H]⁺: 392.

### Step 2: 1-((4-cyclohexylphenyl)amino)-2,3-dihydro-1H-indene-5-carboxylic acid

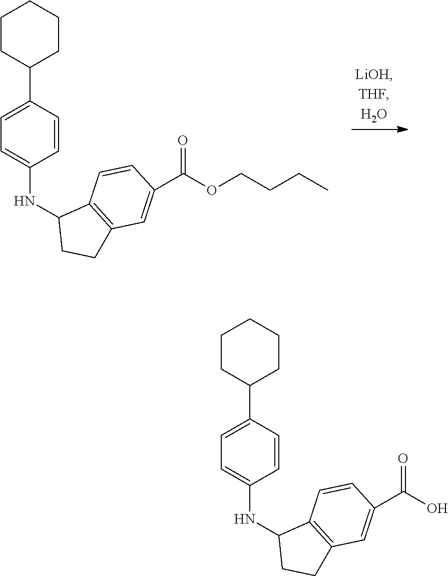 [SMILES: CCCCOC(=O)c1ccc2c(c1)CCC2Nc1ccc(C2CCCCC2)cc1 ; O=C(O)c1ccc2c(c1)CCC2Nc1ccc(C2CCCCC2)cc1]

A solution of butyl 1-((4-cyclohexylphenyl)amino)-2,3-dihydro-1H-indene-5-carboxylate (600 mg, 1.53 mmol) and LiOH monohydrate (73 mg, 3.06 mmol) in THF (2 mL) and H₂O (2 mL) was stirred at 25° C. for 2 hours. Upon completion, the mixture was diluted with water (100 mL) and acidified to pH=2 with HCl (1 M in water). The resulting mixture was extracted with EA (3×100 mL). The combined organic layers were washed with H₂O (1×100 mL), dried over anhydrous Na₂SO₄, filtered and concentrated under reduced pressure. The crude residue was purified by silica gel column chromatography, eluted with PE/EA (0-50%) to afford 1-((4-cyclohexylphenyl)amino)-2,3-dihydro-1H-indene-5-carboxylic acid (200 mg) as a yellow oil. LCMS (ESI) [M+H]⁺: 336.

### Step 3: methyl 3-(1-((4-cyclohexylphenyl)amino)-2,3-dihydro-1H-indene-5-carboxamido)propanoate

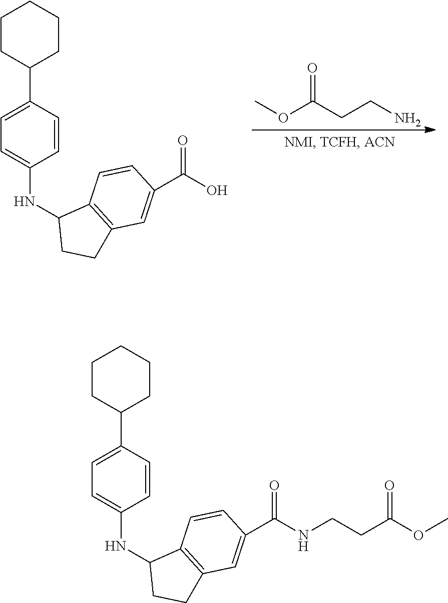

A solution of 1-((4-cyclohexylphenyl)amino)-2,3-dihydro-1H-indene-5-carboxylic acid (200 mg, 0.596 mmol), methyl 3-aminopropanoate (123 mg, 1.19 mmol), TCFH (335 mg, 1.19 mmol) and NMI (98 mg, 1.19 mmol) in acetonitrile (2 mL) was stirred at 25° C. for 1 hour. Upon completion, the resulting mixture was concentrated under reduced pressure. The crude residue was purified by silica gel column chromatography, eluted with DCM/MeOH (0-7%) to afford methyl 3-(1-((4-cyclohexylphenyl)amino)-2,3-dihydro-1H-indene-5-carboxamido)propanoate (150 mg) as a yellow oil. LCMS (ESI) [M+H]⁺: 421.

### Synthesis of intermediates D, E, F and G: 1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid, 1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid, 1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid and 1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid

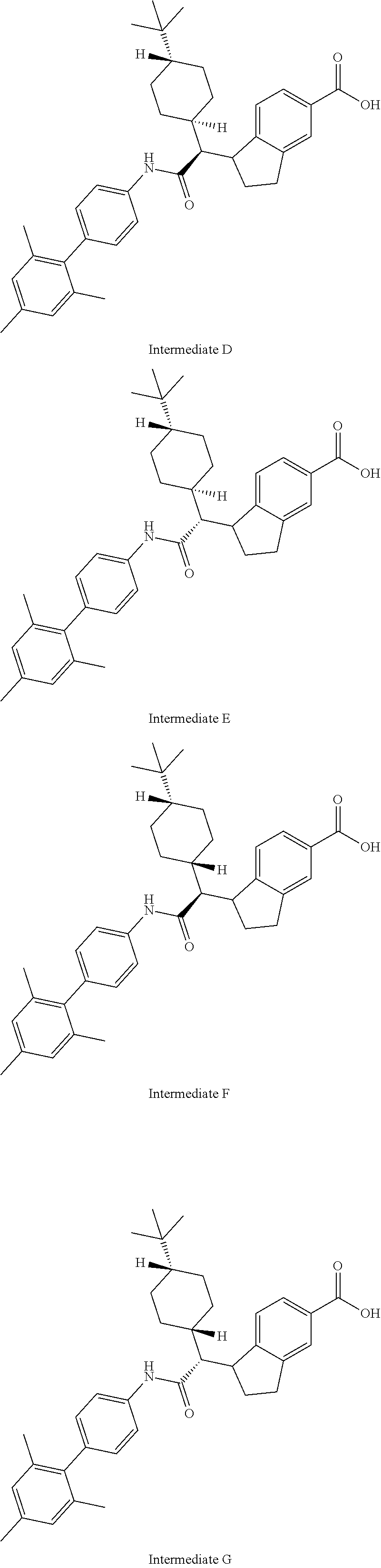 [SMILES: Cc1cc(C)c(-c2ccc(NC(=O)[C@H](C3CCc4cc(C(=O)O)ccc43)[C@H]3CC[C@H](C(C)(C)C)CC3)cc2)c(C)c1 ; Cc1cc(C)c(-c2ccc(NC(=O)[C@@H](C3CCc4cc(C(=O)O)ccc43)[C@H]3CC[C@H](C(C)(C)C)CC3)cc2)c(C)c1 ; Cc1cc(C)c(-c2ccc(NC(=O)[C@H](C3CCc4cc(C(=O)O)ccc43)[C@H]3CC[C@@H](C(C)(C)C)CC3)cc2)c(C)c1 ; Cc1cc(C)c(-c2ccc(NC(=O)[C@@H](C3CCc4cc(C(=O)O)ccc43)[C@H]3CC[C@@H](C(C)(C)C)CC3)cc2)c(C)c1]

### Step 1: methyl 2-(4-(tert-butyl)cyclohexylidene)acetate

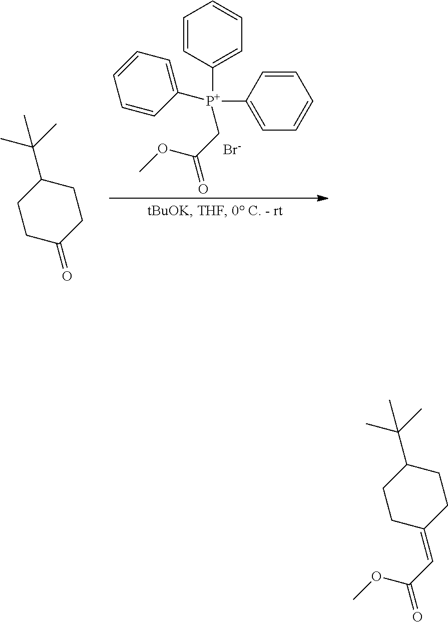 [SMILES: CC(C)(C)C1CCC(=O)CC1 ; COC(=O)C[P+](c1ccccc1)(c1ccccc1)c1ccccc1 ; [Br-] ; COC(=O)C=C1CCC(C(C)(C)C)CC1]

NaH (1.56 g, 64.8 mmol, 60% in mineral oil) was added to a solution of (2-methoxy-2-oxoethyl)triphenylphosphonium (7.99 g, 35.6 mmol) in THF (50 mL) at 0° C. under nitrogen atmosphere. After 20 minutes, 4-tert-butylcyclohexanone (5.0 g, 32.4 mmol) was added dropwise. The reaction was warmed to room temperature and stirred for 3 hours. Upon completion, the reaction was quenched with water (20 mL) at 0° C. The resulting mixture was extracted with EtOAc (4×10 mL). The combined organic layers were dried over MgSO₄, filtered and concentrated under reduced pressure. The crude residue was purified by silica gel column chromatography, eluted with PE/EA (7:1) to afford methyl 2-(4-(tert-butyl)cyclohexylidene)acetate (6.0 g, 88% yield) as a colorless oil. LCMS (ESI) [M+H]⁺: 211.2

### Step 2: methyl 2-(4-(tert-butyl)cyclohexyl)acetate

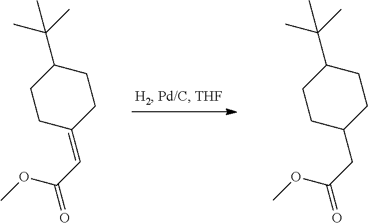

A solution of methyl 2-(4-(tert-butyl)cyclohexylidene)acetate (6.0 g, 26.7 mmol) and Pd/C (2.85 g, 26.7 mmol, 10 wt % Pd on carbon) in THF (50 mL) was stirred at room temperature overnight under hydrogen atmosphere. Upon completion, the mixture was filtered over celite and washed with THF (4×10 mL). The filtrate was concentrated under reduced pressure. The crude residue was purified by silica gel column chromatography, eluted with DCM/MeOH (10:1) to afford methyl 2-(4-(tert-butyl)cyclohexyl)acetate (4.0 g, 66% yield) as a colorless oil. LCMS (ESI) [M+H]⁺: 213.5

### Step 3: 2-(4-(tert-butyl)cyclohexyl)acetic acid

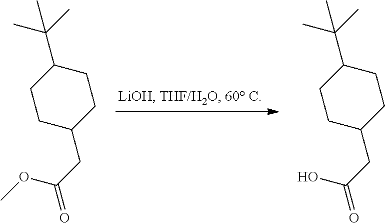

A mixture of methyl 2-(4-(tert-butyl)cyclohexyl)acetate (4.0 g, 9.59 mmol) and LiOH monohydrate (5.58 g, 23.2 mmol) in THF (40 mL) and H₂O (10 mL) was stirred at 60° C. overnight. Upon completion, the mixture was poured into water (50 mL) and acidified to pH=1 with conc. HCl. The aqueous layer was extracted with EtOAc (2×100 mL). The combined organic layers were concentrated under reduced pressure to give 2-(4-(tert-butyl)cyclohexyl)acetic acid (3.4 g, 91% yield) as a white solid, which was used in the next step without further purification.

### Step 4: 2-(6-bromo-1H-inden-3-yl)-2-(4-(tert-butyl)cyclohexyl)acetic acid

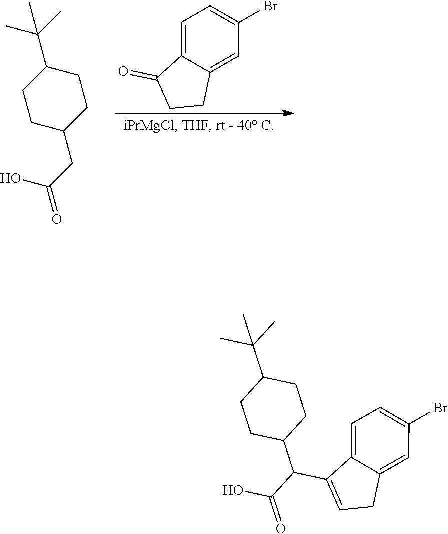

A solution of 2-(4-(tert-butyl)cyclohexyl)acetic acid (3.4 g, 19.3 mmol) in THF (30 mL) was treated with isopropylmagnesium chloride (14 mL, 28 mmol, 2.0 M in THF) at room temperature under nitrogen atmosphere. After 1 hour, 5-bromo-2,3-dihydroinden-1-one (4.05 g, 19.3 mmol) was added dropwise. The resulting mixture was heated at 40° C. for 1 hour. Upon completion, the reaction was cooled to 0° C. and quenched by the slow addition of water (50 mL). The mixture was acidified to pH=1 with conc. HCl acid. The aqueous layer was extracted with EtOAc (5×100 mL). The combined organic layers were dried over MgSO₄, filtered and concentrated under reduced pressure. The crude residue was purified by reversed-phase flash chromatography (conditions: column, C18 silica gel; mobile phase A: Water (0.1% FA), Mobile Phase B: MeCN; Gradient (B %): 60% to 70% in 10 min; detector, UV 254 nm) to yield 2-(6-bromo-1H-inden-3-yl)-2-(4-(tert-butyl)cyclohexyl)acetic acid (2.8 g, 37% yield) an off-white solid. LCMS (ESI) [M+H]⁺: 391.2/393.2

### Step 5: 2-(6-bromo-1H-inden-3-yl)-2-(4-(tert-butyl)cyclohexyl)-N-(2′,4′,6′-trimethyl-[1,1′-biphenyl]-4-yl)acetamide

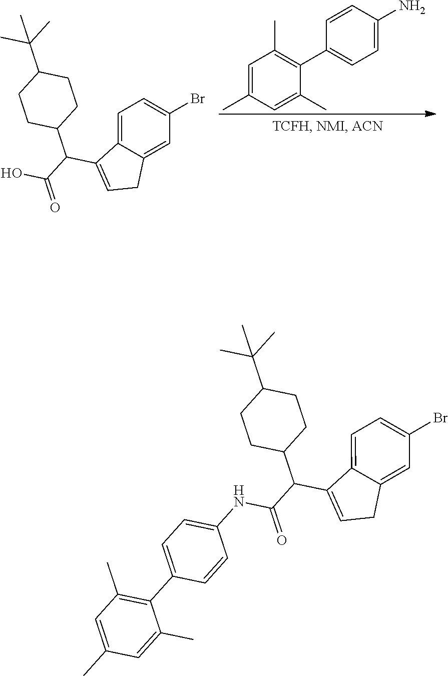

A mixture of 2-(6-bromo-1H-inden-3-yl)-2-(4-(tert-butyl)cyclohexyl)acetic acid (2.8 g, 7.1 mmol), 2′,4′,6′-trimethyl-[1,1′-biphenyl]-4-amine (1.51 g, 7.1 mmol), TCFH (3.0 g, 10.7 mmol) and 1-methyl-1H-imidazole (1.76 g, 21.4 mmol) in acetonitrile (23 mL) was stirred at room temperature for 2 hours. Upon completion, the resulting mixture was concentrated under reduced pressure. The crude residue was purified by silica gel column chromatography, eluted with PE/EA (5:1) to afford 2-(6-bromo-1H-inden-3-yl)-2-(4-(tert-butyl)cyclohexyl)-N-(2′,4′,6′-trimethyl-[1,1′-biphenyl]-4-yl)acetamide (3.3 g, 79% yield) as a white solid. LCMS (ESI) [M+H]⁺: 584.2/586.2

### Step 6: 3-(1-(4-(tert-butyl)cyclohexyl)-2-oxo-2-((2′,4′,6′-trimethyl-[1,1′-biphenyl]-4-yl)amino)ethyl)-1H-indene-6-carboxylic acid

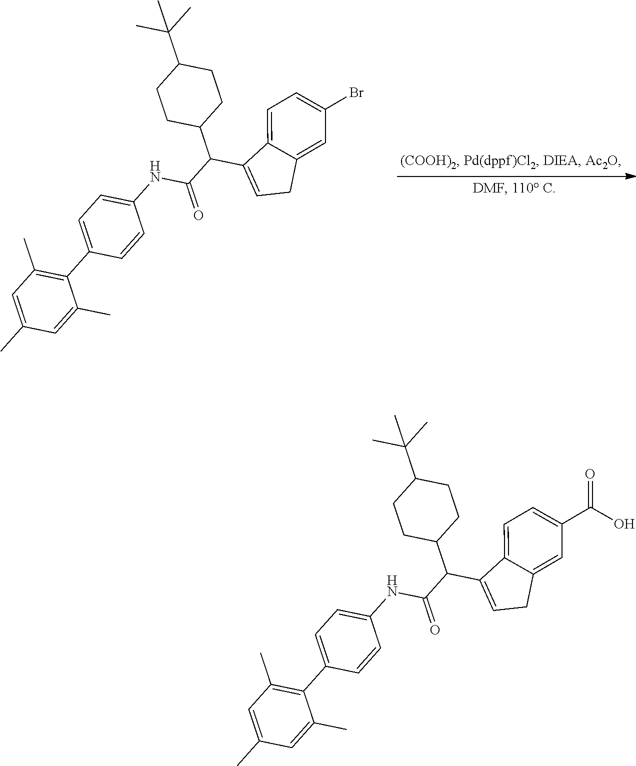 [SMILES: Cc1cc(C)c(-c2ccc(NC(=O)C(C3=CCc4cc(Br)ccc43)C3CCC(C(C)(C)C)CC3)cc2)c(C)c1 ; Cc1cc(C)c(-c2ccc(NC(=O)C(C3=CCc4cc(C(=O)O)ccc43)C3CCC(C(C)(C)C)CC3)cc2)c(C)c1]

A mixture of 2-(6-bromo-1H-inden-3-yl)-2-(4-(tert-butyl)cyclohexyl)-N-(2′,4′,6′-trimethyl-[1,1′-biphenyl]-4-yl)acetamide (3.3 g, 5.6 mmol), oxalic acid anhydrous (1.02 g, 11.8 mmol), Pd(OAc)₂ (0.13 g, 0.54 mmol) and DIEA (2.19 g, 16.2 mmol) in DMF (40 mL) was heated at 110° C. for 3 hours under nitrogen atmosphere. Upon completion, the resulting solution was concentrated under reduced pressure. The crude residue was purified by reversed-phase flash chromatography (conditions: column, C18 silica gel; mobile phase A: Water (0.1% FA), Mobile Phase B: MeCN; Gradient (B %): 60% to 80% in 10 min; detector, UV 254 nm) to give 3-(1-(4-(tert-butyl)cyclohexyl)-2-oxo-2-((2′,4′,6′-trimethyl-[1,1′-biphenyl]-4-yl)amino)ethyl)-1H-indene-6-carboxylic acid (2.9 g, 94% yield) as an off-white solid. LCMS (ESI) [M+H]⁺: 550.3

### Step 7: 3-((S)-1-((1r,4S)-4-(tert-butyl)cyclohexyl)-2-oxo-2-((2′,4′,6′-trimethyl-[1,1′-biphenyl]-4-yl)amino)ethyl)-1H-indene-6-carboxylic acid, 3-((R)-1-((1r,4R)-4-(tert-butyl)cyclohexyl)-2-oxo-2-((2′,4′,6′-trimethyl-[1,1′-biphenyl]-4-yl)amino)ethyl)-1H-indene-6-carboxylic acid, 3-((S)-1-((1s,4R)-4-(tert-butyl)cyclohexyl)-2-oxo-2-((2′,4′,6′-trimethyl-[1,1′-biphenyl]-4-yl)amino)ethyl)-1H-indene-6-carboxylic acid and 3-((R)-1-((1s,4S)-4-(tert-butyl)cyclohexyl)-2-oxo-2-((2′,4′,6′-trimethyl-[1,1′-biphenyl]-4-yl)amino)ethyl)-1H-indene-6-carboxylic acid

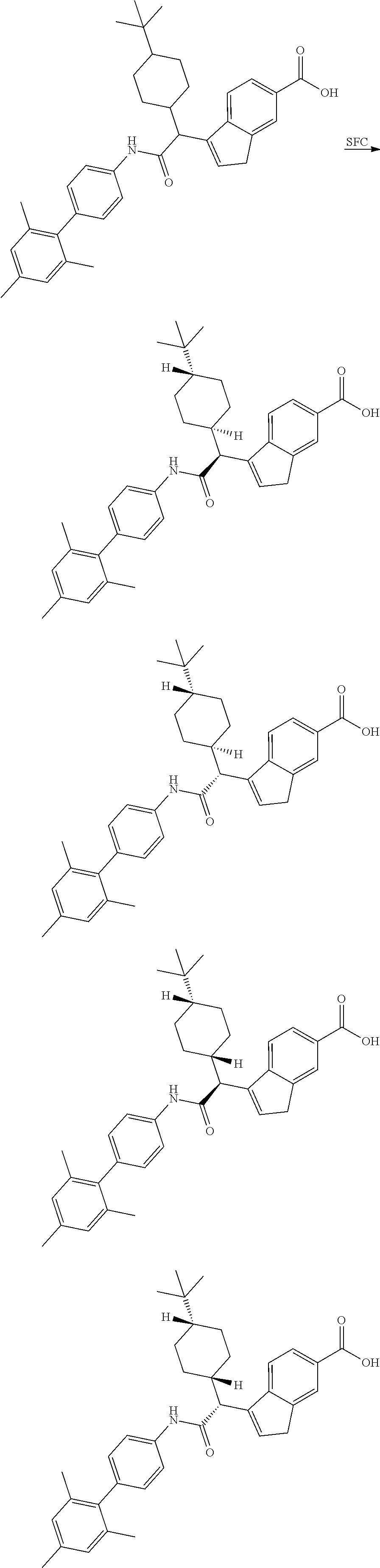 [SMILES: Cc1cc(C)c(-c2ccc(NC(=O)C(C3=CCc4cc(C(=O)O)ccc43)C3CCC(C(C)(C)C)CC3)cc2)c(C)c1 ; "SFC" ; Cc1cc(C)c(-c2ccc(NC(=O)[C@H](C3=CCc4cc(C(=O)O)ccc43)[C@H]3CC[C@H](C(C)(C)C)CC3)cc2)c(C)c1 ; Cc1cc(C)c(-c2ccc(NC(=O)[C@@H](C3=CCc4cc(C(=O)O)ccc43)[C@H]3CC[C@H](C(C)(C)C)CC3)cc2)c(C)c1 ; Cc1cc(C)c(-c2ccc(NC(=O)[C@H](C3=CCc4cc(C(=O)O)ccc43)[C@H]3CC[C@@H](C(C)(C)C)CC3)cc2)c(C)c1 ; Cc1cc(C)c(-c2ccc(NC(=O)[C@@H](C3=CCc4cc(C(=O)O)ccc43)[C@H]3CC[C@@H](C(C)(C)C)CC3)cc2)c(C)c1]

3-(1-(4-(tert-butyl)cyclohexyl)-2-oxo-2-((2′,4′,6′-trimethyl-[1,1′-biphenyl]-4-yl)amino)ethyl)-1H-indene-6-carboxylic acid (3.4 g) was separated by prep chiral SFC (conditions: Column: CHIRAL ART Cellulose-SB, 3*25 cm, 5 m; Mobile Phase A: CO₂, Mobile Phase B: MeOH (20 mM NH₃); Flow rate: 90 mL/min; Gradient (B %): isocratic 35% B; Column Temperature (° C.): 35; Back Pressure (bar): 100; Wave Length: 222 nm; RT1 (min): 5.7; RT2 (min): 6.7; RT3 (min): 12.68; RT4 (min): 15.67) to afford 3-((S)-1-((1r,4S)-4-(tert-butyl)cyclohexyl)-2-oxo-2-((2′,4′,6′-trimethyl-[1,1′-biphenyl]-4-yl)amino)ethyl)-1H-indene-6-carboxylic acid (450 mg, 13% yield, 1^{st} fraction, RT1 (min): 5.7), 3-((R)-1-((1r,4R)-4-(tert-butyl)cyclohexyl)-2-oxo-2-((2′,4′,6′-trimethyl-[1,1′-biphenyl]-4-yl)amino)ethyl)-1H-indene-6-carboxylic acid (450 mg, 13% yield, 2^{nd} fraction, RT2 (min): 6.7), 3-((S)-1-((1s,4R)-4-(tert-butyl)cyclohexyl)-2-oxo-2-((2′,4′,6′-trimethyl-[1,1′-biphenyl]-4-yl)amino)ethyl)-1H-indene-6-carboxylic acid (1.0 g, 29% yield, 3^{rd} fraction, RT3 (min): 12.68) and 3-((R)-1-((1s,4S)-4-(tert-butyl)cyclohexyl)-2-oxo-2-((2′,4′,6′-trimethyl-[1,1′-biphenyl]-4-yl)amino)ethyl)-1H-indene-6-carboxylic acid (1.0 g, 29% yield, 4^{th} fraction, RT4 (min): 15.67) as a yellow solid. LCMS (ESI) [M+H]⁺: 550.3

### Step 8: 1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid, 1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid, 1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid and 1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid

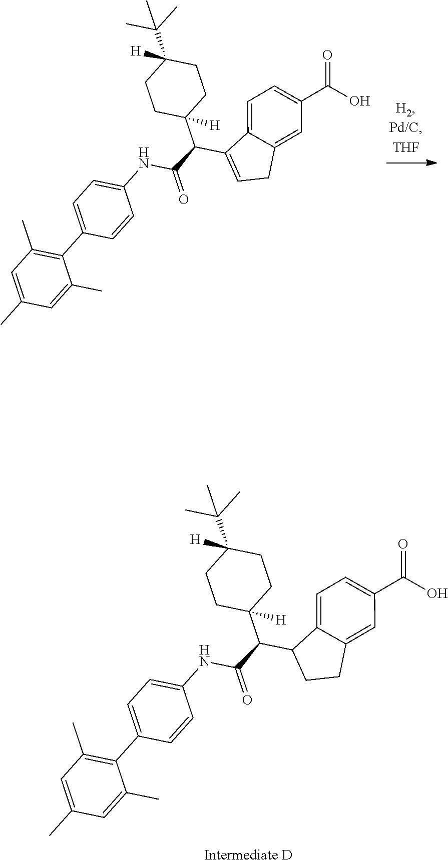 [SMILES: Cc1cc(C)c(-c2ccc(NC(=O)[C@H](C3=CCc4cc(C(=O)O)ccc43)[C@H]3CC[C@H](C(C)(C)C)CC3)cc2)c(C)c1 ; Cc1cc(C)c(-c2ccc(NC(=O)[C@H](C3CCc4cc(C(=O)O)ccc43)[C@H]3CC[C@H](C(C)(C)C)CC3)cc2)c(C)c1]

A mixture of 3-((S)-1-((1r,4S)-4-(tert-butyl)cyclohexyl)-2-oxo-2-((2′,4′,6′-trimethyl-[1,1′-biphenyl]-4-yl)amino)ethyl)-1H-indene-6-carboxylic acid (400 mg, 0.72 mmol) and Pd/C (77.4 mg, 0.73 mmol, 10 wt % Pd on carbon) in THF (4 mL) was stirred at room temperature for 5 hours under hydrogen atmosphere. Upon completion, the mixture was filtered over celite. The filter cake was washed with DCM (5×10 mL). The filtrate was concentrated under reduced pressure to afford 1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate D) (300 mg, 75% yield) as a white solid, which was used in the next step without further purification. LCMS (ESI) [M+H]⁺: 552.3

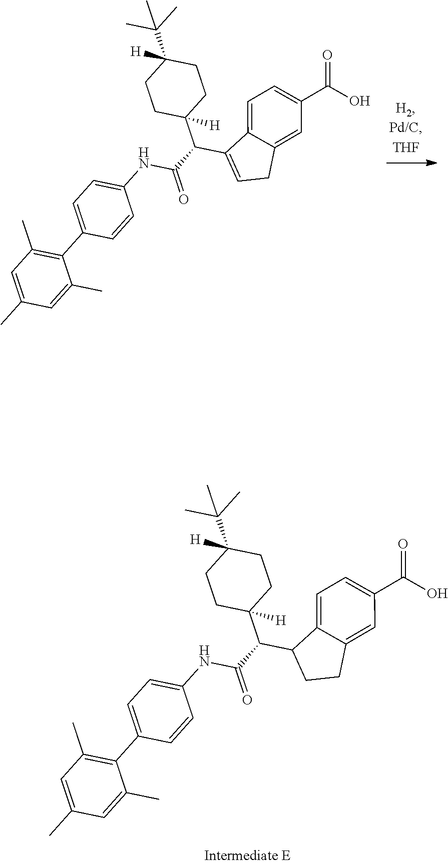 [SMILES: Cc1cc(C)c(-c2ccc(NC(=O)[C@@H](C3=CCc4cc(C(=O)O)ccc43)[C@H]3CC[C@H](C(C)(C)C)CC3)cc2)c(C)c1 ; Cc1cc(C)c(-c2ccc(NC(=O)[C@@H](C3CCc4cc(C(=O)O)ccc43)[C@H]3CC[C@H](C(C)(C)C)CC3)cc2)c(C)c1]

A mixture of 3-((R)-1-((1r,4R)-4-(tert-butyl)cyclohexyl)-2-oxo-2-((2′,4′,6′-trimethyl-[1,1′-biphenyl]-4-yl)amino)ethyl)-1H-indene-6-carboxylic acid (400 mg, 0.72 mmol) and Pd/C (77.4 mg, 0.73 mmol, 10 wt % Pd on carbon) in THF (4 mL) was stirred at room temperature for 5 hours under hydrogen atmosphere. Upon completion, the mixture was filtered over celite. The filter cake was washed with DCM (5×10 mL). The filtrate was concentrated under reduced pressure to afford 1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate E) (312 mg, 75% yield) as a white solid, which was used in the next step without further purification. LCMS (ESI) [M+H]⁺: 552.3

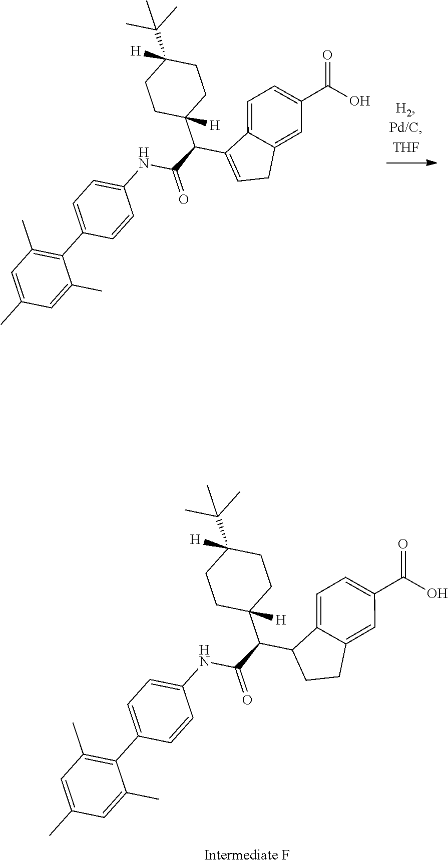 [SMILES: Cc1cc(C)c(-c2ccc(NC(=O)[C@H](C3=CCc4cc(C(=O)O)ccc43)[C@H]3CC[C@@H](C(C)(C)C)CC3)cc2)c(C)c1 ; Cc1cc(C)c(-c2ccc(NC(=O)[C@H](C3CCc4cc(C(=O)O)ccc43)[C@H]3CC[C@@H](C(C)(C)C)CC3)cc2)c(C)c1]

A solution of 3-((S)-1-((1s,4R)-4-(tert-butyl)cyclohexyl)-2-oxo-2-((2′,4′,6′-trimethyl-[1,1′-biphenyl]-4-yl)amino)ethyl)-1H-indene-6-carboxylic acid (600 mg, 1.0 mmol) and Pd/C (11.6 mg, 0.109 mmol, 10 wt % Pd on carbon) in THF (6 mL) was stirred at room temperature for 2 hours under hydrogen atmosphere. Upon completion, the resulting mixture was filtered over celite. The filter cake was washed with DCM (3×5 mL). The filtrate was concentrated under reduced pressure to afford 1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate F) (500 mg, 65% yield) as a white solid, which was used in the next step without further purification. LCMS ESI [M+H]⁺: 552.3

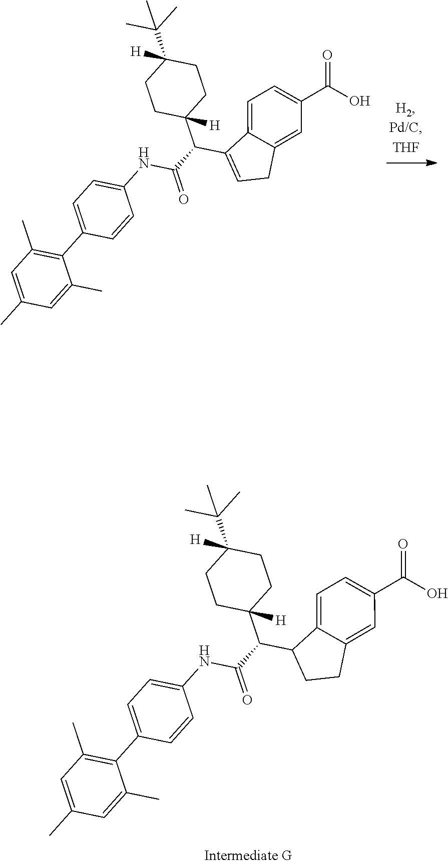 [SMILES: Cc1cc(C)c(-c2ccc(NC(=O)[C@@H](C3=CCc4cc(C(=O)O)ccc43)[C@H]3CC[C@@H](C(C)(C)C)CC3)cc2)c(C)c1 ; Cc1cc(C)c(-c2ccc(NC(=O)[C@@H](C3CCc4cc(C(=O)O)ccc43)[C@H]3CC[C@@H](C(C)(C)C)CC3)cc2)c(C)c1]

A mixture of 3-((R)-1-((1s,4S)-4-(tert-butyl)cyclohexyl)-2-oxo-2-((2′,4′,6′-trimethyl-[1,1′-biphenyl]-4-yl)amino)ethyl)-1H-indene-6-carboxylic acid (600 mg, 1.0 mmol) and Pd/C (11.6 mg, 0.109 mmol, 10 wt % Pd on carbon) in THF (6 mL) was stirred at room temperature for 2 hours under hydrogen atmosphere. Upon completion, the resulting mixture was filtered over celite. The filter cake was washed with DCM (3×5 mL). The filtrate was concentrated under reduced pressure to afford 1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate G) (335 mg, 54% yield) as a white solid, which was used in the next step without further purification. LCMS (ESI) [M+H]⁺: 552.3

### Example 1: 3-(1-(1-((1s,4s)-4-(tert-butyl)cyclohexyl)-3-(4-((trifluoromethyl)thio)phenyl)ureido)-2,3-dihydro-1H-indene-5-carboxamido)propanoic acid

### Step 1: methyl 3-(1-(1-((1r,4r)-4-(tert-butyl)cyclohexyl)-3-(4-((trifluoromethyl)thio)phenyl)ureido)-2,3-dihydro-1H-indene-5-carboxamido)propanoate

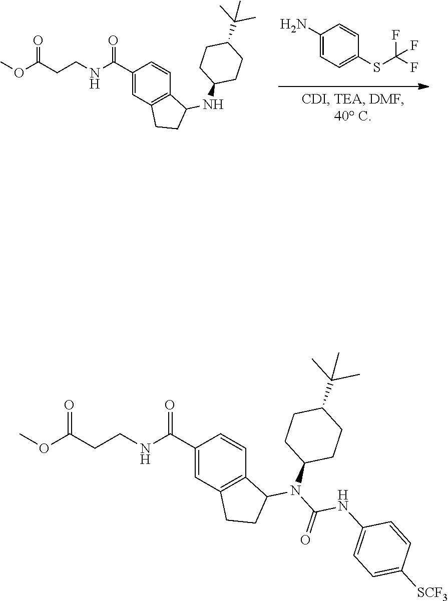

A solution of methyl 3-(1-(((1r,4r)-4-(tert-butyl)cyclohexyl)amino)-2,3-dihydro-1H-indene-5-carboxamido)propanoate (Intermediate B; 60 mg, 0.150 mmol), 4-((trifluoromethyl)thio)aniline (87 mg, 0.45 mmol), CDI (73 mg, 0.45 mmol) and triethylamine (45 mg, 0.45 mmol) in DMF (1 mL) was stirred at 40° C. for 2 hours. Upon completion, the resulting mixture was poured into water (5 mL). The aqueous layer was extracted with EA (2×5 mL). The combined organic layers were washed with H₂O (1×10 mL), dried over anhydrous Na₂SO₄, filtered and concentrated under reduced pressure. The crude residue was purified by silica gel column chromatography, eluted with PE/EA (0-40%) to afford methyl 3-(1-(1-((1r,4r)-4-(tert-butyl)cyclohexyl)-3-(4-((trifluoromethyl)thio)phenyl)ureido)-2,3-dihydro-1H-indene-5-carboxamido)propanoate (50 mg, crude) as a yellow oil. LCMS (ESI) [M+H]⁺: 620.

### Step 2: 3-(1-(1-((1r,4r)-4-(tert-butyl)cyclohexyl)-3-(4-((trifluoromethyl)thio)phenyl)ureido)-2,3-dihydro-1H-indene-5-carboxamido)propanoic acid

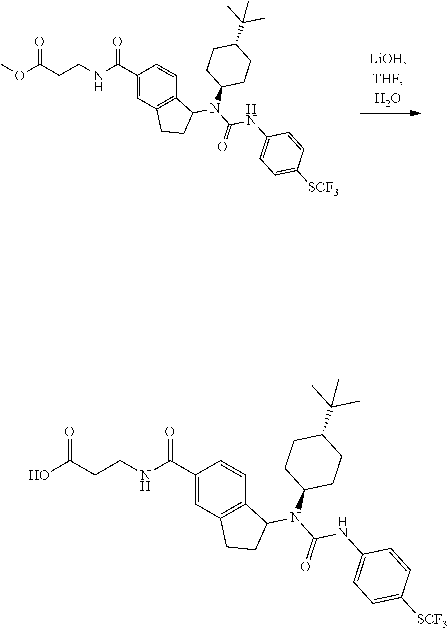

A solution of methyl 3-(1-(1-((1r,4r)-4-(tert-butyl)cyclohexyl)-3-(4-((trifluoromethyl)thio)phenyl)ureido)-2,3-dihydro-1H-indene-5-carboxamido)propanoate (50 mg, 0.081 mmol) and LiOH monohydrate (3.9 mg, 0.16 mmol) in H₂O (1 mL) and THF (1 mL) was stirred at 25° C. for 1 hour. Upon completion, the mixture was poured into water (10 mL), acidified to pH=2 with HCl (1 M in water) and extracted with EA (3×50 mL). The combined organic layers were washed with H₂O (1×10 mL), dried over anhydrous Na₂SO₄, filtered and concentrated under reduced pressure. The crude residue was purified by reversed-phase flash chromatography (conditions: column, C18 silica gel; mobile phase, ACN in H₂O, 10% to 50% gradient in 10 min; detector, UV 254 nm) to give 3-(1-(1-((1r,4r)-4-(tert-butyl)cyclohexyl)-3-(4-((trifluoromethyl)thio)phenyl)ureido)-2,3-dihydro-1H-indene-5-carboxamido)propanoic acid (30 mg, 61.4% yield) as a white solid. LCMS (ESI) [M+H]⁺: 606.

### Step 3: 3-((S)-1-(1-((1r,4S)-4-(tert-butyl)cyclohexyl)-3-(4-((trifluoromethyl)thio)phenyl)ureido)-2,3-dihydro-1H-indene-5-carboxamido)propanoic acid and 3-((R)-1-(1-((1r,4R)-4-(tert-butyl)cyclohexyl)-3-(4-((trifluoromethyl)thio)phenyl)ureido)-2,3-dihydro-1H-indene-5-carboxamido)propanoic acid

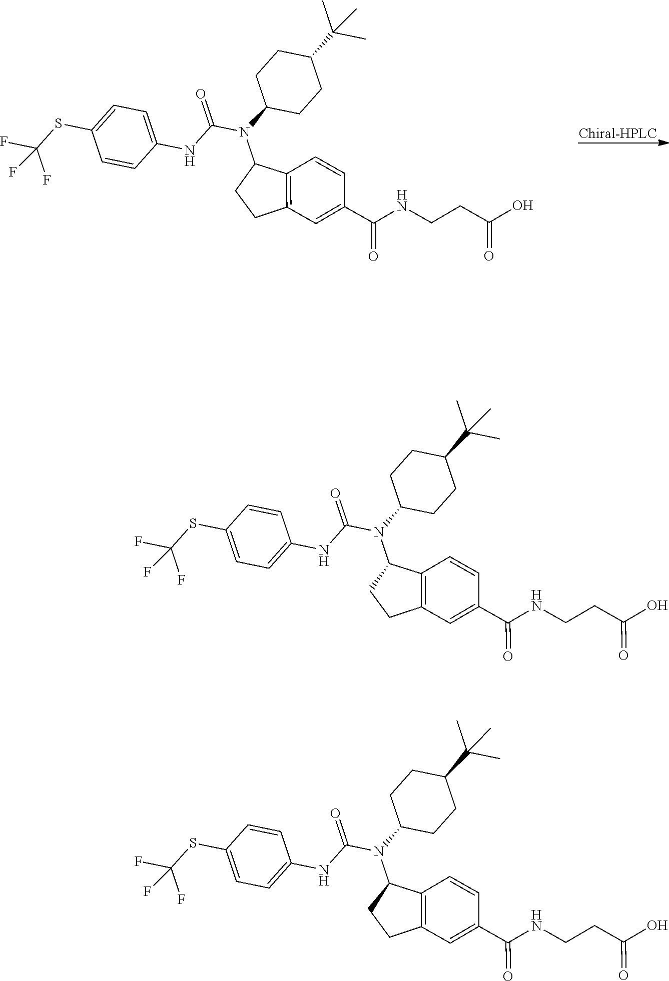 [SMILES: CC(C)(C)[C@H]1CC[C@H](N(C(=O)Nc2ccc(SC(F)(F)F)cc2)C2CCc3cc(C(=O)NCCC(=O)O)ccc32)CC1 ; CC(C)(C)[C@H]1CC[C@H](N(C(=O)Nc2ccc(SC(F)(F)F)cc2)[C@H]2CCc3cc(C(=O)NCCC(=O)O)ccc32)CC1 ; CC(C)(C)[C@H]1CC[C@H](N(C(=O)Nc2ccc(SC(F)(F)F)cc2)[C@@H]2CCc3cc(C(=O)NCCC(=O)O)ccc32)CC1]

3-(1-(1-((1r,4r)-4-(tert-butyl)cyclohexyl)-3-(4-((trifluoromethyl)thio)phenyl)ureido)-2,3-dihydro-1H-indene-5-carboxamido)propanoic acid (30 mg, 0.050 mmol) was separated by prep-Chiral-HPLC (Column: CHIRAL ART Cellulose-SB, 3*25 cm, 5 μm; Mobile Phase A: MtBE:Hex=1:1 (0.1% FA), Mobile Phase B: MeOH; Flow rate: 40 mL/min; Gradient: isocratic 10% B; Wave Length: 202/262 nm; RT1 (min): 6.1; RT2 (min): 9.4) to afford first peak 3-((S)-1-(1-((1r,4S)-4-(tert-butyl)cyclohexyl)-3-(4-((trifluoromethyl)thio)phenyl)ureido)-2,3-dihydro-1H-indene-5-carboxamido)propanoic acid (Compound 1a) (Method A, Peak 1, 0.73 min; 8.9 mg, 29.7% yield) and second peak 3-((R)-1-(1-((1r,4R)-4-(tert-butyl)cyclohexyl)-3-(4-((trifluoromethyl)thio)phenyl)ureido)-2,3-dihydro-1H-indene-5-carboxamido)propanoic acid (Compound 1b) (Method A, Peak 2, 1.07 min; 8.4 mg, 28.0% yield).

| Compound# | Characterization Data |
|---|---|
| 1a | LCMS (ESI) [M + H]⁺: 606.3 |
| | ¹H NMR (400 MHz, Methanol-d4) δ 7.71 (s, 1H), 7.66 (d, J = 7.9 Hz, 1H), 7.47 (d, J = 8.8 Hz, 2H), |
| | 7.26 (d, J = 7.8 Hz, 1H), 7.22 (d, J = 8.6 Hz, 2H), 5.16 (dd, J = 8.6 Hz, 1H), 3.87 (bs, 1H), 3.61 (dd, |
| | J = 6.9 Hz, 2H), 3.21-3.11 (m, 1H), 2.97 (ddd, J = 16.8, 8.9 Hz, 1H), 2.60 (dd, J = 6.9 Hz, 2H), |
| | 2.57-2.44 (m, 1H), 2.44-2.34 (m, 1H), 2.05-2.00 (m, 1H), 1.92 (dd, J = 13.1 Hz, 3H), 1.82 (dd, |
| | J = 11.8 Hz, 2H), 1.31-1.20 (m, 2H), 1.13-1.04 (m, 1H), 0.89 (s, 9H). |
| 1b | LCMS (ESI) [M + H]⁺: 606.3 |
| | ¹H NMR (400 MHz, Methanol-d4) δ 7.70 (s, 1H), 7.66 (d, J = 7.9 Hz, 1H), 7.47 (d, J = 8.8 Hz, 2H), |
| | 7.26 (d, J = 7.9 Hz, 1H), 7.22 (d, J = 8.8 Hz, 2H), 5.16 (dd, J = 8.6 Hz, 1H), 3.87 (bs, 1H), 3.61 (dd, |
| | J = 6.9 Hz, 2H), 3.22-3.10 (m, 1H), 2.97 (ddd, J = 16.7, 8.8 Hz, 1H), 2.61 (dd, J = 6.9 Hz, 2H), |
| | 2.54-2.44 (m, 1H), 2.43-2.34 (m, 1H), 2.03 (d, J = 11.0 Hz, 1H), 1.92 (dd, J = 12.4 Hz, 3H), |
| | 1.82 (dd, J = 11.1 Hz, 2H), 1.31-1.20 (m, 2H), 1.12-1.04 (m, 1H), 0.89 (s, 9H). |

### Example 2: 3-(1-(1-(4-cyclohexylphenyl)-3-(4-((trifluoromethyl)thio)phenyl)ureido)-2,3-dihydro-1H-indene-5-carboxamido)propanoic acid

### Step 1: methyl 3-(1-(1-(4-cyclohexylphenyl)-3-(4-((trifluoromethyl)thio)phenyl)ureido)-2,3-dihydro-1H-indene-5-carboxamido)propanoate

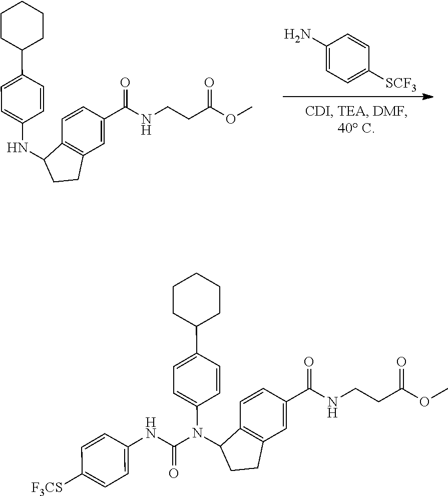

A solution of methyl 3-(1-((4-cyclohexylphenyl)amino)-2,3-dihydro-1H-indene-5-carboxamido)propanoate (Intermediate C; 70 mg, 0.17 mmol), 4-((trifluoromethyl)thio)aniline (161 mg, 0.83 mmol), CDI (55 mg, 0.34 mmol) and triethylamine (84 mg, 0.83 mmol) in DMF (1 mL) was stirred at 40° C. for 1 hour. Upon completion, the resulting mixture was extracted with EA (3×100 mL). The combined organic layers were washed with H₂O (1×50 mL), dried over anhydrous Na₂SO₄, filtered and concentrated under reduced pressure. The crude residue was purified by silica gel column chromatography, eluted with PE/EA (0-50%) to afford methyl 3-(1-(1-(4-cyclohexylphenyl)-3-(4-((trifluoromethyl)thio)phenyl)ureido)-2,3-dihydro-1H-indene-5-carboxamido)propanoate (50 mg, crude) as a white oil. LCMS (ESI) [M+H]⁺: 640.

### Step 2: 3-(1-(1-(4-cyclohexylphenyl)-3-(4-((trifluoromethyl)thio)phenyl)ureido)-2,3-dihydro-1H-indene-5-carboxamido)propanoic acid

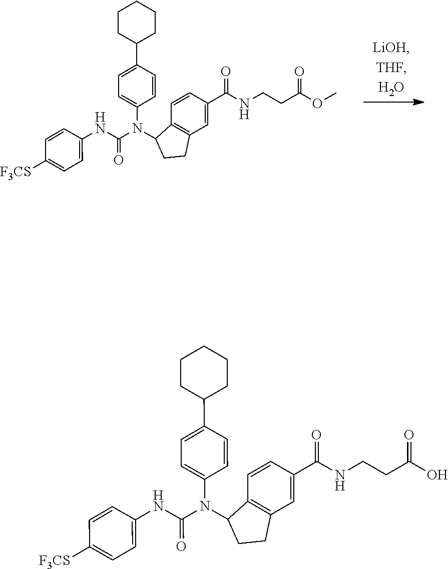

A solution of methyl 3-(1-(1-(4-cyclohexylphenyl)-3-(4-((trifluoromethyl)thio)phenyl)ureido)-2,3-dihydro-1H-indene-5-carboxamido)propanoate (50 mg, 0.078 mmol) and LiOH monohydrate (3.7 mg, 0.16 mmol) in H₂O (1 mL) THF (1 mL) was stirred at 25° C. for 1 hour. Upon completion, the mixture was poured into water (50 mL), acidified to pH=2 with HCl and then extracted with EA (3×50 mL). The combined organic layers were washed with H₂O (1×20 mL), dried over anhydrous Na₂SO₄, filtered and concentrated under reduced pressure. The crude residue was purified by reversed-phase flash chromatography (conditions: column, C18 silica gel; mobile phase, MeCN in H₂O, 10% to 50% gradient in 10 min; detector, UV 254 nm) to afford 3-(1-(1-(4-cyclohexylphenyl)-3-(4-((trifluoromethyl)thio)phenyl)ureido)-2,3-dihydro-1H-indene-5-carboxamido)propanoic acid (30 mg) as a white solid. LCMS (ESI) [M+H]⁺: 626.

### Step 3: (R)-3-(1-(1-(4-cyclohexylphenyl)-3-(4-((trifluoromethyl)thio)phenyl)ureido)-2,3-dihydro-1H-indene-5-carboxamido)propanoic acid and (S)-3-(1-(1-(4-cyclohexylphenyl)-3-(4-((trifluoromethyl)thio)phenyl)ureido)-2,3-dihydro-1H-indene-5-carboxamido)propanoic acid

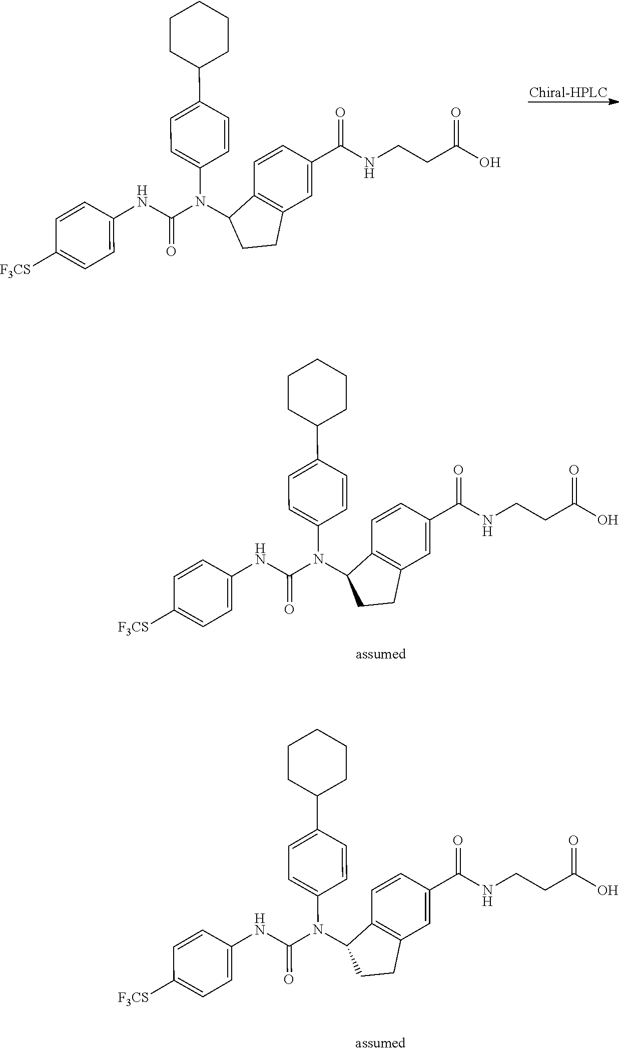

3-(1-(1-(4-cyclohexylphenyl)-3-(4-((trifluoromethyl)thio)phenyl)ureido)-2,3-dihydro-1H-indene-5-carboxamido)propanoic acid (30 mg, 0.048 mmol) was separated by prep-Chiral-HPLC (Column: Chiral NX(2) 5 μm, 250*30 mm; Mobile Phase A: Hex (0.1% FA), Mobile Phase B: EtOH; Flow rate: 40 mL/min; Gradient: isocratic 30% B; Wave Length: 204/262 nm; RT1 (min): 8.12; RT2 (min): 9.57) to afford first peak (R)-3-(1-(1-(4-cyclohexylphenyl)-3-(4-((trifluoromethyl)thio)phenyl)ureido)-2,3-dihydro-1H-indene-5-carboxamido)propanoic acid (Compound 2a) (Method B, Peak 1, 1.06 min; 11.5 mg, 38.3% yield) and second peak (S)-3-(1-(1-(4-cyclohexylphenyl)-3-(4-((trifluoromethyl)thio)phenyl)ureido)-2,3-dihydro-1H-indene-5-carboxamido)propanoic acid (Compound 2b) (Method A, Peak 2, 1.33 min; 6.8 mg, 22.7% yield).

| Compound# | Characterization Data |
|---|---|
| 2a | LCMS (ESI) [M − H]⁻: 624.2 |
| | ¹H NMR (400 MHz, Methanol-d₄) δ 7.70 (dd, J = 7.9, 1.8 Hz, 1H), 7.57 (d, J = 8.0 Hz, 1H), |
| | 7.53 (d, J = 8.6 Hz, 3H), 7.45 (d, J = 8.9 Hz, 2H), 7.20 (d, J = 7.3 Hz, 2H), 6.96 (d, J = 8.3 Hz, |
| | 2H), 6.22 (dd, J = 7.9, 6.2 Hz, 1H), 3.61 (dd, J = 6.9 Hz, 2H), 2.77-2.66 (m, 1H), 2.62 (dd, J = |
| | 6.9 Hz, 2H), 2.55-2.48 (m, 1H), 2.46-2.32 (m, 2H), 2.13-1.95 (m, 1H), 1.83 (d, J = 7.4 |
| | Hz, 4H), 1.74 (d, J = 10.9 Hz, 1H), 1.49-1.35 (m, 4H), 1.34-1.26 (m, 1H). |
| 2b | LCMS (ESI) [M − H]⁻: 624.2 |
| | ¹H NMR (400 MHz, Methanol-d₄) δ 7.70 (dd, J = 7.9, 1.8 Hz, 1H), 7.57 (d, J = 8.0 Hz, 1H), |
| | 7.53 (d, J = 8.8 Hz, 3H), 7.45 (d, J = 8.9 Hz, 2H), 7.20 (d, J = 8.8 Hz, 2H), 6.95 (d, J = 8.3 Hz, |
| | 2H), 6.28-6.19 (m, 1H), 3.61 (dd, J = 6.9 Hz, 2H), 2.78-2.67 (m, 1H), 2.62 (dd, J = 6.9 Hz, |
| | 2H), 2.55-2.49 (m, 1H), 2.48-2.36 (m, 2H), 2.10-1.95 (m, 1H), 1.89-1.79 (m, 4H), 1.74 |
| | (d, J = 11.4 Hz, 1H), 1.46-1.37 (m, 4H), 1.34-1.24 (m, 1H). |

### Example 3: 3-(1-(N-(4-cyclohexylphenyl)-2-(4-((trifluoromethyl)thio)phenyl)acetamido)-2,3-dihydro-1H-indene-5-carboxamido)propanoic acid

### Step 1: methyl 3-(1-(N-(4-cyclohexylphenyl)-2-(4-((trifluoromethyl)thio)phenyl)acetamido)-2,3-dihydro-1H-indene-5-carboxamido)propanoate

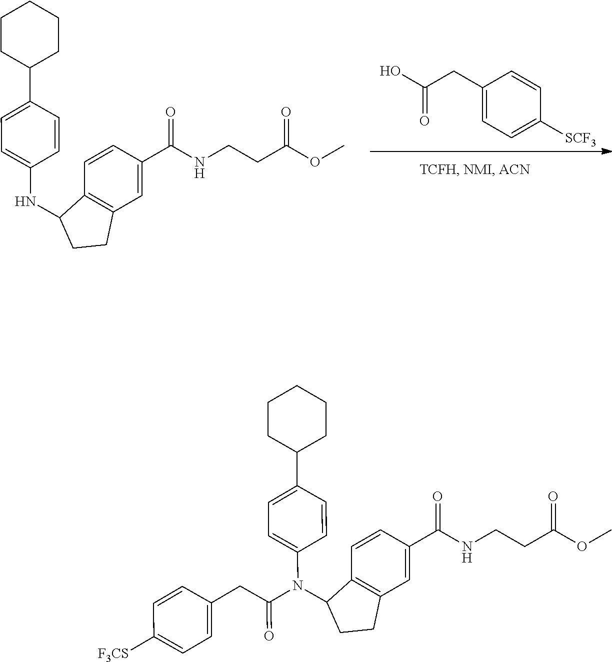

A solution of methyl 3-(1-((4-cyclohexylphenyl)amino)-2,3-dihydro-1H-indene-5-carboxamido)propanoate (Intermediate C; 70 mg, 0.17 mmol), 2-(4-((trifluoromethyl)thio)phenyl)acetic acid (197 mg, 0.83 mmol), TCFH (234 mg, 0.83 mmol) and NMI (68.3 mg, 0.83 mmol) in acetonitrile (1 mL) was stirred at 25° C. for 1 hour. Upon completion, the resulting mixture was concentrated under reduced pressure. The crude residue was purified by silica gel column chromatography, eluted with PE/EA (0-50%) to afford methyl 3-(1-(N-(4-cyclohexylphenyl)-2-(4-((trifluoromethyl)thio)phenyl)acetamido)-2,3-dihydro-1H-indene-5-carboxamido)propanoate (50 mg) as a colorless oil. LCMS (ESI) [M+H]⁺: 639.

### Step 2: 3-(1-(N-(4-cyclohexylphenyl)-2-(4-((trifluoromethyl)thio)phenyl)acetamido)-2,3-dihydro-1H-indene-5-carboxamido)propanoic acid

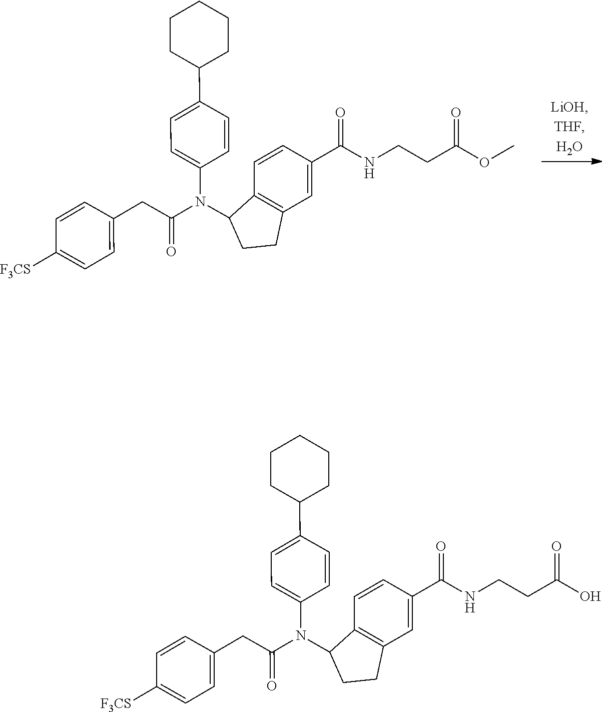

A solution of methyl 3-(1-(N-(4-cyclohexylphenyl)-2-(4-((trifluoromethyl)thio)phenyl)acetamido)-2,3-dihydro-1H-indene-5-carboxamido)propanoate (70 mg, 0.11 mmol) and LiOH monohydrate (5.3 mg, 0.22 mmol) in H₂O (1 mL) and THF (1 mL) was stirred at 25° C. for 1 hour. Upon completion, the reaction mixture was diluted with water (20 mL), acidified to pH=2 with HCl (1 M in water) and extracted with EA (3×20 mL). The combined organic layers were washed with H₂O (1×20 mL), dried over anhydrous Na₂SO₄, filtered and concentrated under reduced pressure. The crude residue was purified by reversed-phase flash chromatography (conditions: column, C18 silica gel; mobile phase, ACN in H₂O, 10% to 50% gradient in 10 min; detector, UV 254 nm) to afford 3-(1-(N-(4-cyclohexylphenyl)-2-(4-((trifluoromethyl)thio)phenyl)acetamido)-2,3-dihydro-1H-indene-5-carboxamido)propanoic acid (30 mg) as a white solid. LCMS (ESI) [M+H]⁺: 625.

### Step 3: (R)-3-(3-(N-(4-cyclohexylphenyl)-2-(4-((trifluoromethyl)thio)phenyl)acetamido)-2,3-dihydro-1H-indene-5-carboxamido)propanoic acid and (S)-3-(3-(N-(4-cyclohexylphenyl)-2-(4-((trifluoromethyl)thio)phenyl)acetamido)-2,3-dihydro-1H-indene-5-carboxamido)propanoic acid

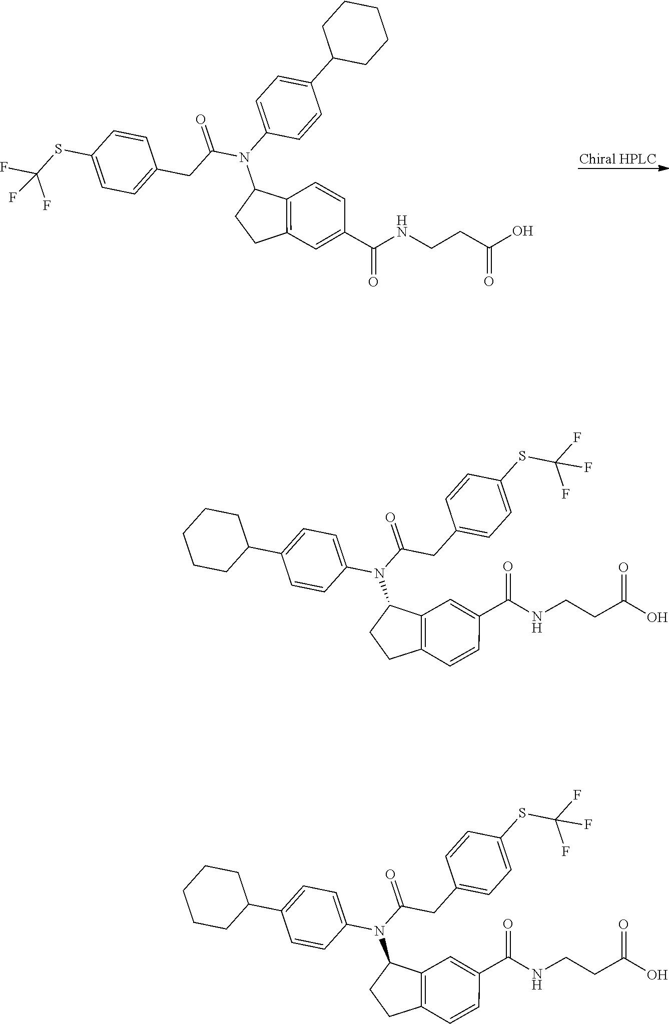 [SMILES: O=C(O)CCNC(=O)c1ccc2c(c1)CCC2N(C(=O)Cc1ccc(SC(F)(F)F)cc1)c1ccc(C2CCCCC2)cc1 ; O=C(O)CCNC(=O)c1ccc2c(c1)[C@@H](N(C(=O)Cc1ccc(SC(F)(F)F)cc1)c1ccc(C3CCCCC3)cc1)CC2 ; O=C(O)CCNC(=O)c1ccc2c(c1)[C@H](N(C(=O)Cc1ccc(SC(F)(F)F)cc1)c1ccc(C3CCCCC3)cc1)CC2]

3-(1-(N-(4-cyclohexylphenyl)-2-(4-((trifluoromethyl)thio)phenyl)acetamido)-2,3-dihydro-1H-indene-5-carboxamido)propanoic acid (30 mg, 0.048 mmol) was separated by prep-Chiral-HPLC (Column: CHIRAL ART Cellulose-SB, 3*25 cm, 5 m; Mobile Phase A: MtBE (0.1% FA), Mobile Phase B: ETOH; Flow rate: 40 mL/min; Gradient: isocratic 10% B; Wave Length: 240/204 nm; RT1 (min): 4.63; RT2 (min): 6.74) to afford first peak (R)-3-(3-(N-(4-cyclohexylphenyl)-2-(4-((trifluoromethyl)thio)phenyl)acetamido)-2,3-dihydro-1H-indene-5-carboxamido)propanoic acid (Compound 3a) (Method C, Peak 1, 0.94 min; 11.5 mg, 38.3% yield) and second peak (S)-3-(3-(N-(4-cyclohexylphenyl)-2-(4-((trifluoromethyl)thio)phenyl)acetamido)-2,3-dihydro-1H-indene-5-carboxamido)propanoic acid (Compound 3b) (Method C, Peak 2, 1.34 min; 11.1 mg, 37.0% yield).

| Compound# | Characterization Data |
|---|---|
| 3a | LCMS (ESI) [M + H]⁺: 625.3 |
| | ¹H NMR (400 MHz, Methanol-d₄) δ 7.67 (dd, J = 7.9, 1.8 Hz, 1H), 7.57-7.50 (m, 3H), 7.46 |
| | (d, J = 8.0 Hz, 1H), 7.29-7.17 (m, 1H), 7.13 (d, J = 8.3 Hz, 2H), 6.96-6.89 (m, 1H), 6.39 - |
| | 6.28 (m, 2H), 3.60 (dd, J = 6.9 Hz, 2H), 3.52 (s, 2H), 2.75-2.65 (m, 1H), 2.62 (dd, J = 6.9 |
| | Hz, 2H), 2.52-2.44 (m, 1H), 2.42-2.32 (m, 2H), 2.08-1.94 (m, 1H), 1.87-1.79 (m, 4H), |
| | 1.76-1.70 (m, 1H), 1.51-1.34 (m, 4H), 1.31-1.24 (m, 1H). |
| 3b | LCMS (ESI) [M + H]⁺: 625.3 |
| | ¹H NMR (400 MHz, Methanol-d₄) δ 7.67 (d, J = 7.9 Hz, 1H), 7.58-7.50 (m, 3H), 7.46 (d, J = |
| | 7.9 Hz, 1H), 7.29-7.18 (m, 1H), 7.13 (d, J = 8.3 Hz, 2H), 6.98-6.89 (m, 1H), 6.41-6.28 (m, |
| | 2H), 3.60 (dd, J = 6.9 Hz, 2H), 3.52 (s, 2H), 2.77-2.64 (m, 1H), 2.62 (dd, J = 6.9 Hz, 2H), 2.52- |
| | 2.46 (m, 1H), 2.44-2.32 (m, 2H), 2.08-1.95 (m, 1H), 1.87-1.77 (m, 4H), 1.76-1.70 (m, |
| | 1H), 1.45-1.34 (m, 4H), 1.33-1.23 (m, 1H). |

### Example 4: 2-(1-(1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

### Step 1: 2-(5-bromo-1-hydroxy-2,3-dihydro-1H-inden-1-yl)-2-(4-(tert-butyl)phenyl)acetic acid

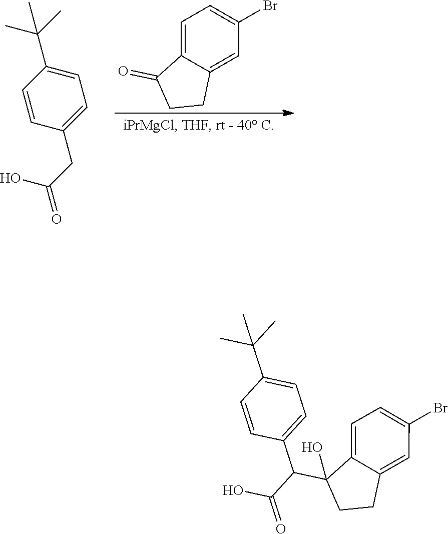

To a solution of (4-tert-butylphenyl)acetic acid (5.4 g, 28.4 mmol) in THF (10 mL) was slowly added i-PrMgBr (30 mL, 30 mmol, 3M in THF) at room temperature. The resulting suspension was stirred at 40° C. for 1 hour. Then, 5-bromo-2,3-dihydroinden-1-one (3.0 g, 14.2 mmol) was added dropwise at 25° C. The resulting solution was stirred at 40° C. for an additional 1 hour. Upon completion, the reaction was quenched with water (50 mL), acidified to pH=2 with conc. HCl and extracted with EA (3×50 mL). The combined organic layers were washed with brine (1×20 mL), dried over anhydrous Na₂SO₄, filtered and concentrated under reduced pressure. The crude residue was purified by reversed-phase flash chromatography (conditions: column, C18 silica gel; mobile phase, ACN in Water (0.1% FA), 0% to 100% gradient in 25 min; detector, UV 254 nm) to afford 2-(5-bromo-1-hydroxy-2,3-dihydro-1H-inden-1-yl)-2-(4-(tert-butyl)phenyl)acetic acid (600 mg) as a white solid. LCMS (ESI) [M+H]⁺: 403/405.

### Step 2: 2-(6-bromo-1H-inden-3-yl)-2-(4-(tert-butyl)phenyl)acetic acid

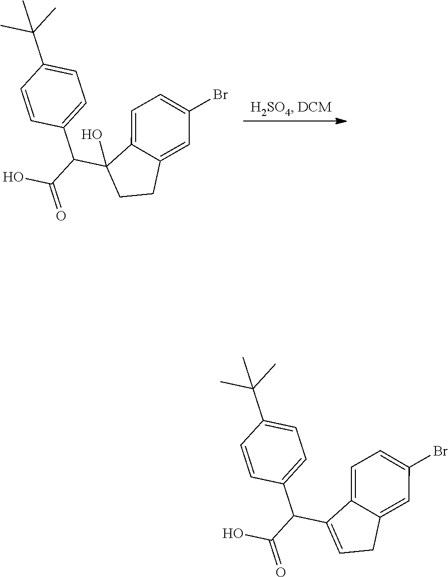 [SMILES: CC(C)(C)c1ccc(C(C(=O)O)C2(O)CCc3cc(Br)ccc32)cc1 ; CC(C)(C)c1ccc(C(C(=O)O)C2=CCc3cc(Br)ccc32)cc1]

H₂SO₄ (0.5 mL, 0.1 mmol) was added to a solution of 2-(5-bromo-1-hydroxy-2,3-dihydro-1H-inden-1-yl)-2-(4-(tert-butyl)phenyl)acetic acid (600 mg, 1.4 mmol) in DCM (5.0 mL) at room temperature. The resulting solution was stirred for 1 hour at room temperature and was then poured into ice water. A white precipitate formed immediately, which was collected by filtration. The precipitate was washed with water, PE and dried under vacuum to afford 2-(6-bromo-1H-inden-3-yl)-2-(4-(tert-butyl)phenyl)acetic acid (500 mg, crude) as a white solid. The crude material was used in the next step directly without further purification. LCMS (ESI) [M+H]⁺: 385/387.

### Step 3: 2-(6-bromo-1H-inden-3-yl)-2-(4-(tert-butyl)phenyl)-N-(2′,4′,6′-trimethyl-[1,1′-biphenyl]-4-yl)acetamide

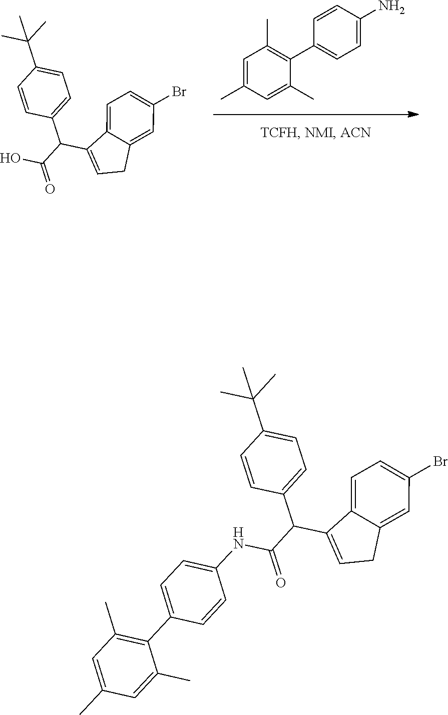 [SMILES: CC(C)(C)c1ccc(C(C(=O)O)C2=CCc3cc(Br)ccc32)cc1 ; Cc1cc(C)c(-c2ccc(N)cc2)c(C)c1 ; Cc1cc(C)c(-c2ccc(NC(=O)C(C3=CCc4cc(Br)ccc43)c3ccc(C(C)(C)C)cc3)cc2)c(C)c1]

A mixture of 2-(6-bromo-1H-inden-3-yl)-2-(4-(tert-butyl)phenyl)acetic acid (600 mg, 1.5 mmol), 2′,4′,6′-trimethyl-[1,1′-biphenyl]-4-amine (658 mg, 3.1 mmol) and NMI (256 mg, 3.1 mmol) in ACN (3.0 mL) was stirred at room temperature for 15 minutes. TCFH (874 mg, 3.1 mmol) was added, and the resulting mixture was stirred at room temperature for 1 hour. Upon completion, the resulting mixture was concentrated under reduced pressure. The crude residue was purified by silica gel column chromatography, eluted with PE/EA (40%) to afford 2-(6-bromo-1H-inden-3-yl)-2-(4-(tert-butyl)phenyl)-N-(2′,4′,6′-trimethyl-[1,1′-biphenyl]-4-yl)acetamide (330 mg, crude) as a yellow solid. LCMS (ESI) [M+H]⁺: 578/580.

### Step 4: 3-(1-(4-(tert-butyl)phenyl)-2-oxo-2-((2′,4′,6′-trimethyl-[1,1′-biphenyl]-4-yl)amino)ethyl)-1H-indene-6-carboxylic acid

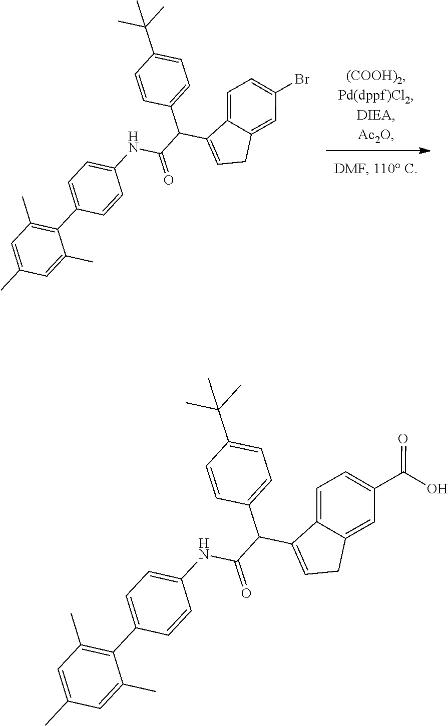 [SMILES: Cc1cc(C)c(-c2ccc(NC(=O)C(C3=CCc4cc(Br)ccc43)c3ccc(C(C)(C)C)cc3)cc2)c(C)c1 ; Cc1cc(C)c(-c2ccc(NC(=O)C(C3=CCc4cc(C(=O)O)ccc43)c3ccc(C(C)(C)C)cc3)cc2)c(C)c1]

A solution 2-(6-bromo-1H-inden-3-yl)-2-(4-(tert-butyl)phenyl)-N-(2′,4′,6′-trimethyl-[1,1′-biphenyl]-4-yl)acetamide (300 mg, 0.52 mmol), Pd(OAc)₂ (10 mg, 0.1 mmol), DIEA (220 mg, 1.04 mmol), 4,5-bis(diphenylphosphino)-9,9-dimethylxanthene (20 mg, 0.2 mmol), anhydrous oxalic acid (90 mg, 1.0 mmol) and acetic anhydride (34 mg, 1 mmol) in DMF (5.0 mL) was stirred at 110° C. for 1 hour under nitrogen atmosphere. Upon completion, the mixture was cooled to room temperature and poured into water (100 mL). The resulting mixture was extracted with EA (3×20 mL). The combined organic layers were washed with H₂O (120 mL), dried over anhydrous Na₂SO₄, filtered and concentrated under reduced pressure. The crude residue was purified by silica gel column chromatography, eluted with PE/EA (1/1) to afford 3-(1-(4-(tert-butyl)phenyl)-2-oxo-2-((2′,4′,6′-trimethyl-[1,1′-biphenyl]-4-yl)amino)ethyl)-1H-indene-6-carboxylic acid (120 mg) as a yellow solid. LCMS (ESI) [M+H]⁺: 544.

### Step 5: 1-(1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxylic acid

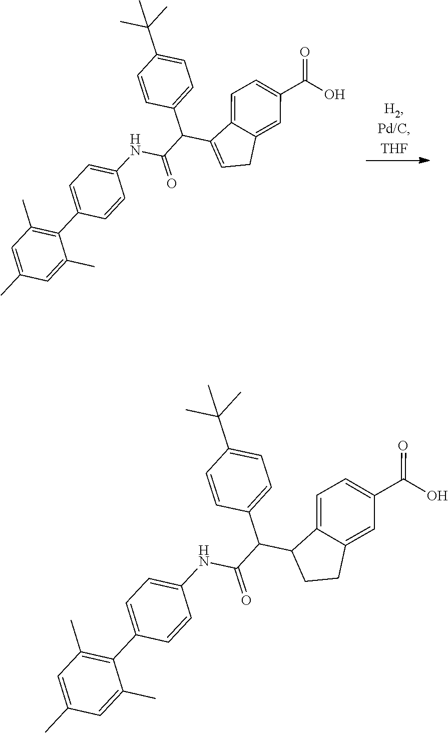 [SMILES: Cc1cc(C)c(-c2ccc(NC(=O)C(C3=CCc4cc(C(=O)O)ccc43)c3ccc(C(C)(C)C)cc3)cc2)c(C)c1 ; Cc1cc(C)c(-c2ccc(NC(=O)C(c3ccc(C(C)(C)C)cc3)C3CCc4cc(C(=O)O)ccc43)cc2)c(C)c1]

A solution of 3-(1-(4-(tert-butyl)phenyl)-2-oxo-2-((2′,4′,6′-trimethyl-[1,1′-biphenyl]-4-yl)amino)ethyl)-1H-indene-6-carboxylic acid (120 mg, 0.2 mmol) and Pd/C (5.8 mg, 0.02 mmol) in THF (5.0 mL) was stirred at room temperature for 2 hours under hydrogen atmosphere. Upon completion, the reaction mixture was filtered over celite. The filter cake was washed with DCM (3×5 mL). The filtrate was concentrated under reduced pressure to afford crude 1-(1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxylic acid (100 mg, crude) as a yellow solid, which was used in the next step directly without further purification. LCMS (ESI) [M+H]⁺: 546.

### Step 6: 2-(1-(1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

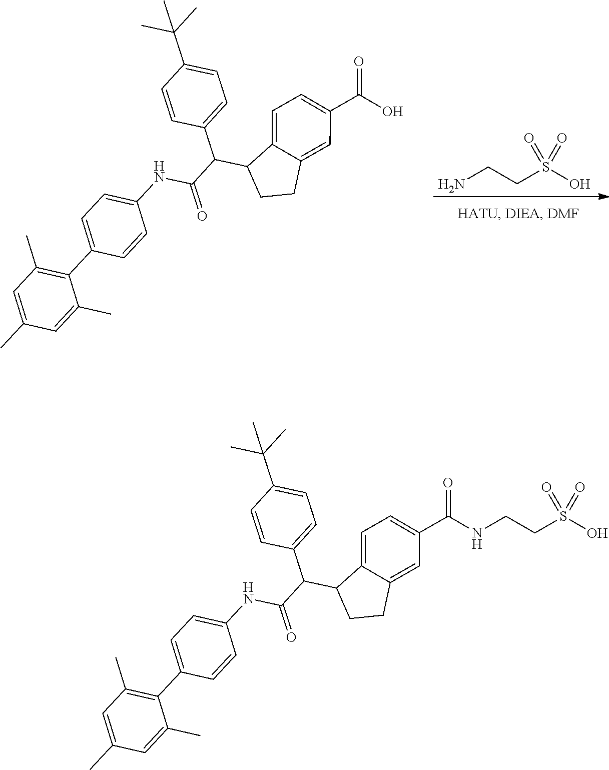 [SMILES: Cc1cc(C)c(-c2ccc(NC(=O)C(c3ccc(C(C)(C)C)cc3)C3CCc4cc(C(=O)O)ccc43)cc2)c(C)c1 ; NCCS(=O)(=O)O ; Cc1cc(C)c(-c2ccc(NC(=O)C(c3ccc(C(C)(C)C)cc3)C3CCc4cc(C(=O)NCCS(=O)(=O)O)ccc43)cc2)c(C)c1]

A solution of 1-(1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxylic acid (100 mg, 0.1 mmol), 2-aminoethanesulfonic acid (46 mg, 0.3 mmol), HATU (139 mg, 0.3 mmol) and DIEA (37 mg, 0.3 mmol) in DMF (3 mL) was stirred at 25° C. for 1 hour. Upon completion, the mixture was poured into water (20 mL) and extracted with EA (3×20 mL). The combined organic layers were washed with water (1×20 mL), dried over anhydrous Na₂SO₄, filtered and concentrated under reduced pressure. The crude residue was purified by reversed-phase flash chromatography (conditions: column, C18 silica gel; mobile phase, ACN in H₂O (0.1% FA), 10% to 50% gradient in 10 min; detector, UV 254 nm) to afford 2-(1-(1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (60 mg) as a white solid. LCMS (ESI) [M+H]⁺: 653.

### Step 7: 2-((S)-1-((R)-1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid, 2-((R)-1-((S)-1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid, 2-((S)-1-((S)-1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((R)-1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

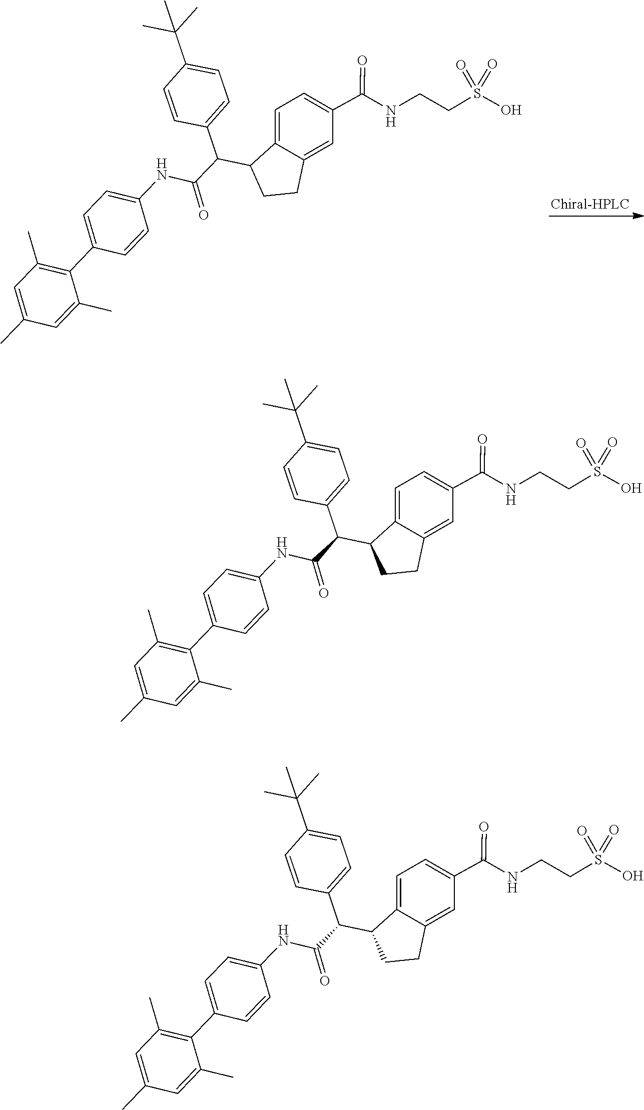 [SMILES: Cc1cc(C)c(-c2ccc(NC(=O)C(c3ccc(C(C)(C)C)cc3)C3CCc4cc(C(=O)NCCS(=O)(=O)O)ccc43)cc2)c(C)c1 ; Cc1cc(C)c(-c2ccc(NC(=O)[C@@H](c3ccc(C(C)(C)C)cc3)[C@@H]3CCc4cc(C(=O)NCCS(=O)(=O)O)ccc43)cc2)c(C)c1 ; Cc1cc(C)c(-c2ccc(NC(=O)[C@H](c3ccc(C(C)(C)C)cc3)[C@H]3CCc4cc(C(=O)NCCS(=O)(=O)O)ccc43)cc2)c(C)c1]

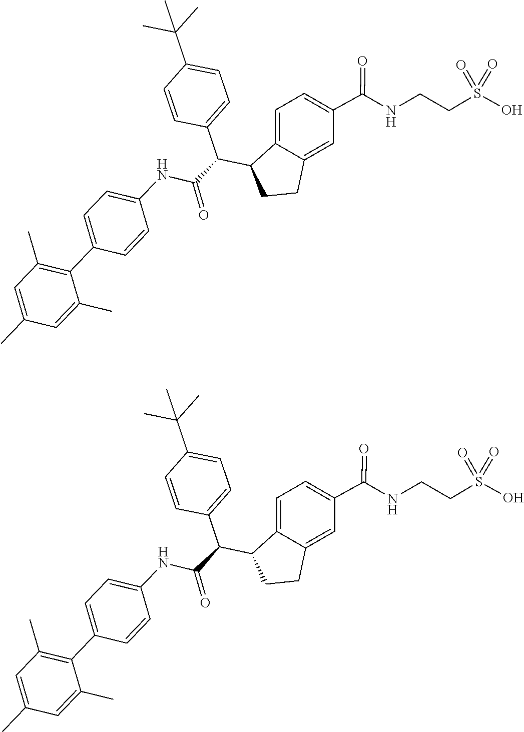 [SMILES: Cc1cc(C)c(-c2ccc(NC(=O)[C@H](c3ccc(C(C)(C)C)cc3)[C@@H]3CCc4cc(C(=O)NCCS(=O)(=O)O)ccc43)cc2)c(C)c1 ; Cc1cc(C)c(-c2ccc(NC(=O)[C@@H](c3ccc(C(C)(C)C)cc3)[C@H]3CCc4cc(C(=O)NCCS(=O)(=O)O)ccc43)cc2)c(C)c1]

2-(1-(1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (60 mg, 0.09 mmol) was separated by chiral HPLC (conditions: Column, (S, S)-WHELK-01, 2*25 cm, 5 μm; Mobile Phase A: Hex, Mobile Phase B: EtOH (0.1% DEA+0.1% FA); Flow rate: 20 mL/min; Gradient: isocratic 50% B; Wave Length: 248/220 nm; RT1 (min): 11.22; RT2 (min): 16.51) to afford fraction 1 (21 mg) and fraction 2 (11 mg), each as a mixture of two diastereomers. Fraction 1 (21 mg) was further separated by Chiral HPLC (conditions: Column, CHIRALPAK IE, 2*25 cm, 5 m; Mobile Phase A: MtBE (10 mM NH₃-MeOH), Mobile Phase B: MeOH; Flow rate: 20 mL/min; Gradient: isocratic 20% B; Wave Length: 220/254 nm; RT1 (min): 9.3; RT2 (min): 12.2) to afford 2-((S)-1-((R)-1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 4a) (Method D, Peak 1, 1.91 min; 3.0 mg, 5.0% yield) and 2-((R)-1-((S)-1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 4b) (Method D, Peak 2, 2.75 min; 8.1 mg, 13.5% yield).

Fraction 2 (11 mg) was further separated by Chiral HPLC (Column: CHIRALPAK IE, 2*25 cm, 5 Hm; Mobile Phase A: MtBE (10 mM NH₃-MeOH), Mobile Phase B: ME H; Flow rate: 20 mL/min; Gradient: isocratic 20% B; Wave Length: 220/254 nm; RT1 (min): 6.5; RT2 (min): 9.0) to afford 2-((S)-1-((S)-1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 4c) (Method D, Peak 1, 1.64 m; 2.7 mg, 4.5% yield) and 2-((R)-1-((R)-1-(4-(tert-butyl)phenyl)-2-oxo-2-((2′,4′,6′-trimethyl-[1,1′-biphenyl]-4-yl)amino)ethyl)-2,3-dihydro-1H-indene-5-carboxamido)ethane-2-sulfonic acid (Compound 4d) (Method D, Peak 2, 8.54 m(s; 2.0 mg, 3.3% yield).

| Compound# | Characterization Data |
|---|---|
| 4a | LCMS (ESI) [M + H]⁺: 653.5 |
| | ¹H NMR (400 MHz, Methanol-d4) δ 7.65 (s, 1H), 7.59 (d, J = 8.6 Hz, 2H), 7.41 (d, J = 8.6 Hz, |
| | 2H), 7.35 (d, J = 8.8 Hz, 2H), 7.25 (d, J = 8.3 Hz, 1H), 7.03 (d, J = 8.6 Hz, 2H), 6.88 (s, 2H), |
| | 6.19 (d, J = 7.9 Hz, 1H), 4.04-3.97 (m, 1H), 3.76 (dd, J = 6.6 Hz, 2H), 3.63 (d, J = 11.0 Hz, |
| | 1H), 3.16-3.09 (m, 1H), 3.05 (dd, J = 6.6 Hz, 2H), 2.99-2.85 (m, 2H), 2.51-2.35 (m, 1H), |
| | 2.27 (s, 3H), 2.17-2.00 (m, 1H), 1.95 (s, 6H), 1.35 (s, 9H). |
| 4b | LCMS (ESI) [M + H]⁺: 653.5 |
| | ¹H NMR (400 MHz, Methanol-d4) δ 7.69 (s, 1H), 7.58 (d, J = 8.0 Hz, 1H), 7.54 (d, J = 6.6 Hz, |
| | 2H), 7.47-7.36 (m, 5H), 7.02 (d, J = 8.5 Hz, 2H), 6.87 (s, 2H), 4.16-4.04 (m, 1H), 3.78 (dd, |
| | J = 6.8 Hz, 2H), 3.61 (d, J = 11.1 Hz, 1H), 3.07 (dd, J = 6.8 Hz, 2H), 3.00-2.93 (m, 1H), 2.91- |
| | 2.78 (m, 1H), 2.27 (s, 3H), 2.08-1.97 (m, 1H), 1.95 (s, 6H), 1.75-1.64 (m, 1H), 1.33 (s, |
| | 9H). |
| 4c | LCMS (ESI) [M + H]⁺: 653.4 |
| | ¹H NMR (400 MHz, Methanol-d4) δ 7.65 (s, 1H), 7.59 (d, J = 8.5 Hz, 2H), 7.41 (d, J = 8.3 Hz, |
| | 2H), 7.35 (d, J = 8.5 Hz, 2H), 7.24 (d, J = 9.3 Hz, 1H), 7.03 (d, J = 8.5 Hz, 2H), 6.87 (s, 2H), |
| | 6.19 (d, J = 7.9 Hz, 1H), 4.04-3.97 (m, 1H), 3.76 (dd, J = 6.6 Hz, 2H), 3.63 (d, J = 11.1 Hz, |
| | 1H), 3.17-3.01 (m, 3H), 3.00-2.88 (m, 1H), 2.52-2.35 (m, 1H), 2.27 (s, 3H), 2.11-2.02 |
| | (m, 1H), 1.95 (s, 6H), 1.34 (s, 9H). |
| 4d | LCMS (ESI) [M + H]⁺: 653.5 |
| | ¹H NMR (400 MHz, Methanol-d4) δ 7.70 (s, 1H), 7.58 (d, J = 7.8 Hz, 1H), 7.54 (d, J = 8.3 Hz, |
| | 2H), 7.49-7.31 (m, 5H), 7.02 (d, J = 8.4 Hz, 2H), 6.87 (s, 2H), 4.14-4.08 (m, 1H), 3.78 (dd, |
| | J = 6.5 Hz, 2H), 3.61 (d, J = 11.1 Hz, 1H), 3.07 (dd, J = 6.6 Hz, 2H), 3.00-2.92 (m, 1H), 2.89- |
| | 2.79 (m, 1H), 2.27 (s, 3H), 2.09-1.98 (m, 1H), 1.95 (s, 6H), 1.75-1.66 (m, 1H), 1.33 (s, |
| | 9H). |

### Example 5: 2-((S)-1-((S)-i-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1l-sulfonic acid and 2-((R)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

### Step 1: 2-(1-((S)-i-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

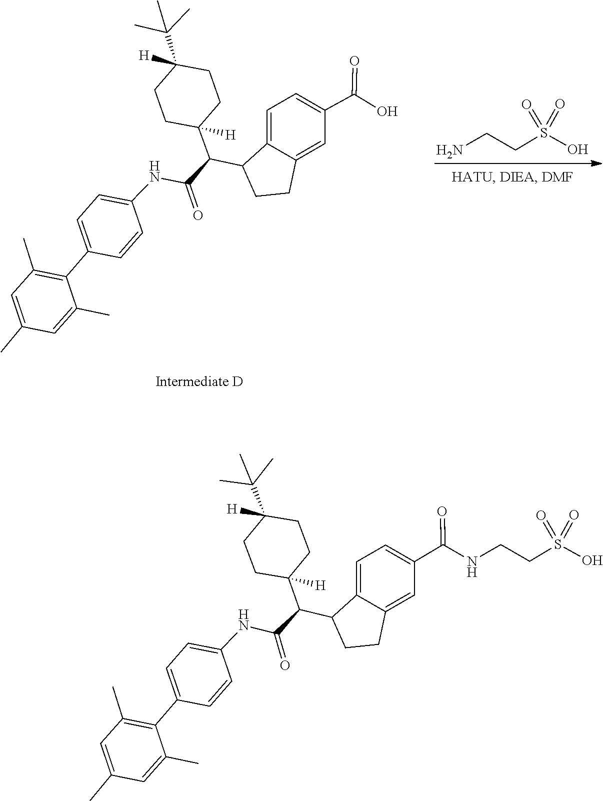 [SMILES: Cc1cc(C)c(-c2ccc(NC(=O)[C@H](C3CCc4cc(C(=O)O)ccc43)[C@H]3CC[C@H](C(C)(C)C)CC3)cc2)c(C)c1 ; NCCS(=O)(=O)O ; Cc1cc(C)c(-c2ccc(NC(=O)[C@H](C3CCc4cc(C(=O)NCCS(=O)(=O)O)ccc43)[C@H]3CC[C@H](C(C)(C)C)CC3)cc2)c(C)c1]

A solution of 1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate D, 300 mg, 0.54 mmol), HATU (310 mg, 0.81 mmol) and DIEA (210 mg, 1.63 mmol) in DMF (3 mL) was stirred at room temperature for 2 hours. Upon completion, the mixture was concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 60% to 70% in 10 min; detector, UV 254 nm) to afford 2-(1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (150 mg, 42% yield) as a white solid. LCMS (ESI) [M+H]⁺: 659.6

### Step 2: 2-((S)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

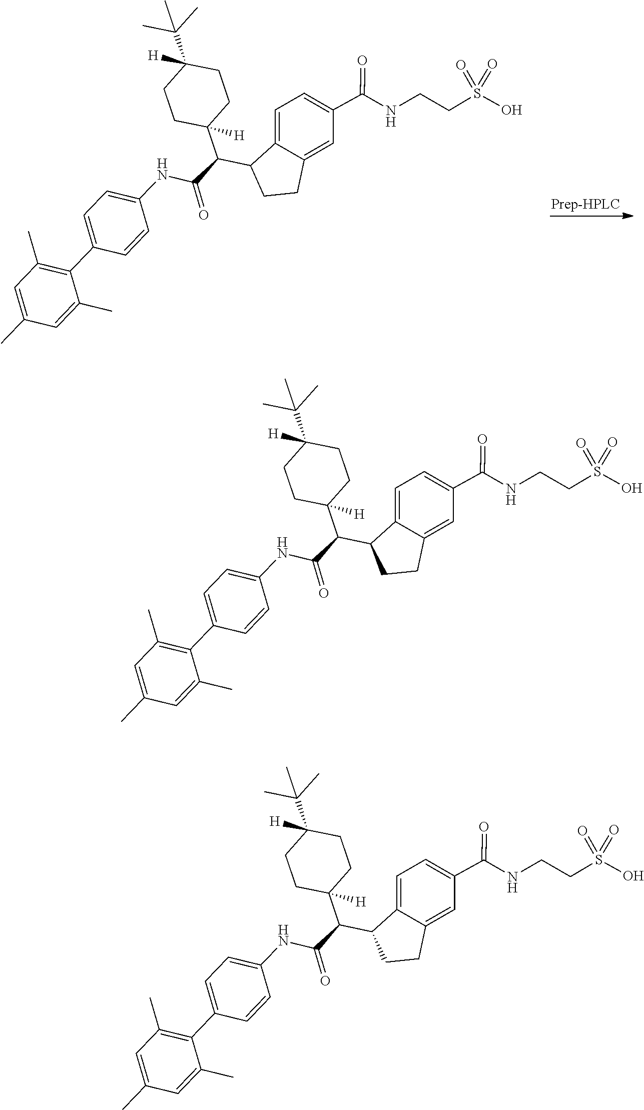 [SMILES: Cc1cc(C)c(-c2ccc(NC(=O)[C@H](C3CCc4cc(C(=O)NCCS(=O)(=O)O)ccc43)[C@H]3CC[C@H](C(C)(C)C)CC3)cc2)c(C)c1 ; Cc1cc(C)c(-c2ccc(NC(=O)[C@@H]([C@H]3CC[C@H](C(C)(C)C)CC3)[C@@H]3CCc4cc(C(=O)NCCS(=O)(=O)O)ccc43)cc2)c(C)c1 ; Cc1cc(C)c(-c2ccc(NC(=O)[C@@H]([C@H]3CC[C@H](C(C)(C)C)CC3)[C@H]3CCc4cc(C(=O)NCCS(=O)(=O)O)ccc43)cc2)c(C)c1]

2-(1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (150 mg) was purified by Prep-HPLC (conditions: Column: XBridge Prep OBD C18 Column 30*150 mm, 5 m; Mobile Phase A: Water (10 mmol/L NH₄HCO₃), Mobile Phase B: MeCN; Flow rate: 60 mL/min; Gradient (B %): 50% B to 70% B in 15 min; Wave Length: 254 nm/220 nm; RT1 (min): 10.45; RT2 (min): 11.45) to afford 2-((S)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 5a) (15.6 mg, 10% yield) and 2-((R)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 5b) (39.7 mg, 26% yield) as a white solid.

| Compound# | Characterization Data |
|---|---|
| 5a | LCMS (ESI) [M − H]⁻: 657.3 |
| | ¹H NMR (400 MHz, Methanol-d4) δ 7.64 (s, 1H), 7.59 (d, J = 8.0 Hz, 1H), 7.49 (d, J = 8.5 Hz, |
| | 2H), 7.41 (d, J = 8.0 Hz, 1H), 7.01 (d, J = 8.6 Hz, 2H), 6.88 (s, 2H), 3.78 (t, J = 6.7 Hz, 2H), |
| | 3.64-3.60 (m, 1H), 3.10-2.96 (m, 4H), 2.89-2.82 (m, 1H), 2.42-2.34 (m, 2H), 2.29- |
| | 2.21 (m, 4H), 1.95 (s, 6H), 1.82-1.77 (m, 1H), 1.65-1.50 (m, 4H), 1.44-1.37 (m, 1H), 1.27- |
| | 1.22 (m, 2H), 1.11-1.03 (m, 1H), 0.89 (s, 9H). |
| 5b | LCMS (ESI) [M − H]⁻: 657.4 |
| | ¹H NMR (400 MHz, Methanol-d4) δ 7.66 (d, J = 7.9 Hz, 1H), 7.60 (s, 1H), 7.53 (d, J = 8.1 Hz, |
| | 1H), 7.24-7.18 (m, 2H), 6.91 (d, J = 8.6 Hz, 2H), 6.85 (s, 2H), 3.76 (t, J = 6.7 Hz, 2H), 3.68- |
| | 3.62 (m, 1H), 3.25 (dd, J = 11.3, 4.6 Hz, 1H), 3.11-2.95 (m, 3H), 2.88-2.79 (m, 1H), 2.72- |
| | 2.56 (m, 1H), 2.36 (d, J = 11.2 Hz, 1H), 2.27-2.17 (m, 4H), 2.10 (d, J = 13.7 Hz, 1H), 1.90 (s, |
| | 6H), 1.81-1.47 (m, 6H), 1.33-1.24 (m, 1H), 1.13 (t, J = 11.1 Hz, 1H), 0.95 (s, 9H). |

### Example 6: 2-((S)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

### Step 1: 2-(1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

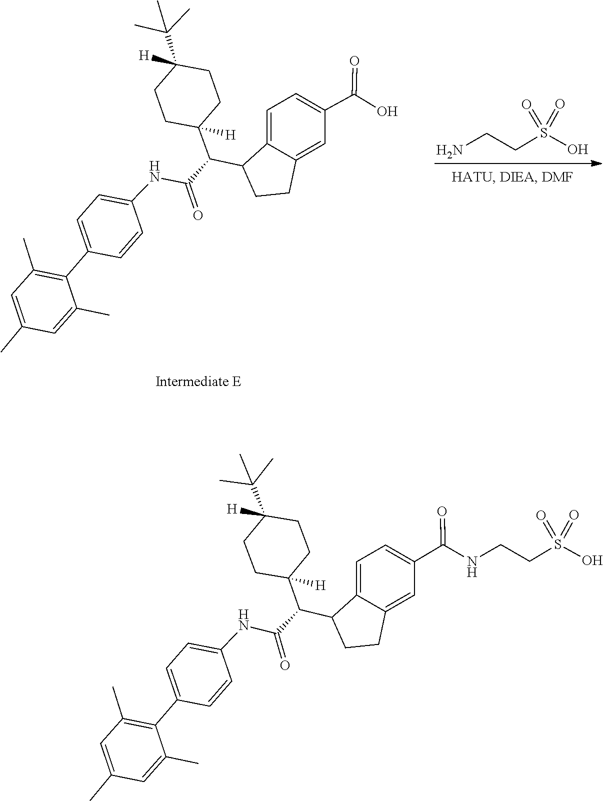 [SMILES: Cc1cc(C)c(-c2ccc(NC(=O)[C@@H](C3CCc4cc(C(=O)O)ccc43)[C@H]3CC[C@H](C(C)(C)C)CC3)cc2)c(C)c1 ; NCCS(=O)(=O)O ; Cc1cc(C)c(-c2ccc(NC(=O)[C@@H](C3CCc4cc(C(=O)NCCS(=O)(=O)O)ccc43)[C@H]3CC[C@H](C(C)(C)C)CC3)cc2)c(C)c1]

A solution of 1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate E, 300 mg, 0.54 mmol), HATU (310 mg, 0.81 mmol) and DIEA (210 mg, 1.63 mmol) in DMF (3 mL) was stirred at room temperature for 2 hours. Upon completion, the reaction mixture was concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 60% to 70% in 10 min; detector, UV 254 nm) to give 2-(1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (154 mg, 42% yield) as a white solid. LCMS (ESI) [M+H]⁺: 659.1

### Step 2: 2-((S)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

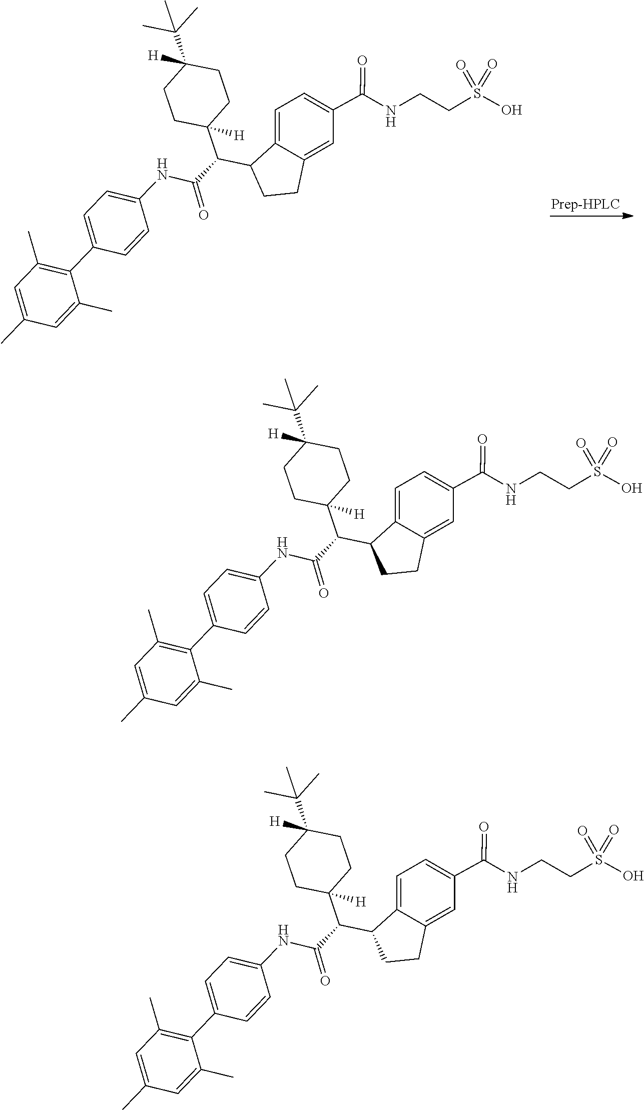 [SMILES: Cc1cc(C)c(-c2ccc(NC(=O)[C@@H](C3CCc4cc(C(=O)NCCS(=O)(=O)O)ccc43)[C@H]3CC[C@H](C(C)(C)C)CC3)cc2)c(C)c1 ; Cc1cc(C)c(-c2ccc(NC(=O)[C@H]([C@H]3CC[C@H](C(C)(C)C)CC3)[C@@H]3CCc4cc(C(=O)NCCS(=O)(=O)O)ccc43)cc2)c(C)c1 ; Cc1cc(C)c(-c2ccc(NC(=O)[C@H]([C@H]3CC[C@H](C(C)(C)C)CC3)[C@H]3CCc4cc(C(=O)NCCS(=O)(=O)O)ccc43)cc2)c(C)c1]

2-(1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (154 mg) was purified by Prep-HPLC (conditions: Column: XBridge Prep OBD C18 Column 30*150 mm, 5 μm; Mobile Phase A: Water (10 mmol/L NH₄HCO₃), Mobile Phase B: MeCN; Flow rate: 60 mL/min; Gradient (B %): 49% B to 69% B in 15 min; Wave Length: 254 nm/220 nm; RT1 (min): 9.28; RT2 (min): 10.02) to afford 2-((S)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 6a) (10.4 mg, 7% yield) and 2-((R)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 6b) (58.3 mg, 37% yield) as a white solid.

| Compound# | Characterization Data |
|---|---|
| 6a | LCMS (ESI) [M + H]⁺: 659.4 |
| | ¹H NMR (400 MHz, Methanol-d4) δ 7.64 (s, 1H), 7.59 (d, J = 8.0 Hz, 1H), 7.49 (d, J = 8.6 Hz, |
| | 2H), 7.41 (d, J = 8.0 Hz, 1H), 7.01 (d, J = 8.6 Hz, 2H), 6.88 (s, 2H), 3.78 (t, J = 6.7 Hz, 2H), |
| | 3.65-3.56 (m, 1H), 3.11-2.95 (m, 4H), 2.91-2.81 (m, 1H), 2.42-2.33 (m, 2H), 2.28- |
| | 2.21 (m, 4H), 1.95 (s, 6H), 1.79 (d, J = 13.4 Hz, 1H), 1.66-1.51 (m, 4H), 1.47-1.37 (m, 1H), |
| | 1.33-1.26 (m, 2H), 1.13-1.07 (m, 1H), 0.89 (s, 9H). |
| 6b | LCMS (ESI) [M + H]⁺: 659.4 |
| | ¹H NMR (400 MHz, Methanol-d4) δ 7.66 (d, J = 8.0 Hz, 1H), 7.60 (s, 1H), 7.53 (d, J = 8.0 Hz, |
| | 1H), 7.25-7.18 (m, 2H), 6.91 (d, J = 8.6 Hz, 2H), 6.85 (s, 2H), 3.75 (t, J = 6.8 Hz, 2H), 3.71- |
| | 3.61 (m, 1H), 3.25 (dd, J = 11.3, 4.6 Hz, 1H), 3.07-2.98 (m, 3H), 2.88-2.80 (m, 1H), 2.71- |
| | 2.60 (m, 1H), 2.39-2.33 (m, 1H), 2.25-2.16 (m, 4H), 2.13-2.08 (m, 1H), 1.90 (s, 6H), 1.81- |
| | 1.50 (m, 6H), 1.33-1.25 (m, 1H), 1.17-1.09 (m, 1H), 0.95 (s, 9H). |

### Example 7: 2-((S)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

### Step 1: 2-(1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

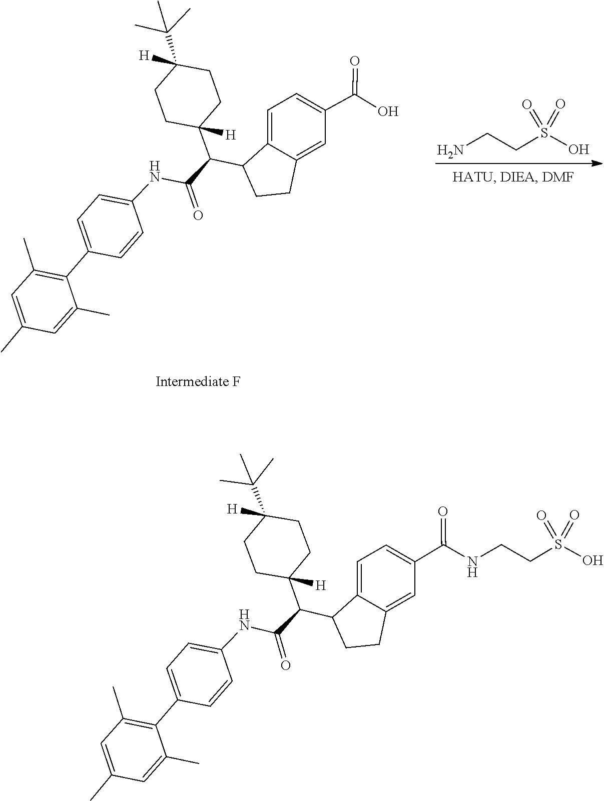 [SMILES: Cc1cc(C)c(-c2ccc(NC(=O)[C@H](C3CCc4cc(C(=O)O)ccc43)[C@H]3CC[C@@H](C(C)(C)C)CC3)cc2)c(C)c1 ; NCCS(=O)(=O)O ; Cc1cc(C)c(-c2ccc(NC(=O)[C@H](C3CCc4cc(C(=O)NCCS(=O)(=O)O)ccc43)[C@H]3CC[C@@H](C(C)(C)C)CC3)cc2)c(C)c1]

A solution of 1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate F, 60 mg, 0.1 mmol), 2-aminoethanesulfonic acid (68.0 mg, 0.5 mmol), HATU (82.6 mg, 0.2 mmol) and DIEA (22.0 mg, 0.2 mmol) in DMF (1 mL) was stirred at room temperature for 1 hour. Upon completion, the resulting mixture was diluted with water (10 mL) and extracted with EtOAc (3×20 mL). The combined organic layers were washed with water (1×20 mL), dried over anhydrous Na₂SO₄, filtered and concentrated under reduced pressure. The crude residue was purified by reversed-phase flash chromatography (conditions: column, C18 silica gel; mobile phase A: water (0.1% FA), Mobile phase B: MeCN; Gradient (B %): 10% to 50% in 10 min; detector, UV 254 nm) to afford 2-(1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (30 mg, crude) as a white solid. LCMS (ESI) [M+H]⁺: 659.3

### Step 2: 2-((S)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

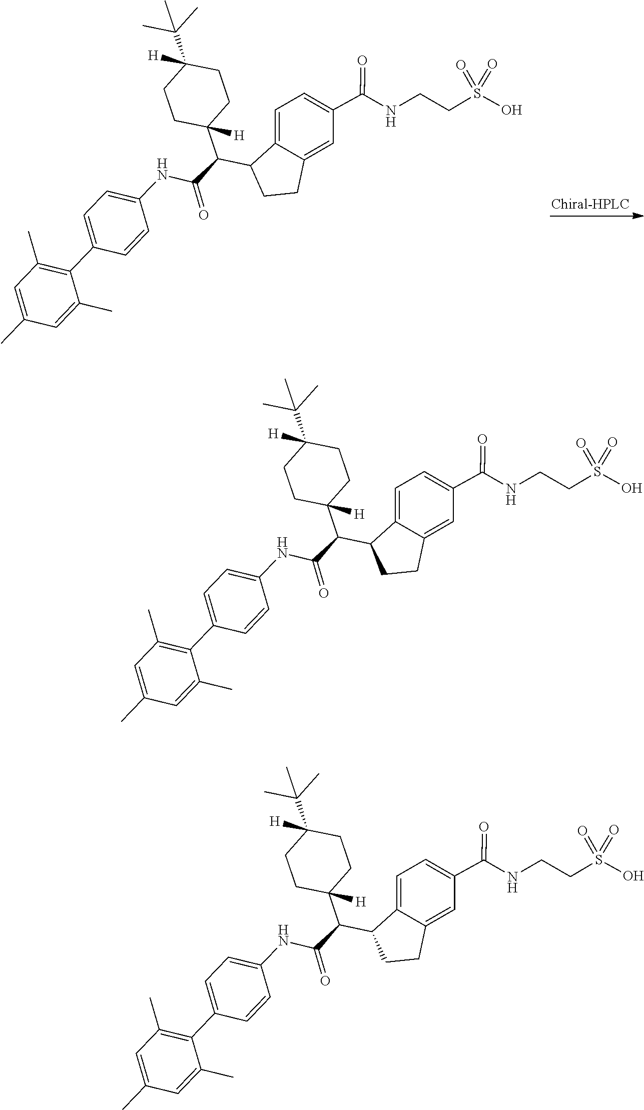 [SMILES: Cc1cc(C)c(-c2ccc(NC(=O)[C@H](C3CCc4cc(C(=O)NCCS(=O)(=O)O)ccc43)[C@H]3CC[C@@H](C(C)(C)C)CC3)cc2)c(C)c1 ; Cc1cc(C)c(-c2ccc(NC(=O)[C@@H]([C@H]3CC[C@@H](C(C)(C)C)CC3)[C@@H]3CCc4cc(C(=O)NCCS(=O)(=O)O)ccc43)cc2)c(C)c1 ; Cc1cc(C)c(-c2ccc(NC(=O)[C@@H]([C@H]3CC[C@@H](C(C)(C)C)CC3)[C@H]3CCc4cc(C(=O)NCCS(=O)(=O)O)ccc43)cc2)c(C)c1]

2-(1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (30 mg, 0.04 mmol) was separated by prep chiral HPLC (conditions: Column: CHIRALPAK ID, 3*25 cm, 5 m; Mobile Phase A: Hex (0.1% 2M NH₃-MeOH), Mobile Phase B: EtOH (0.1% TFA/0.1% DEA); Flow rate: 40 mL/min; Gradient (B %): isocratic 25% B; Wave Length: 210/248 nm; RT1 (min): 7; RT2 (min): 10.5) to afford 2-((S)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 7a) (12.6 mg, 42% yield) and 2-((R)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 7b) (6.8 mg, 23% yield).

| Compound# | Characterization Data |
|---|---|
| 7a | LCMS (ESI) [M − H]⁻: 657.2 |
| | ¹H NMR (400 MHz, Methanol-d4) δ 7.63 (d, J = 5.5 Hz, 2H), 7.44 (d, J = 8.5 Hz, 1H), 7.35 (d, |
| | J = 8.5 Hz, 2H), 6.96 (d, J = 8.4 Hz, 2H), 6.86 (s, 2H), 3.77 (t, J = 6.7 Hz, 2H), 3.73-3.66 (m, |
| | 1H), 3.06 (t, J = 6.5 Hz, 3H), 2.91-2.82 (m, 1H), 2.65-2.59 (m, 1H), 2.56-2.44 (m, 1H), |
| | 2.30-2.18 (m, 4H), 2.11-1.98 (m, 2H), 1.92 (s, 6H), 1.92-1.83 (m, 2H) 1.80-1.70 (m, |
| | 1H), 1.33-1.00 (m, 5H), 0.88 (s, 9H). |
| 7b | LCMS (ESI) [M − H]⁻: 657.2 |
| | ¹H NMR (400 MHz, Methanol-d4) δ 7.66 (s, 1H), 7.57 (d, J = 6.5 Hz, 2H), 7.53 (d, J = 9.5 Hz, |
| | 1H), 7.29 (d, J = 8.0 Hz, 1H), 7.05 (d, J = 8.6 Hz, 2H), 6.89 (s, 2H), 3.77 (t, J = 6.7 Hz, 2H), |
| | 3.74-3.66 (m, 1H), 3.06 (t, J = 6.6 Hz, 2H), 3.03-2.96 (m, 1H), 2.95-2.83 (m, 1H), 2.50 |
| | (dd, J = 9.7, 5.5 Hz, 1H), 2.33 (dt, J = 13.5, 7.4 Hz, 1H), 2.28 (s, 3H), 2.14-1.95 (m, 2H), |
| | 1.98 (s, 6H), 1.94-1.73 (m, 4H), 1.45-0.92 (m, 6H), 0.86 (s, 9H). |

### Example 8: 2-((S)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

### Step 1: 2-(1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

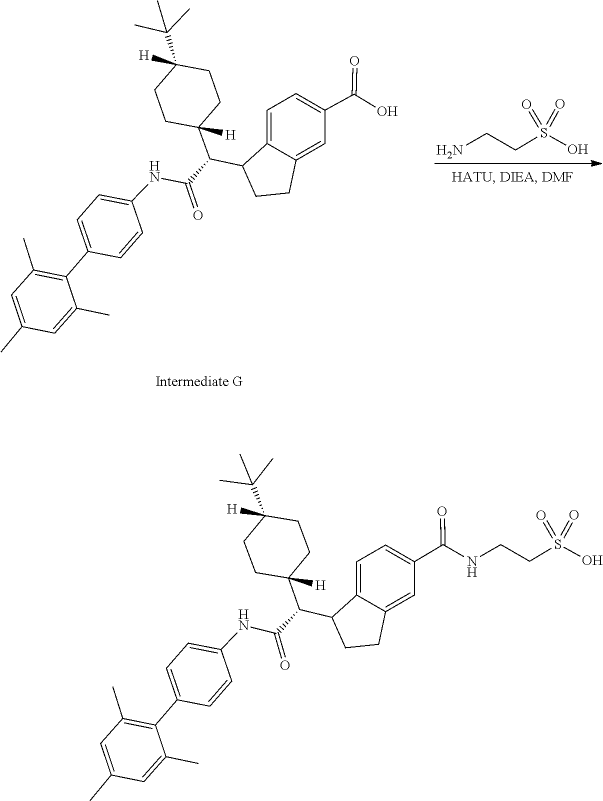 [SMILES: Cc1cc(C)c(-c2ccc(NC(=O)[C@@H](C3CCc4cc(C(=O)O)ccc43)[C@H]3CC[C@@H](C(C)(C)C)CC3)cc2)c(C)c1 ; NCCS(=O)(=O)O ; Cc1cc(C)c(-c2ccc(NC(=O)[C@@H](C3CCc4cc(C(=O)NCCS(=O)(=O)O)ccc43)[C@H]3CC[C@@H](C(C)(C)C)CC3)cc2)c(C)c1]

A solution of 1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate G, 120 mg, 0.2 mmol), 2-aminoethanesulfonic acid (136 mg, 1.0 mmol), HATU (165 mg, 0.4 mmol) and DIEA (44 mg, 0.5 mmol) in DMF (10 mL) was stirred at room temperature for 1 hour. Upon completion, the resulting mixture was diluted with water (10 mL) and extracted with EtOAc (3×30 mL). The combined organic layers were washed with water (1×20 mL), dried over anhydrous Na₂SO₄, filtered and concentrated under reduced pressure. The crude residue was purified by reverse-phase flash chromatography (conditions: column, C18 silica gel; mobile phase A: water (0.1% FA), mobile phase B: MeCN; Gradient (B %): 10% to 50% in 10 min; detector, UV 254 nm) to yield 2-(1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (48 mg, 34% yield) as a white solid. LCMS (ESI) [M+H]⁺: 659.2

### Step 2: 2-((S)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

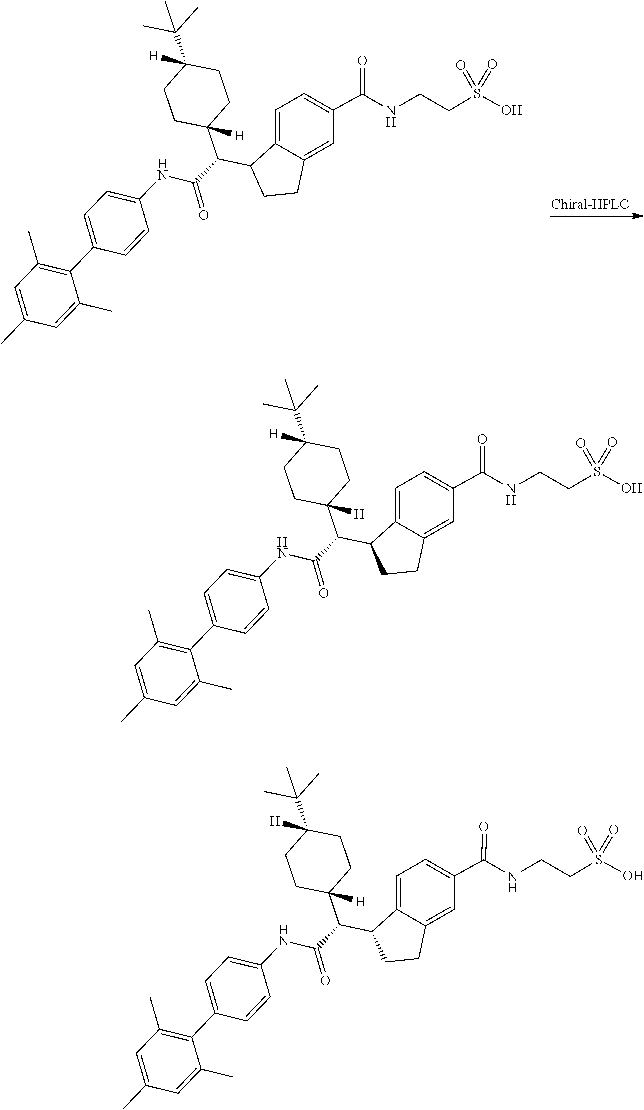 [SMILES: Cc1cc(C)c(-c2ccc(NC(=O)[C@@H](C3CCc4cc(C(=O)NCCS(=O)(=O)O)ccc43)[C@H]3CC[C@@H](C(C)(C)C)CC3)cc2)c(C)c1 ; Cc1cc(C)c(-c2ccc(NC(=O)[C@H]([C@H]3CC[C@@H](C(C)(C)C)CC3)[C@@H]3CCc4cc(C(=O)NCCS(=O)(=O)O)ccc43)cc2)c(C)c1 ; Cc1cc(C)c(-c2ccc(NC(=O)[C@H]([C@H]3CC[C@@H](C(C)(C)C)CC3)[C@H]3CCc4cc(C(=O)NCCS(=O)(=O)O)ccc43)cc2)c(C)c1]

2-(1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (48 mg, 0.64 mmol) was separated by prep-Chiral HPLC (conditions: Column: CHIRALPAK IA, 3*25 cm, 5 μm; Mobile Phase A: Hex (0.1% DEA), Mobile Phase B: EtOH (0.1% TFA/0.1% DEA); Flow rate: 40 mL/min; Gradient (B %): isocratic 30% B; Wave Length: 210/220 nm; RT1 (min): 9.8; RT2 (min): 19) to afford 2-((S)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 8a) (12.9 mg, 27% yield) and 2-((R)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 8b) (5.5 mg, 12% yield).

| Compound# | Characterization Data |
|---|---|
| 8a | LCMS (ESI) [M − H]⁻: 657.2 |
| | ¹H NMR (400 MHz, Methanol-d4) δ 7.63 (d, J = 4.9 Hz, 2H), 7.44 (d, J = 8.0 Hz, 1H), 7.35 (d, |
| | J = 8.6 Hz, 2H), 6.96 (d, J = 8.8 Hz, 2H), 6.86 (s, 2H), 3.77 (t, J = 6.6 Hz, 2H), 3.75-3.65 (m, |
| | 1H), 3.13-3.03 (m, 3H), 2.92-2.79 (m, 1H), 2.62 (t, J = 7.5 Hz, 1H), 2.55-2.46 (m, 1H), |
| | 2.29-2.17 (m, 4H), 2.08-1.95 (m, 4H), 1.92 (s, 6H), 1.78 (d, J = 10.7 Hz, 1H), 1.31-1.06 |
| | (m, 5H), 0.88 (s, 9H). |
| 8b | LCMS (ESI) [M − H]⁻: 657.3 |
| | ¹H NMR (400 MHz, Methanol-d4) δ 7.66 (s, 1H), 7.57 (d, J = 8.6 Hz, 2H), 7.53 (d, J = 8.0 Hz, |
| | 1H), 7.29 (d, J = 8.1 Hz, 1H), 7.05 (d, J = 8.6 Hz, 2H), 6.89 (s, 2H), 3.77 (t, J = 6.6 Hz, 2H), |
| | 3.74-3.63 (m, 1H), 3.06 (t, J = 6.8 Hz, 2H), 3.03-2.96 (m, 1H), 2.93-2.82 (m, 1H), 2.50 |
| | (dd, J = 9.6, 5.5 Hz, 1H), 2.36-2.26 (m, 4H), 2.10-1.95 (m, 8H), 1.92-1.79 (m, 4H), 1.45- |
| | 0.92 (m, 5H), 0.86 (s, 9H). |

### Example 9: 3-((S)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid and 3-((R)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

### Step 1: methyl 3-(1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate

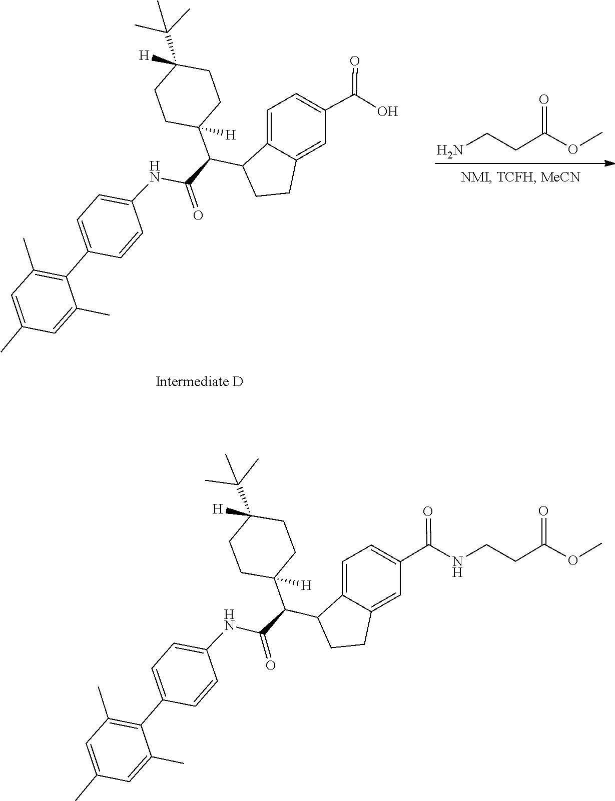

A mixture of 1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate D; 100 mg, 0.18 mmol), methyl 3-aminopropanoate (22 mg, 0.21 mmol), TCFH (76 mg, 0.27 mmol) and 1-methyl-1H-imidazole (45 mg, 0.54 mmol) in MeCN (1 mL) was stirred at room temperature for 3 hours. Upon completion, the mixture was concentrated under reduced pressure. The crude residue was purified by reversed-phase flash chromatography (conditions: column, C18 silica gel; mobile phase A: Water (0.1% FA), Mobile Phase B: MeCN; Gradient (B %): 60% to 70% in 10 min; detector, UV 254 nm) to give methyl 3-(1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate (100 mg, 89%) as a white solid. LCMS (ESI) [M+H]⁺: 637.3

### Step 2: 3-(1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

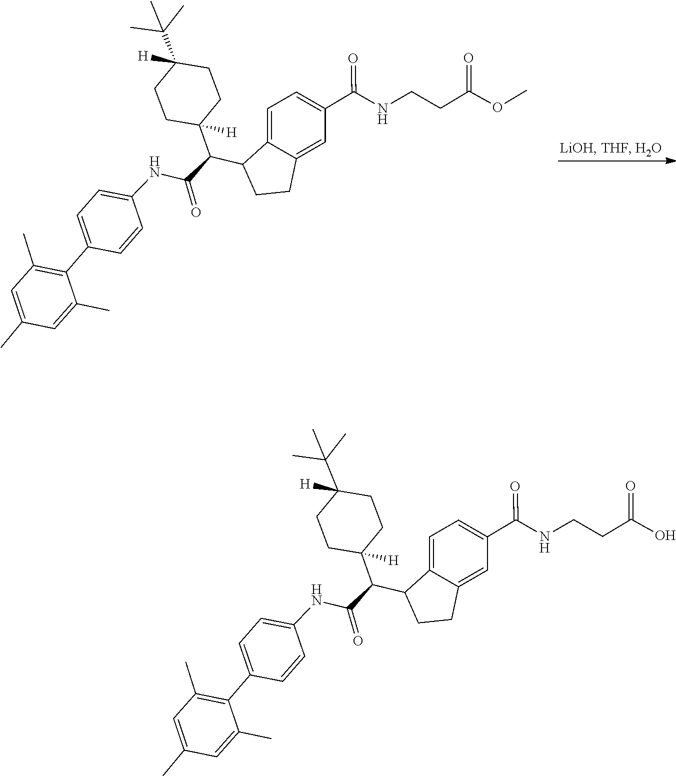

A mixture of methyl 3-(1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate (100 mg, 0.15 mmol) and LiOH monohydrate (37 mg, 1.57 mmol) in THF (2 mL) and H₂O (1 mL) was stirred at room temperature for 3 hours. Upon completion, the mixture was concentrated under reduced pressure. The crude residue was purified by reversed-phase flash chromatography (conditions: column, C18 silica gel; mobile phase A: Water (0.1% FA), Mobile phase B: MeCN; Gradient (B %): 60% to 70% in 10 min; detector, UV 254 nm) to give 3-(1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (65 mg, 66% yield) as a white solid. LCMS (ESI) [M+H]⁺: 623.3

### Step 3: 3-((S)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid and 3-((R)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

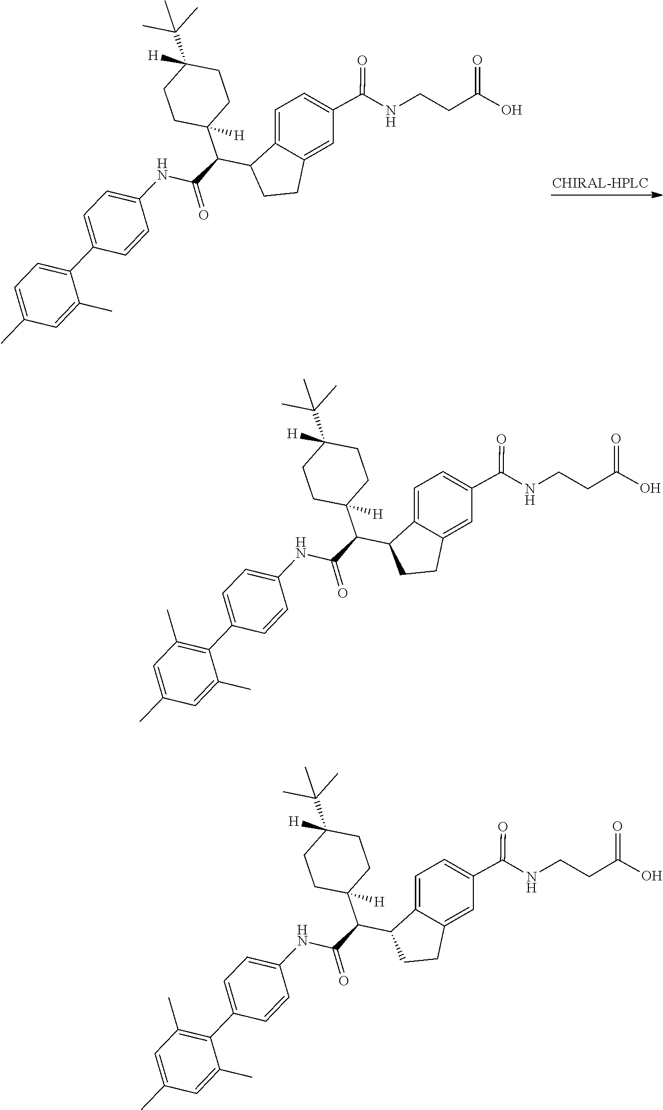 [SMILES: Cc1ccc(-c2ccc(NC(=O)[C@H](C3CCc4cc(C(=O)NCCC(=O)O)ccc43)[C@H]3CC[C@H](C(C)(C)C)CC3)cc2)c(C)c1 ; Cc1cc(C)c(-c2ccc(NC(=O)[C@@H]([C@H]3CC[C@H](C(C)(C)C)CC3)[C@@H]3CCc4cc(C(=O)NCCC(=O)O)ccc43)cc2)c(C)c1 ; Cc1cc(C)c(-c2ccc(NC(=O)[C@@H]([C@H]3CC[C@H](C(C)(C)C)CC3)[C@H]3CCc4cc(C(=O)NCCC(=O)O)ccc43)cc2)c(C)c1]

3-(1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (65 mg) was separated by Prep-Chiral HPLC (conditions: Column: CHIRAL ART Cellulose-SZ, 3*25 cm, 5 μm; Mobile Phase A: Hex (0.1% FA), Mobile Phase B: EtOH; Flow rate: 40 mL/min; Gradient (B %): isocratic 10% B; Wave Length: 205/230 nm; RT1 (min): 8.89; RT2 (min): 13.99) to afford 3-((S)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (Compound 9a) (1^{st} fraction, 46.6 mg, 47%) and 3-((R)-1-((S)-1-((1r,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (Compound 9b) (2^{nd} fraction, 12.2 mg, 16%) as a white solid.

| Compound# | Characterization Data |
|---|---|
| 9a | LCMS (ESI) [M − H]⁻: 623.5 |
| | ¹H NMR (400 MHz, Methanol-d4) δ 7.65 (d, J = 7.9 Hz, 1H), 7.58 (s, 1H), 7.53 (d, J = 7.9 Hz, |
| | 1H), 7.22 (d, J = 8.5 Hz, 2H), 6.91 (d, J = 8.5 Hz, 2H), 6.85 (s, 2H), 3.70-3.62 (m, 1H), 3.57 |
| | (t, J = 6.9 Hz, 2H), 3.25 (dd, J = 11.4, 4.7 Hz, 1H), 3.07-2.99 (m, 1H), 2.88-2.78 (m, 1H), |
| | 2.72-2.63 (m, 1H), 2.58 (t, J = 6.9 Hz, 2H), 2.41-2.32 (m, 1H), 2.25 (s, 3H), 2.23-2.17 (m, |
| | 1H), 2.14-2.07 (m, 1H), 1.90 (s, 6H), 1.80-1.49 (m, 6H), 1.34-1.24 (m, 1H), 1.13 (t, J = |
| | 11.2 Hz, 1H), 0.95 (s, 9H). |
| 9b | LCMS (ESI) [M − H]⁻: 623.5 |
| | ¹H NMR (400 MHz, Methanol-d4) δ 7.62 (s, 1H), 7.57 (d, J = 7.9 Hz, 1H), 7.49 (d, J = 8.5 Hz, |
| | 2H), 7.40 (d, J = 7.9 Hz, 1H), 7.01 (d, J = 8.5 Hz, 2H), 6.88 (s, 2H), 3.68-3.55 (m, 3H), 3.07 |
| | (dd, J = 10.8, 5.1 Hz, 1H), 3.03-2.94 (m, 1H), 2.89-2.79 (m, 1H), 2.61 (t, J = 6.9 Hz, 2H), |
| | 2.45-2.33 (m, 2H), 2.30-2.20 (m, 4H), 2.05-1.98 (m, 1H), 1.95 (s, 6H), 1.82-1.72 (m, |
| | 1H), 1.65-1.47 (m, 4H), 1.47-1.35 (m, 1H), 1.31-1.22 (m, 1H), 1.14-1.04 (m, 1H), 0.90 |
| | (s, 9H). |

### Example 10: 3-((S)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid and 3-((R)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

### Step 1: methyl 3-(1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate

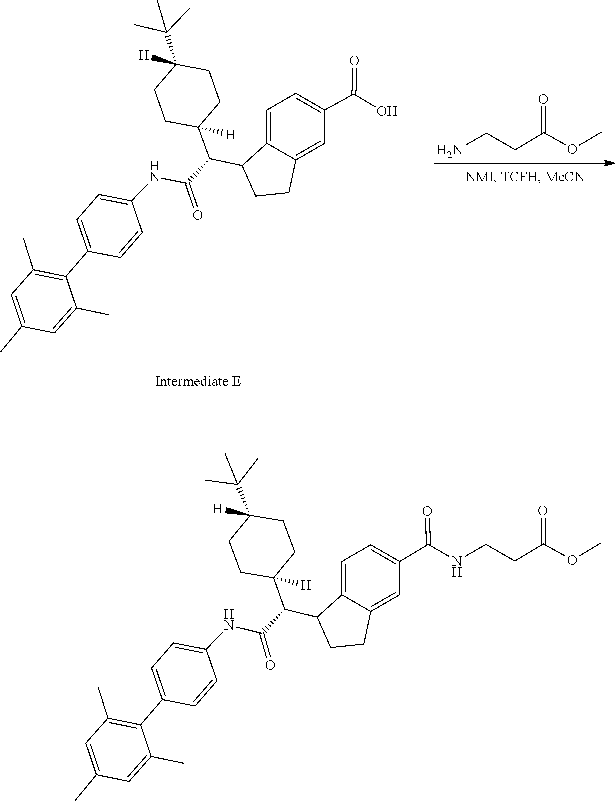

A mixture of 1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate E, 200 mg, 0.4 mmol), methyl 3-aminopropanoate (44 mg, 0.4 mmol), TCFH (160 mg, 0.54 mmol) and 1-methyl-1H-imidazole (88 mg, 0.543 mmol) in MeCN (1 mL) was stirred at room temperature for 3 hours. Upon completion, the mixture was concentrated under reduced pressure. The crude residue was purified by reversed-phase flash chromatography (conditions: column, C18 silica gel; mobile phase A: Water (0.1% FA), Mobile Phase B: MeCN; Gradient (B %): 60% to 70% in 10 min; detector, UV 254 nm) to afford methyl 3-(1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate (180 mg, 71% yield) as a white solid. LCMS (ESI) [M+H]⁺: 636.3

### Step 2: 3-(1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

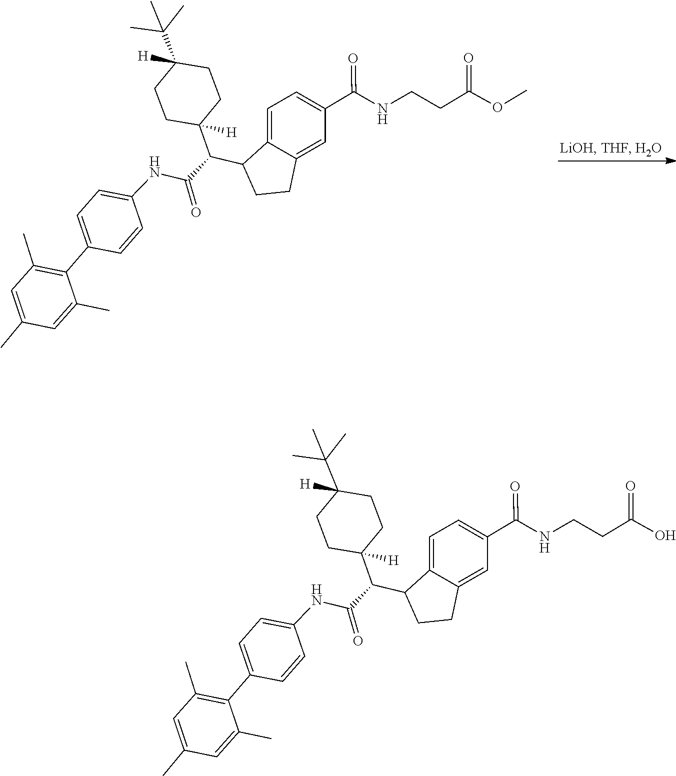

A mixture of methyl 3-(1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate (180 mg, 0.3 mmol) and LiOH monohydrate (80 mg, 2 mmol) in THF (2 mL) and H₂O (1 mL) was stirred at room temperature for 3 hours. Upon completion, the mixture was concentrated under reduced pressure. The crude residue was purified by reversed-phase flash chromatography (conditions: column, C18 silica gel; mobile phase A: Water (0.1% FA), Mobile Phase B: MeCN; Gradient (B %): 60% to 70% in 10 min; detector, UV 254 nm) to give 3-(1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (102 mg, 53% yield) as a white solid. LCMS (ESI) [M+H]⁺: 623.3

### Step 3: 3-((S)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid and 3-((R)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

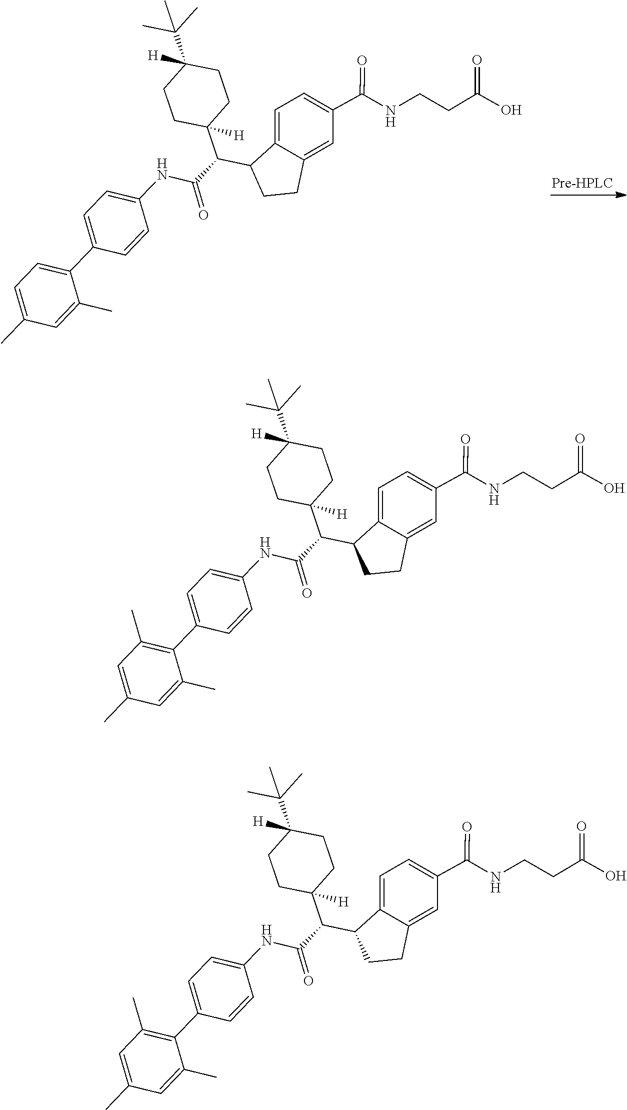 [SMILES: Cc1ccc(-c2ccc(NC(=O)[C@@H](C3CCc4cc(C(=O)NCCC(=O)O)ccc43)[C@H]3CC[C@H](C(C)(C)C)CC3)cc2)c(C)c1 ; Cc1cc(C)c(-c2ccc(NC(=O)[C@H]([C@H]3CC[C@H](C(C)(C)C)CC3)[C@@H]3CCc4cc(C(=O)NCCC(=O)O)ccc43)cc2)c(C)c1 ; Cc1cc(C)c(-c2ccc(NC(=O)[C@H]([C@H]3CC[C@H](C(C)(C)C)CC3)[C@H]3CCc4cc(C(=O)NCCC(=O)O)ccc43)cc2)c(C)c1]

3-(1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (102 mg) was separated by Prep-HPLC (conditions: Column: XSelect CSH Fluoro Phenyl 30*150 mm, 5 μm; Mobile Phase A: Water (0.1% FA), Mobile Phase B: MeCN; Flow rate: 60 mL/min; Gradient (B %): 48% B to 65% B in 30 min; Wave Length: 254 nm/220 nm; RT1 (min): 12.82, RT2 (min): 18.6) to afford 3-((S)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (Compound 10a) (1^{st} fraction, 10 mg, 10% yield, RT1 (min): 12.82) and 3-((R)-1-((R)-1-((1r,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (Compound 10b) (2^{nd} fraction, 59.9 mg, 61% yield, RT2 (min): 18.6) as a white solid.

| Compound# | Characterization Data |
|---|---|
| 10a | LCMS (ESI) [M − H]⁻: 623.5 |
| | ¹H NMR (400 MHz, Methanol-d4) δ 7.62 (s, 1H), 7.57 (d, J = 7.9 Hz, 1H), 7.49 (d, J = 8.4 Hz, |
| | 2H), 7.40 (d, J = 8.0 Hz, 1H), 7.01 (d, J = 8.4 Hz, 2H), 6.88 (s, 2H), 3.68-3.57 (m, 3H), 3.07 |
| | (dd, J = 10.8, 5.1 Hz, 1H), 3.04-2.94 (m, 1H), 2.89-2.79 (m, 1H), 2.61 (t, J = 6.9 Hz, 2H), |
| | 2.44-2.34 (m, 2H), 2.31-2.21 (m, 4H), 2.05-1.98 (m, 1H), 1.95 (s, 6H), 1.80-1.74 (m, |
| | 1H), 1.64-1.51 (m, 4H), 1.45-1.38 (m, 1H), 1.30-1.23 (m, 1H), 1.13-1.05 (m, 1H), 0.90 |
| | (s, 9H) |
| 10b | LCMS (ESI) [M − H]⁻: 623.5 |
| | ¹H NMR (400 MHz, Methanol-d4) δ 7.66 (d, J = 8.5 Hz, 1H), 7.58 (s, 1H), 7.53 (d, J = 7.8 Hz, |
| | 1H), 7.22 (d, J = 8.4 Hz, 2H), 6.91 (d, J = 8.4 Hz, 2H), 6.85 (s, 2H), 3.69-3.63 (m, 1H), 3.57 |
| | (t, J = 6.9 Hz, 2H), 3.25 (dd, J = 11.6, 4.7 Hz, 1H), 3.08-2.98 (m, 1H), 2.89-2.78 (m, 1H), |
| | 2.73-2.64 (m, 1H), 2.59 (t, J = 6.9 Hz, 2H), 2.41-2.32 (m, 1H), 2.30-2.15 (m, 4H), 2.15- |
| | 2.05 (m, 1H), 1.90 (s, 6H), 1.79-1.50 (m, 6H), 1.32-1.26 (m, 1H), 1.20-1.08 (m, 1H), 0.95 |
| | (s, 9H) |

### Example 11: 3-((S)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid and 3-((R)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

### Step 1: methyl 3-(1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate

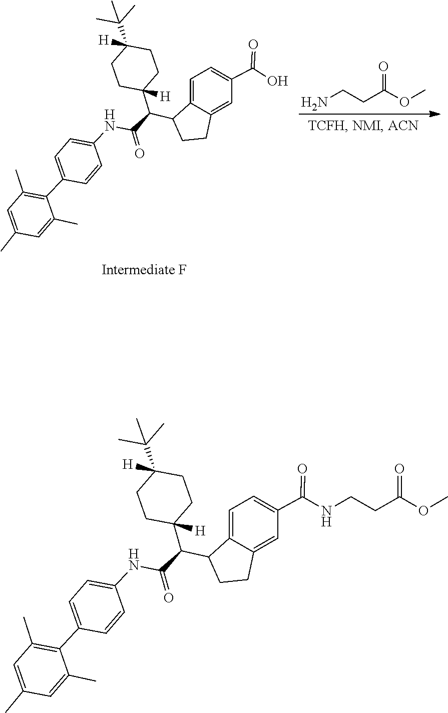

A solution of 1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate F, 120 mg, 0.15 mmol), methyl 3-aminopropanoate (57.4 mg, 0.35 mmol), TCFH (120.3 mg, 0.35 mmol) and NMI (36 mg, 0.35 mmol) in MeCN (3 mL) was stirred at room temperature for 1 hour. Upon completion, the resulting mixture was concentrated under reduced pressure. The crude residue was purified by silica gel column chromatography, eluted with PE/EA (1/1) to afford methyl 3-(1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate (120 mg, 84% yield) as a white solid. LCMS (ESI) [M+H]⁺: 637.4

### Step 2: 3-(1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

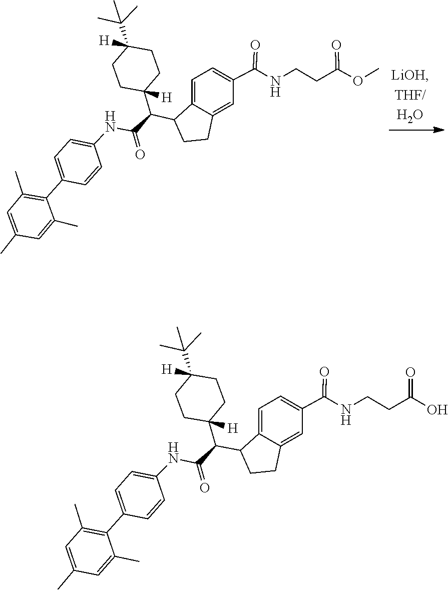

A solution of methyl 3-(1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate (50 mg, 0.07 mmol) and LiOH monohydrate (10 mg, 0.14 mmol) in H₂O (1 mL) and THF (1 mL) was stirred at room temperature for 1 hour. Upon completion, the mixture was acidified to pH=2 with HCl (1 M in water) and then extracted with EtOAc (3×20 mL). The combined organic layers were washed with water (20 mL), dried over anhydrous Na₂SO₄, filtered and concentrated under reduced pressure. The crude residue was purified by reversed-phase flash chromatography (conditions: column, C18 silica gel; mobile phase A: water (0.1% FA), Mobile phase B: MeCN; Gradient (B %): 10% to 50% in 10 min; detector, UV 254 nm) to afford 3-(1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (40 mg, 72% yield) as a white solid. LCMS (ESI) [M+H]⁺: 623.4

### Step 3: 3-((S)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid and 3-((R)-1-((S)-1-((1s,4R)-4-(tert-butyl)cyclohexyl)-2-oxo-2-((2′,4′,6′-trimethyl-[1,1′-biphenyl]-A-yl)amino)ethyl)-2,3-dihydro-1H-indene-5-carboxamido)propanoic acid

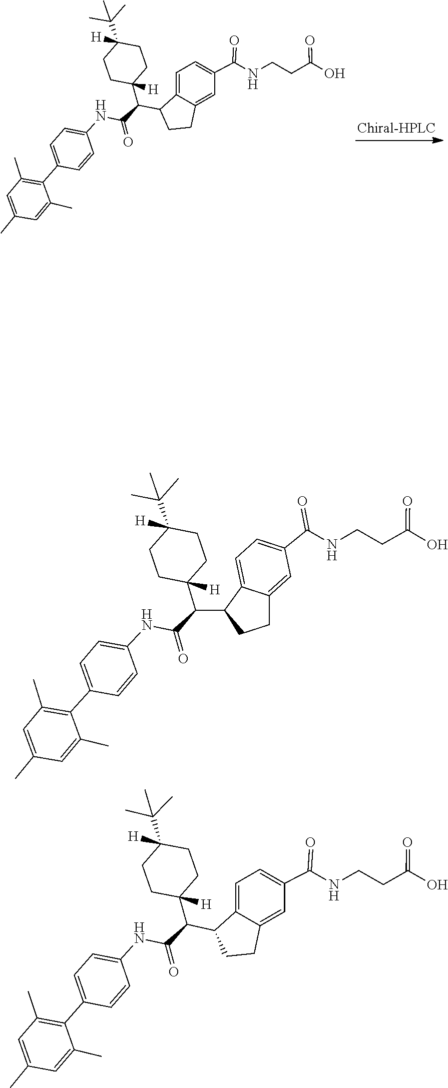 [SMILES: Cc1cc(C)c(-c2ccc(NC(=O)[C@H](C3CCc4cc(C(=O)NCCC(=O)O)ccc43)[C@H]3CC[C@@H](C(C)(C)C)CC3)cc2)c(C)c1 ; Cc1cc(C)c(-c2ccc(NC(=O)[C@@H]([C@H]3CC[C@@H](C(C)(C)C)CC3)[C@@H]3CCc4cc(C(=O)NCCC(=O)O)ccc43)cc2)c(C)c1 ; Cc1cc(C)c(-c2ccc(NC(=O)[C@@H]([C@H]3CC[C@@H](C(C)(C)C)CC3)[C@H]3CCc4cc(C(=O)NCCC(=O)O)ccc43)cc2)c(C)c1]

3-(1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (40 mg, 0.06 mmol) was separated by prep-Chiral HPLC (conditions: Column: CHIRALPAK ID, 3*25 cm, 5 μm; Mobile Phase A: Hex (0.1% FA), Mobile Phase B: EtOH; Flow rate: 40 mL/min; Gradient (B %): isocratic 10% B; Wave Length: 200/248 nm; RT1 (min): 10.6; RT2 (min): 19.9) to afford 3-((S)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (Compound 11a) (1^{st} fraction, 20.8 mg, 52% yield) and 3-((R)-1-((S)-1-((1s,4R)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (Compound 11b) (2^{nd} fraction, 12.5 mg, 42% yield).

| Compound# | Characterization Data |
|---|---|
| 11a | LCMS (ESI) [M − H]⁻: 623.5 |
| | ¹H NMR (400 MHz, Methanol-d4) δ 7.62 (d, J = 7.1 Hz, 2H), 7.44 (d, J = 8.3 Hz, 1H), 7.34 (d, |
| | J = 8.5 Hz, 2H), 6.95 (dd, J = 8.4, 1.9 Hz, 2H), 6.86 (s, 2H), 3.71 (q, J = 6.4 Hz, 1H), 3.58 (t, J = |
| | 6.9 Hz, 2H), 3.13-3.01 (m, 1H), 2.91-2.78 (m, 1H), 2.67-2.48 (m, 4H), 2.30-2.18 (m, |
| | 4H), 2.08-1.86 (m, 10H), 1.82-1.74 (m, 1H), 1.30-1.22 (m, 1H), 1.16-1.02 (m, 4H), 0.88 |
| | (s, 9H) |
| 11b | LCMS (ESI) [M − H]⁻: 623.5 |
| | ¹H NMR (400 MHz, Methanol-d4) δ 7.65 (s, 1H), 7.61-7.54 (m, 2H), 7.54-7.48 (m, 1H), |
| | 7.28 (d, J = 7.9 Hz, 1H), 7.05 (dd, J = 8.4, 1.9 Hz, 2H), 6.89 (s, 2H), 3.71 (q, J = 7.7 Hz, 1H), |
| | 3.58 (t, J = 6.9 Hz, 2H), 3.05-2.96 (m, 1H), 2.93-2.84 (m, 1H), 2.59 (t, J = 6.7 Hz, 2H), |
| | 2.53-2.45 (m, 1H), 2.36-2.25 (m, 4H), 2.10-1.96 (m, 8H), 1.92-1.80 (m, 4H), 1.43- |
| | 1.33 (m, 1H), 1.24-0.97 (m, 4H), 0.86 (s, 9H) |

### Example 12: 3-((S)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid and 3-((R)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

### Step 1: methyl 3-(1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate

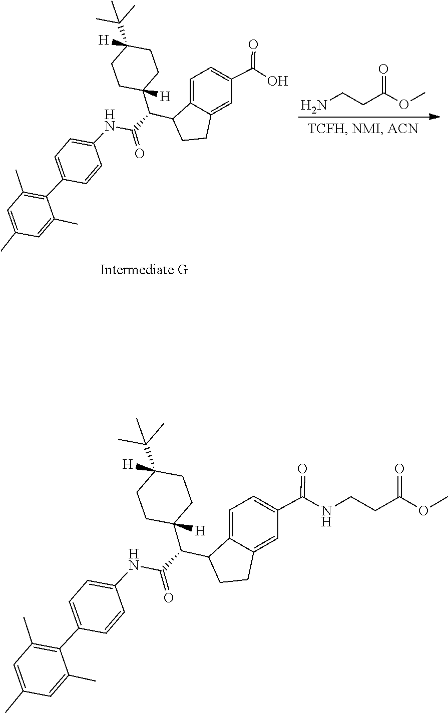

A mixture of 1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxylic acid (Intermediate G, 200 mg, 0.36 mmol), methyl 3-aminopropanoate (38 mg, 0.36 mmol), TCFH (154 mg, 0.54 mmol) and NMI (90 mg, 0.543 mmol) in MeCN (5 mL) was stirred at room temperature for 3 hours. Upon completion, the resulting mixture was concentrated under reduced pressure. The crude residue was purified by silica gel column chromatography, eluted with PE:EA (1:1) to afford methyl 3-(1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate (160 mg, 69% yield) as a yellow oil. LCMS (ESI) [M+H]⁺: 637.4

### Step 2: 3-(1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

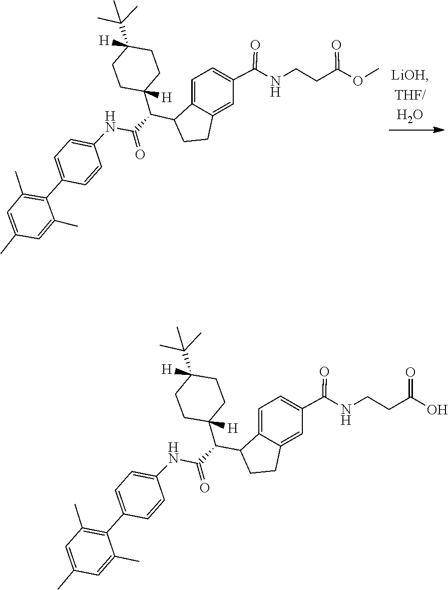

A solution of methyl 3-(1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoate (160 mg, 0.25 mmol) and LiOH monohydrate (58 mg, 1 mmol) in water (1 mL) and THF (1 mL) was stirred at room temperature for 1 hour. Upon completion, the mixture was acidified to pH=2 with HCl (1 M in water) and then extracted with EtOAc (3×20 mL). The combined organic layers were washed with water (20 mL), dried over anhydrous Na₂SO₄, filtered and concentrated under reduced pressure. The crude residue was purified by reversed-phase flash chromatography (conditions: column, C18 silica gel; mobile phase A: water (0.1% FA), mobile phase B: MeCN; Gradient (B %): 10% to 50% in 10 min; detector, UV 254 nm) to afford 3-(1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (85 mg, 54% yield) as a white solid. LCMS (ESI) [M+H]⁺: 623.4

### Step 3: 3-((S)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid and 3-((R)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid

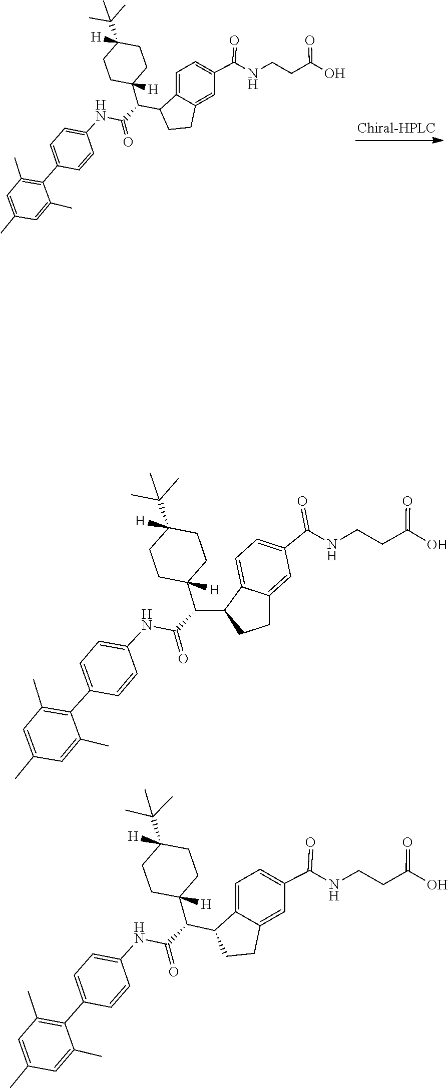 [SMILES: Cc1cc(C)c(-c2ccc(NC(=O)[C@@H](C3CCc4cc(C(=O)NCCC(=O)O)ccc43)[C@H]3CC[C@@H](C(C)(C)C)CC3)cc2)c(C)c1 ; Cc1cc(C)c(-c2ccc(NC(=O)[C@H]([C@H]3CC[C@@H](C(C)(C)C)CC3)[C@@H]3CCc4cc(C(=O)NCCC(=O)O)ccc43)cc2)c(C)c1 ; Cc1cc(C)c(-c2ccc(NC(=O)[C@H]([C@H]3CC[C@@H](C(C)(C)C)CC3)[C@H]3CCc4cc(C(=O)NCCC(=O)O)ccc43)cc2)c(C)c1]

3-(1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (85 mg, 0.10 mmol) was separated by prep-Chiral HPLC (conditions: Column: CHIRAL ART Cellulose-SC, 3*25 cm, 5 μm; Mobile Phase A: MtBE (0.5% 2M NH₃-MeOH), Mobile Phase B: MeOH; Flow rate: 20 mL/min; Gradient: isocratic 30% B; Wave Length: 212/250 nm; RT1 (min): 9.5; RT2 (min): 12) to afford 3-((S)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (Compound 12a) (1^{st} fraction, 19.6 mg, 23% yield) and 3-((R)-1-((R)-1-((1s,4S)-4-(tert-butyl)cyclohexyl)-2-oxo-2-((2′,4′,6′-trimethyl-[1,1′-biphenyl]-4-yl)amino)ethyl)-2,3-dihydro-1H-indene-5-carboxamido)propanoic acid (Compound 12b) (2^{nd} fraction, 16.3 mg, 19% yield) as a white solid.

| Compound# | Characterization Data |
|---|---|
| 12a | LCMS (ESI) [M − H]⁻: 623.5 |
| | ¹H NMR (400 MHz, Methanol-d4) δ 7.62 (d, J = 7.3 Hz, 2H), 7.44 (d, J = 8.2 Hz, 1H), 7.34 |
| | (d, J = 8.5 Hz, 2H), 6.95 (d, J = 8.4 Hz, 2H), 6.86 (s, 2H), 3.71 (q, J = 6.0 Hz, 1H), 3.58 (t, J = |
| | 6.9 Hz, 2H), 3.13-3.02 (m, 1H), 2.90-2.80 (m, 1H), 2.65-2.56 (m, 3H), 2.56-2.44 (m, |
| | 1H), 2.30-2.16 (m, 4H), 2.10-2.04 (m, 1H), 2.03-1.95 (m, 1H), 1.95-1.85 (m, 8H), 1.84- |
| | 1.76 (m, 1H), 1.28-1.19 (m, 1H), 1.18-1.01 (m, 4H), 0.88 (s, 9H) |
| 12b | LCMS (ESI) [M − H]⁻: 623.5 |
| | ¹H NMR (400 MHz, Methanol-d4) δ 7.65 (s, 1H), 7.57 (d, J = 8.6 Hz, 2H), 7.51 (d, J = 6.8 |
| | Hz, 1H), 7.28 (d, J = 8.0 Hz, 1H), 7.05 (d, J = 6.9 Hz, 2H), 6.89 (s, 2H), 3.71 (q, J = 7.3 Hz, |
| | 1H), 3.58 (t, J = 6.9 Hz, 2H), 3.06-2.97 (m, 1H), 2.93-2.83 (m, 1H), 2.60 (t, J = 6.9 Hz, |
| | 2H), 2.49 (dd, J = 9.5, 5.7 Hz, 1H), 2.38-2.25 (m, 4H), 2.11-2.03 (m, 1H), 1.98 (s, 7H), |
| | 1.93-1.76 (m, 4H), 1.43-1.36 (m, 1H), 1.19-1.00 (m, 4H), 0.86 (s, 9H) |

### Example 13: 2-((R)-1-((R)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid, 2-((S)-1-((S)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid, 2-((S)-1-((R)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((S)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

### Step 1: methyl 2-(4′-(tert-butyl)-[1,1′-biphenyl]-4-yl)acetate

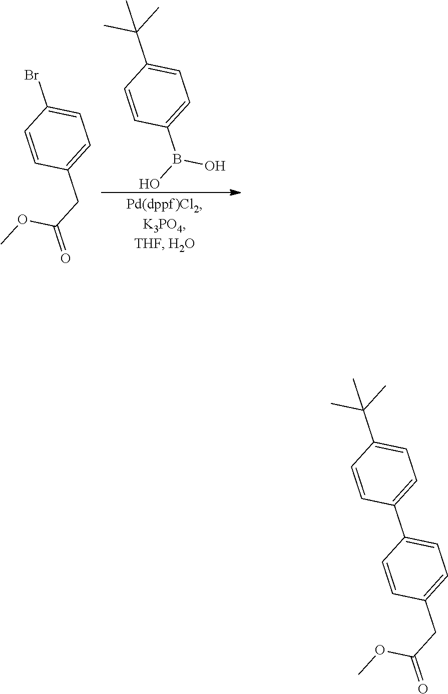 [SMILES: COC(=O)Cc1ccc(Br)cc1 ; CC(C)(C)c1ccc(B(O)O)cc1 ; COC(=O)Cc1ccc(-c2ccc(C(C)(C)C)cc2)cc1]

A solution of methyl 2-(4-bromophenyl)acetate (2.0 g, 8.73 mmol), (4-(tert-butyl)phenyl)boronic acid (3.11 g, 17.46 mmol), Pd(dppf)Cl₂ (0.64 g, 0.873 mmol) and potassium phosphate (5.56 g, 26.19 mmol) in THF (20 mL) and H₂O (2 mL) was heated at 80° C. for 2 hours under nitrogen atmosphere. Upon completion, the resulting mixture was concentrated under reduced pressure. The crude residue was purified by silica gel column chromatography, eluted with PE/EA (4:1) to afford methyl 2-(4′-(tert-butyl)-[1,1′-biphenyl]-4-yl)acetate (2.3 g, 93% yield) as a yellow solid. LCMS (ESI) [M+H]⁺: 283.3

### Step 2: 2-(4′-(tert-butyl)-[1,1′-biphenyl]-4-yl)acetic acid

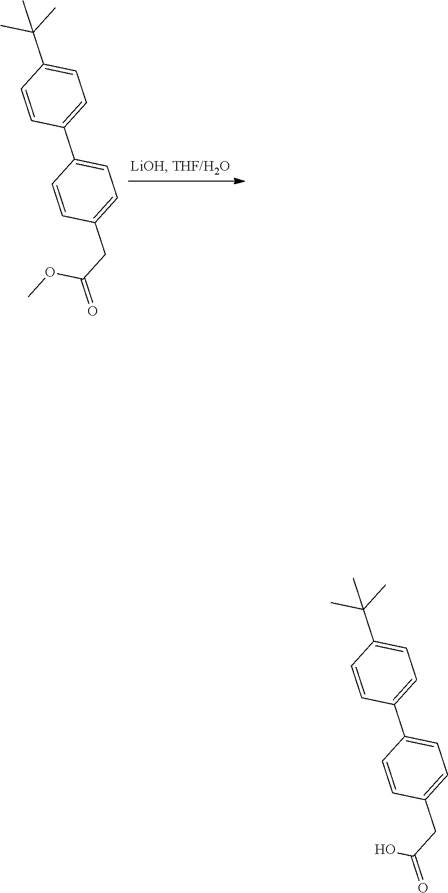 [SMILES: COC(=O)Cc1ccc(-c2ccc(C(C)(C)C)cc2)cc1 ; CC(C)(C)c1ccc(-c2ccc(CC(=O)O)cc2)cc1]

A solution of methyl 2-(4′-(tert-butyl)-[1,1′-biphenyl]-4-yl)acetate (2.3 g, 8.15 mmol) and LiOH monohydrate (0.59 g, 24.44 mmol) in THF (15 mL) and water (15 mL) was stirred at room temperature for 2 hours. Upon completion, the mixture was acidified to pH=1 with conc. HCl. The resulting mixture was extracted with EtOAc (3×20 mL). The combined organic layers were washed with brine (10 mL), dried over anhydrous Na₂SO₄, filtered and concentrated under reduced pressure. The crude residue was purified by reversed-phase flash chromatography (conditions: column, C18 silica gel; mobile phase A: Water (0.1% FA), mobile phase B: MeCN; Gradient (B %): 10% to 90% in 25 min; detector, UV 254 nm) to afford 2-(4′-(tert-butyl)-[1,1′-biphenyl]-4-yl)acetic acid (1.5 g, 68% yield) as a white solid. LCMS (ESI) [M−H]—: 267.2

### Step 3: 2-(5-bromo-1-hydroxy-2,3-dihydro-1H-inden-1-yl)-2-(4′-(tert-butyl)-[1,1′-biphenyl]-4-yl)acetic acid

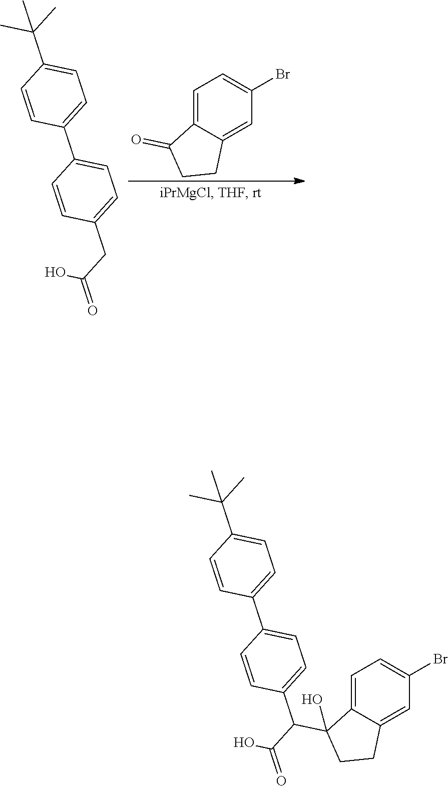

To a solution of 2-(4′-(tert-butyl)-[1,1′-biphenyl]-4-yl)acetic acid (1.4 g, 5.22 mmol) in THF (10 mL) was slowly added the solution of isopropylmagnesium chloride (10.4 mL, 10.4 mmol, 1 M in THF) at room temperature under nitrogen atmosphere. The resulting suspension was heated at 40° C. After 1 hour, the reaction was cooled to room temperature and 5-bromo-2,3-dihydro-1H-inden-1-one (2.20 g, 10.4 mmol) was added. The resulting solution was heated at 40° C. for 1 hour. Upon completion, the reaction mixture cooled to room temperature and was acidified to pH=2 with conc. HCl. The resulting mixture was extracted with EtOAc (3×20 mL). The combined organic layers were washed with brine (20 mL), dried over anhydrous Na₂SO₄, filtered and concentrated under reduced pressure. The crude residue was purified by reversed-phase flash chromatography (column, C18 silica gel; mobile phase A: Water (0.1% FA), mobile phase B: MeCN; Gradient (B %): 0% to 100% in 25 min; detector, UV 254 nm) to give 2-(5-bromo-1-hydroxy-2,3-dihydro-1H-inden-1-yl)-2-(4′-(tert-butyl)-[1,1′-biphenyl]-4-yl)acetic acid (920 mg, 36% yield) as a white solid. LCMS (ESI) [M+H]⁺: 479.1/481.1

### Step 4: 2-(6-bromo-1H-inden-3-yl)-2-(4′-(tert-butyl)-[1,1′-biphenyl]-4-yl)acetic acid

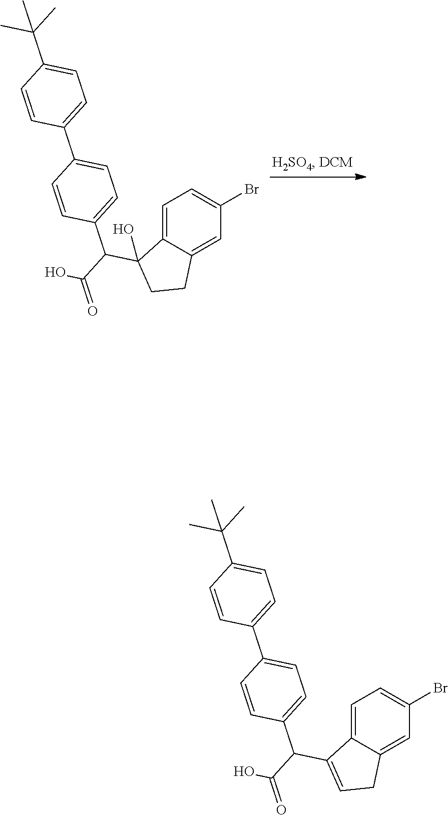 [SMILES: CC(C)(C)c1ccc(-c2ccc(C(C(=O)O)C3(O)CCc4cc(Br)ccc43)cc2)cc1 ; CC(C)(C)c1ccc(-c2ccc(C(C(=O)O)C3=CCc4cc(Br)ccc43)cc2)cc1]

2-(5-bromo-1-hydroxy-2,3-dihydro-1H-inden-1-yl)-2-(4′-(tert-butyl)-[1,1′-biphenyl]-4-yl)acetic acid (850 mg, 1.77 mmol) was dissolved in DCM (5.0 mL) and H₂SO₄ (0.5 mL) was added dropwise at room temperature. After 1 hour, the reaction was poured into ice water. The resulting white precipitate was collected by filtration, washed with water, PE and dried under reduced pressure to afford 2-(6-bromo-1H-inden-3-yl)-2-(4′-(tert-butyl)-[1,1′-biphenyl]-4-yl)acetic acid (600 mg, 73% yield) as a white solid, which was used in the next step without further purification. LCMS (ESI) [M+H]⁺: 461.2/463.2

### Step 5: 2-(6-bromo-1H-inden-3-yl)-2-(4′-(tert-butyl)-[1,1′-biphenyl]-4-yl)-N-(2′,4′,6′-trimethyl-[1,1′-biphenyl]-4-yl)acetamide

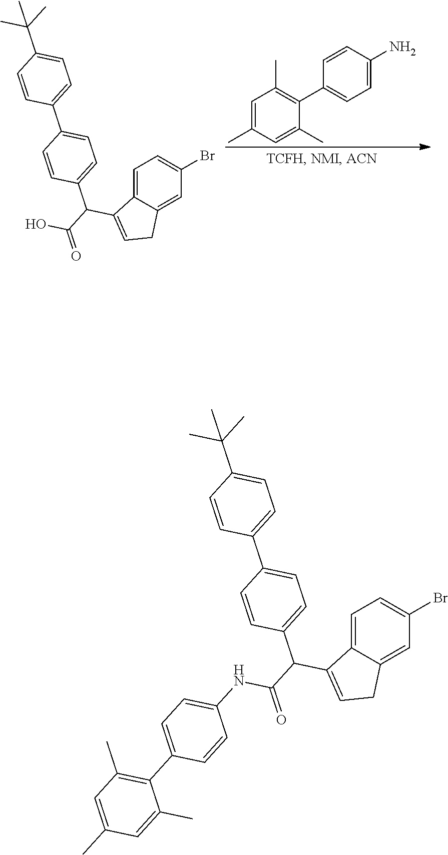

A solution of 2-(6-bromo-1H-inden-3-yl)-2-(4′-(tert-butyl)-[1,1′-biphenyl]-4-yl)acetic acid (580 mg, 1.26 mmol), 2′,4′,6′-trimethyl-[1,1′-biphenyl]-4-amine (531 mg, 2.51 mmol) and NMI (206 mg, 2.51 mmol) in MeCN (6.0 mL) was stirred at 0° C. for 15 minutes. Then, TCFH (705 mg, 2.51 mmol) was added in portions at 0° C. The resulting mixture was warmed to room temperature and was allowed to stir for 1 hour. Upon completion, the resulting mixture was concentrated under reduced pressure. The crude residue was purified by silica gel column chromatography, eluted with PE/EA (5/1) to afford 2-(6-bromo-1H-inden-3-yl)-2-(4′-(tert-butyl)-[1,1′-biphenyl]-4-yl)-N-(2′,4′,6′-trimethyl-[1,1′-biphenyl]-4-yl)acetamide (650 mg, 78% yield) as a yellow solid. LCMS (ESI) [M+H]⁺: 654.2/656.2

### Step 6: 3-(1-(4′-(tert-butyl)-[1,1′-biphenyl]-4-yl)-2-oxo-2-((2′,4′,6′-trimethyl-[1,1′-biphenyl]-4-yl)amino)ethyl)-1H-indene-6-carboxylic acid

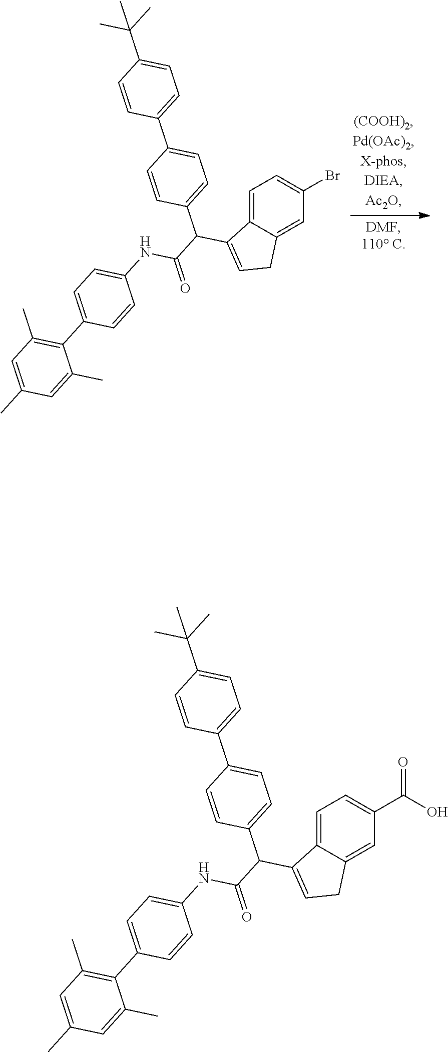 [SMILES: Cc1cc(C)c(-c2ccc(NC(=O)C(C3=CCc4cc(Br)ccc43)c3ccc(-c4ccc(C(C)(C)C)cc4)cc3)cc2)c(C)c1 ; Cc1cc(C)c(-c2ccc(NC(=O)C(C3=CCc4cc(C(=O)O)ccc43)c3ccc(-c4ccc(C(C)(C)C)cc4)cc3)cc2)c(C)c1]

A solution of 2-(6-bromo-1H-inden-3-yl)-2-(4′-(tert-butyl)-[1,1′-biphenyl]-4-yl)-N—(2′,4′,6′-trimethyl-[1,1′-biphenyl]-4-yl)acetamide (630 mg, 0.962 mmol), Pd(OAc)₂ (21.6 mg, 0.096 mmol), DIEA (249 mg, 1.92 mmol), 4,5-bis(diphenylphosphino)-9,9-dimethylxanthene (55.7 mg, 0.096 mmol), acetic anhydride (196 mg, 1.92 mmol) and oxalic acid anhydrous (173 mg, 1.92 mmol) in DMF (5.0 mL) was heated at 110° C. for 1 hour under nitrogen atmosphere. Upon completion, the reaction mixture was concentrated under reduced pressure. The crude residue was purified by reversed-phase flash chromatography (conditions: column, C18 silica gel; mobile phase A: Water (0.1% FA), mobile phase B: MeCN; Gradient (B %): 20% to 100% in 10 min; detector, UV 254 nm) to afford 3-(1-(4′-(tert-butyl)-[1,1′-biphenyl]-4-yl)-2-oxo-2-((2′,4′,6′-trimethyl-[1,1′-biphenyl]-4-yl)amino)ethyl)-1H-indene-6-carboxylic acid (350 mg, 58% yield) as a yellow solid. LCMS (ESI) [M−H]—: 618.3

### Step 7: 1-(1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxylic acid

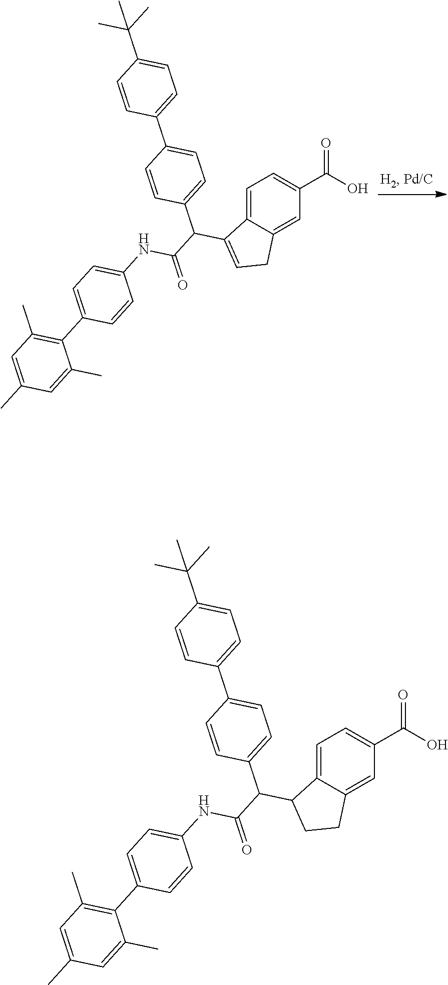 [SMILES: Cc1cc(C)c(-c2ccc(NC(=O)C(C3=CCc4cc(C(=O)O)ccc43)c3ccc(-c4ccc(C(C)(C)C)cc4)cc3)cc2)c(C)c1 ; Cc1cc(C)c(-c2ccc(NC(=O)C(c3ccc(-c4ccc(C(C)(C)C)cc4)cc3)C3CCc4cc(C(=O)O)ccc43)cc2)c(C)c1]

A solution of 3-(1-(4′-(tert-butyl)-[1,1′-biphenyl]-4-yl)-2-oxo-2-((2′,4′,6′-trimethyl-[1,1′-biphenyl]-4-yl)amino)ethyl)-1H-indene-6-carboxylic acid (345 mg, 0.56 mmol) and Pd/C (296 mg, 0.28 mmol, 10 wt % Pd on carbon) in THF (5.0 mL) was stirred at room temperature for 1 hour under hydrogen atmosphere. The resulting mixture was filtered, the filter cake was washed with EtOAc (3×8 mL). The filtrate was concentrated under reduced pressure to give crude 1-(1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxylic acid (325 mg, 93% yield) as a white solid which was used in the next step without further purification. LCMS (ESI) [M−H]—: 622.4.

### Step 8: 2-(1-(1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

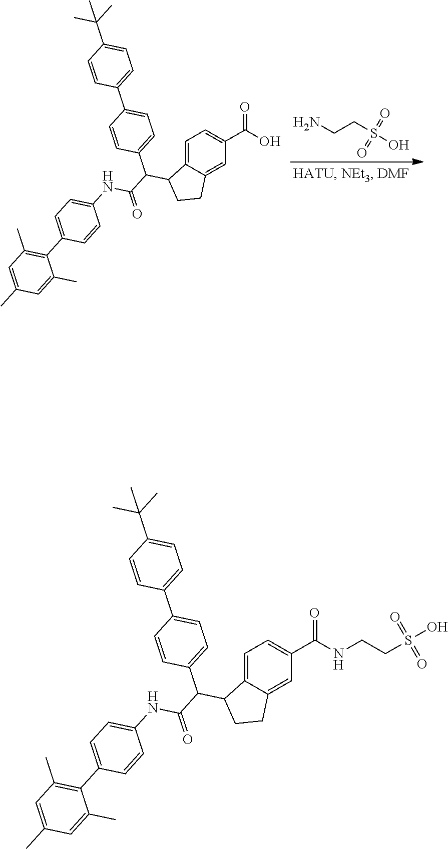 [SMILES: Cc1cc(C)c(-c2ccc(NC(=O)C(c3ccc(-c4ccc(C(C)(C)C)cc4)cc3)C3CCc4cc(C(=O)O)ccc43)cc2)c(C)c1 ; NCCS(=O)(=O)O ; Cc1cc(C)c(-c2ccc(NC(=O)C(c3ccc(-c4ccc(C(C)(C)C)cc4)cc3)C3CCc4cc(C(=O)NCCS(=O)(=O)O)ccc43)cc2)c(C)c1]

A solution of 1-(1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxylic acid (150 mg, 0.241 mmol), 2-aminoethanesulfonic acid (60.4 mg, 0.482 mmol) and TEA (48.8 mg, 0.482 mmol) in DMF (2.0 mL) was stirred at 0° C. After 15 minutes, HATU (183 mg, 0.482 mmol) was added in portions at 0° C. The resulting mixture was warmed to room temperature and stirred for an additional 1 hour. Upon completion, the reaction mixture was directly purified by reversed-phase flash chromatography (conditions: column, C18 silica gel; mobile phase A: Water (0.1% FA), mobile phase B: MeCN; Gradient (B %): 0% to 90% in 25 min; detector, UV 254 nm) to afford 2-(1-(1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (90 mg, 51% yield) as a white solid. LCMS (ESI) [M−H]—: 727.3

### Step 9: 2-((R)-1-((R)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid, 2-((S)-1-((S)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid, 2-((S)-1-((R)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid and 2-((R)-1-((S)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid

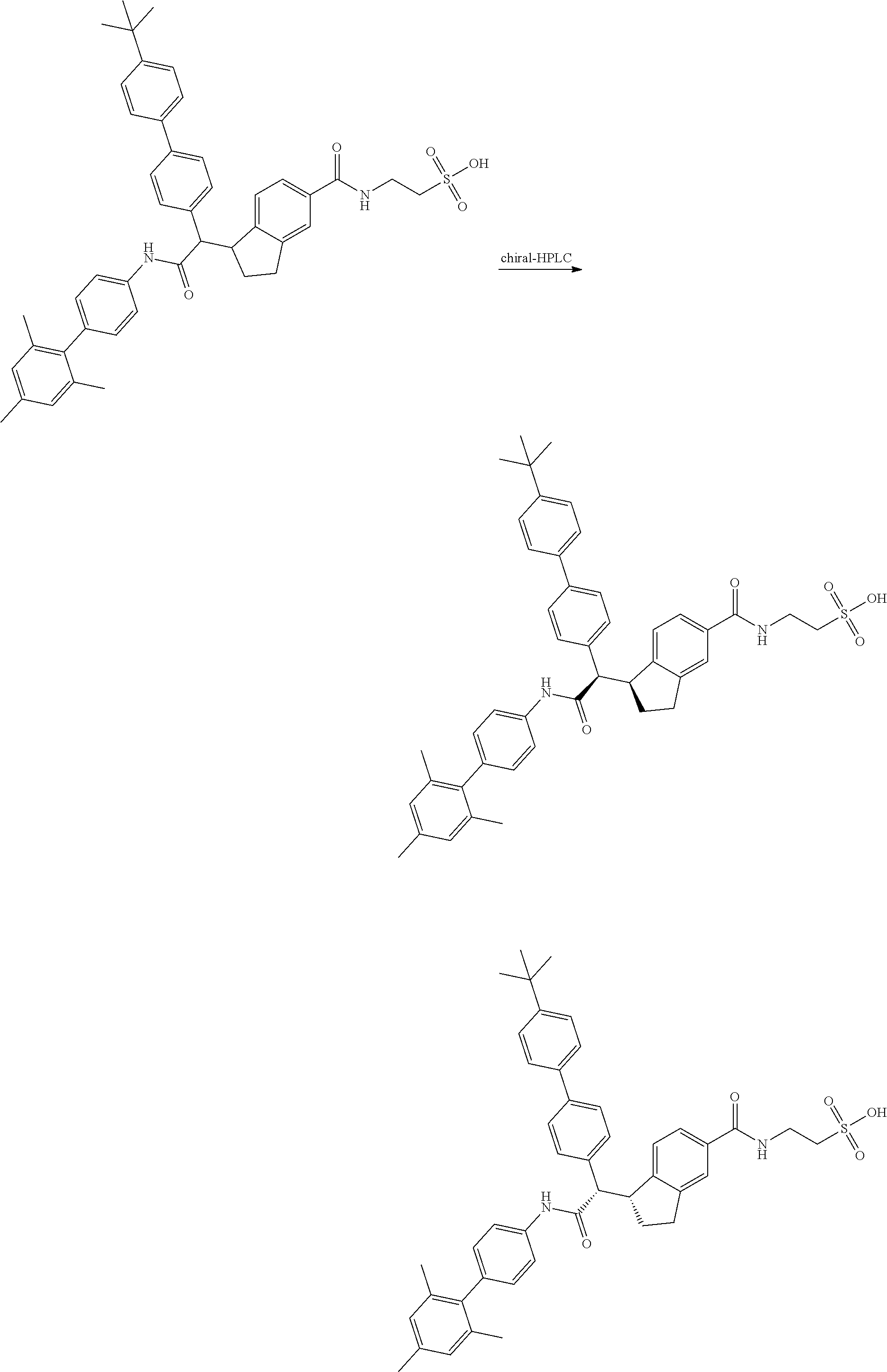 [SMILES: Cc1cc(C)c(-c2ccc(NC(=O)C(c3ccc(-c4ccc(C(C)(C)C)cc4)cc3)C3CCc4cc(C(=O)NCCS(=O)(=O)O)ccc43)cc2)c(C)c1 ; Cc1cc(C)c(-c2ccc(NC(=O)[C@@H](c3ccc(-c4ccc(C(C)(C)C)cc4)cc3)[C@@H]3CCc4cc(C(=O)NCCS(=O)(=O)O)ccc43)cc2)c(C)c1 ; Cc1cc(C)c(-c2ccc(NC(=O)[C@H](c3ccc(-c4ccc(C(C)(C)C)cc4)cc3)[C@H]3CCc4cc(C(=O)NCCS(=O)(=O)O)ccc43)cc2)c(C)c1]

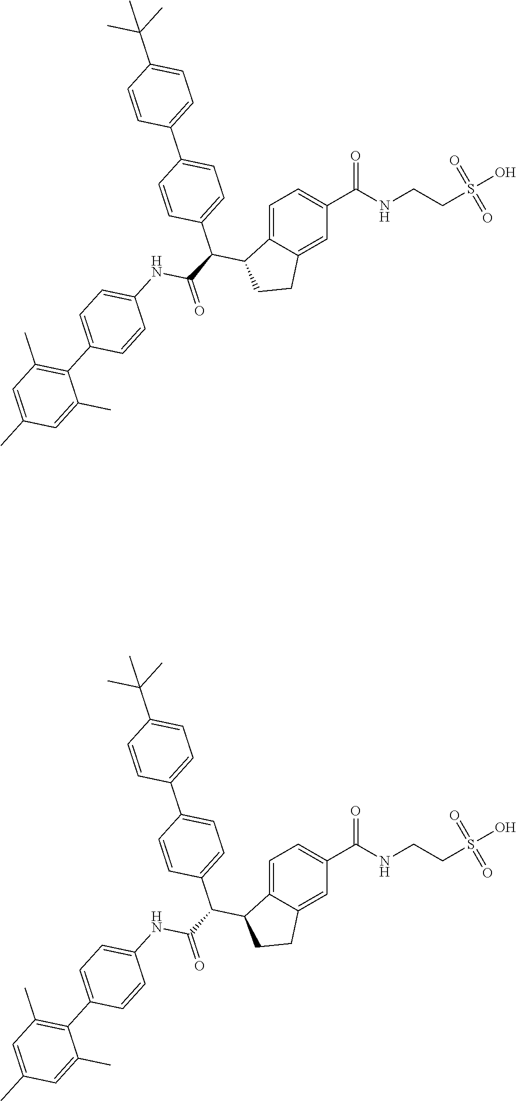 [SMILES: Cc1cc(C)c(-c2ccc(NC(=O)[C@@H](c3ccc(-c4ccc(C(C)(C)C)cc4)cc3)[C@H]3CCc4cc(C(=O)NCCS(=O)(=O)O)ccc43)cc2)c(C)c1 ; Cc1cc(C)c(-c2ccc(NC(=O)[C@H](c3ccc(-c4ccc(C(C)(C)C)cc4)cc3)[C@@H]3CCc4cc(C(=O)NCCS(=O)(=O)O)ccc43)cc2)c(C)c1]

2-(1-(1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (90 mg, 0.123 mmol) was separated by prep-Chiral HPLC (conditions: Column: CHIRALPAK ID, 3*25 cm, 5 μm; Mobile Phase A: Hex (0.1% DEA), Mobile Phase B: EtOH (0.1% TFA/0.1% DEA); Flow rate: 40 mL/min; Gradient (B %): isocratic 40% B; Wave Length: 220/254 nm; RT1 (min): 7.3; RT2 (min): 10.3; RT3 (min): 25.1) to afford 1^{st} fraction (30 mg, a mixture of 2 diastereomers, RT1 (min): 7.3), 2-((R)-1-((R)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 13a) (2^{nd} fraction, 6.7 mg, 7.4% yield, RT2 (min): 10.3) and 2-((S)-1-((S)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 13b) (3^{rd} fraction, 9.8 mg, 11% yield, RT3 (min): 25.1)

The 1^{st} fraction (30 mg, a mixture of 2 diastereomers) was further separated by prep Chiral-HPLC (conditions: Column: CHIRAL ART Cellulose-SZ, 3*25 cm, 5 m; Mobile Phase A: Hex (0.1% DEA), Mobile Phase B: EtOH (0.1% TFA/0.1% DEA); Flow rate: 40 mL/min; Gradient (B %): isocratic 30% B; Wave Length: 218/258 nm; RT1 (min): 9.90; RT2 (min): 14.12) to afford 2-((S)-1-((R)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 13c), (1^{st} fraction, 11.3 mg, 13% yield) and 2-((R)-1-((S)-1-(4′-(tert-butyl)-[1,1′-biphenyl]-4-yl)-2-oxo-2-((2′,4′,6′-trimethyl-[1,1′-biphenyl]-4-yl)amino)ethyl)-2,3-dihydro-1H-indene-5-carboxamido)ethane-1-sulfonic acid (Compound 13d) (2^{nd} fraction, 11.7 mg, 13% yield).

| Compound# | Characterization Data |
|---|---|
| 13a | LCMS (ESI) [M − H]⁻: 727.2 |
| | ¹H NMR (400 MHz, Methanol-d4) δ 7.71 (s, 1H), 7.68-7.51 (m, 9H), 7.47 (d, J = 8.4 |
| | Hz, 2H), 7.41 (d, J = 8.0 Hz, 1H), 7.03 (d, J = 8.3 Hz, 2H), 6.88 (s, 2H), 4.19-4.12 (m, |
| | 1H), 3.79 (t, J = 6.8 Hz, 2H), 3.69 (d, J = 11.0 Hz, 1H), 3.07 (t, J = 6.6 Hz, 2H), 3.03- |
| | 2.95 (m, 1H), 2.90-2.83 (m, 1H), 2.27 (s, 3H), 2.13-2.04 (m, 1H), 1.95 (s, 6H), 1.80- |
| | 1.73 (m, 1H), 1.35 (s, 9H) |
| 13b | LCMS (ESI) [M + H]⁺: 729.4 |
| | ¹H NMR (400 MHz, Methanol-d4) δ 7.71 (s, 1H), 7.69-7.51 (m, 9H), 7.47 (d, J = 8.4 |
| | Hz, 2H), 7.41 (d, J = 8.1 Hz, 1H), 7.03 (d, J = 8.5 Hz, 2H), 6.88 (s, 2H), 4.20-4.11 (m, |
| | 1H), 3.79 (t, J = 6.6 Hz, 2H), 3.69 (d, J = 11.2 Hz, 1H), 3.07 (t, J = 6.6 Hz, 2H), 3.03- |
| | 2.94 (m, 1H), 2.92-2.83 (m, 1H), 2.27 (s, 3H), 2.13-2.03 (m, 1H), 1.95 (s, 6H), 1.82- |
| | 1.72 (m, 1H), 1.35 (s, 9H) |
| 13c | LCMS (ESI) [M − H]⁻: 727.2 |
| | ¹H NMR (400 MHz, Methanol-d4) δ 7.67 (s, 1H), 7.65-7.56 (m, 6H), 7.49 (dd, J = 8.2, |
| | 5.2 Hz, 4H), 7.27 (d, J = 7.9 Hz, 1H), 7.04 (d, J = 8.2 Hz, 2H), 6.88 (s, 2H), 6.31 (d, J = |
| | 8.0 Hz, 1H), 4.10-4.02 (m, 1H), 3.76 (t, J = 6.6 Hz, 2H), 3.70 (d, J = 11.2 Hz, 1H), 3.17- |
| | 3.11 (m, 1H), 3.05 (t, J = 6.6 Hz, 2H), 3.01-2.93 (m, 1H), 2.53-2.40 (m, 1H), 2.27 |
| | (s, 3H), 2.13-2.06 (m, 1H), 1.95 (s, 6H), 1.36 (s, 9H) |
| 13d | LCMS (ESI) [M − H]⁻: 727.2 |
| | ¹H NMR (400 MHz, Methanol-d4) δ 7.67 (s, 1H), 7.66-7.56 (m, 6H), 7.49 (dd, J = 8.4, |
| | 5.2 Hz, 4H), 7.27 (d, J = 7.9 Hz, 1H), 7.04 (d, J = 8.5 Hz, 2H), 6.88 (s, 2H), 6.31 (d, J = |
| | 8.0 Hz, 1H), 4.10-4.02 (m, 1H), 3.76 (t, J = 6.7 Hz, 2H), 3.70 (d, J = 11.1 Hz, 1H), 3.19- |
| | 3.11 (m, 1H), 3.05 (t, J = 6.7 Hz, 2H), 3.01-2.92 (m, 1H), 2.53-2.42 (m, 1H), 2.27 |
| | (s, 3H), 2.14-2.05 (m, 1H), 1.95 (s, 6H), 1.36 (s, 9H) |

### Biological Examples

The utility of the compounds of the present invention and the salts of such compounds as medical agents in the treatment of the above described disease/conditions in mammals (e.g. humans, male or female) is demonstrated by the activity of the compounds of the present invention in one or more of the conventional assays and in vivo assays described and noted below. The in vivo assays (with appropriate modifications within the skill in the art) can be used to determine the activity of other agents as well as the compounds of the present invention. Thus, the protocols described below can also be used to demonstrate the utility of the combinations of the compounds of the present invention. The assays and models may also demonstrate particular other property advantages e.g., side effect profile; half-life. In addition, such assays provide a means whereby the activities of the compounds of the present invention and the salts of such compounds (or the other agents described herein) can be compared to each other and with the activities of other known compounds. The results of these comparisons are useful for determining dosage levels in mammals, including humans, for the treatment of such diseases.

Absorption, Distribution, Metabolism and Excretion (ADME) and pharmacokinetics (PK) of compounds and exemplary assays are discussed in the on-line publication by Thomas D. Y. Chung, David B. Terry and Layton H. Smith “In Vitro and In Vivo Assessment of ADME and PK Properties During Lead Selection and Lead Optimization-Guidelines, Benchmarks and Rules of Thumb-(https://www.ncbi.nlm.nih.gov/books/NBK326710/).

Exemplary in vitro and in vivo models of obesity are described in “A Review on in-vivo and in-vitro Models of Obesity and Obesity-Associated Co-Mmorbidities” Digbijoy Nath, Pervej Alom Barbhuiya, Saikat Sen, Manash Pratim Pathak; Endocr Metab Immune Disord Drug Targets (2024 Aug. 12).

Exemplary in vivo models of autoimmune disease/conditions are included in Autoimmun Rev. 2018 May; 17(5): 473-479. Exemplary in vitro assays of cancer are described in Front Bioeng Biotechnol 2016; 4:12 “In Vitro Tumore Models: Advantages, Disadvantages, Variables, and Selecting the Right Platform.” Exemplary in vivo animal models of cancer are described in Front Oncol, 2019; 8: 429 “Next-Generation in vivo Modeling of Human Cancers. Exemplary in vivo animal models of pain are described in Journal of Neuroscience Methods Volume 348, January 2021, 108997, “Animal models of pain: Diversity and benefits” and J. Pain. 2013 November: 14(11): 10.1016/j.jpain.2013.06.008 “An overview of animal models of pain: disease models and outcome measures”.

The following protocols may of course be varied by those skilled in the art.

hGIPR Antagonist Assay:

hGIPR/CHO-K1 cell line stably transfected to express human GIPR (gene of interest) were maintained at 37° C. with 5% CO₂ and grown in Ham's F12 media supplemented with 10% FBS, 1% penicillin/streptomycin, and 10 μg/mL of puromycin.

On the day of experiment, cells were harvested and counted using a Countess II cell counter; only cells with viability >90% were used for assays. Cells were diluted to 1E⁵ cells/mL with cAMP assay buffer (1×HBSS with Ca²⁺ and Mg²⁺, 20 mM HEPES, 0.1% BSA, and 500 μM IBMX). Compounds were transferred into 384-well assay plates in determined concentration response range, with constant percentage of DMSO below 0.5%, 10 μL of cells were then seeded into plates, and briefly centrifuged for 1 min at 600 rpm. Compounds were allowed to incubate with cells for 30 min at 25° C., after which assay buffer containing GIP at EC₈₀ was added to each well. Assay plate was once again briefly centrifuged for 1 min at 600 rpm, and then incubated for an additional 30 min at 25° C. Detection reagent was the LANCE Ultra cAMP kit from Revvity (TRF0264). 5 μL/well of Eu-cAMP tracer working solution and 5 μL/well of anti-cAMP working solution were added to each well and assay plate was centrifuged for 1 min at 600 rpm. Assay plate was allowed to incubate for 60 min at room temperature, and results were read with EnVision microplate reader ((λex=337 nm, λem=615 nm and 665 nm). The HTRF ratio was calculated from raw data (channel 1/channel 2×10,000), concentration response curve was plotted and IC₅₀ was calculated with 4 parameter nonlinear regression model.

hGCGR Antagonist Assay:

hGCGR/Flp in CHO-K1 cell line stably transfected to express human GCGR (gene of interest) were maintained at 37° C. with 5% CO₂ and grown in Ham's F12 media supplemented with 10% FBS, 1% penicillin/streptomycin, and 800 μg/mL of Hygromycin B.

On the day of experiment, cells were harvested and counted using a Countess II cell counter; only cells with viability >90% were used for assays. Cells were diluted to 2E⁵ cells/mL with cAMP assay buffer (1×HBSS with Ca²⁺ and Mg²+, 20 mM HEPES, 0.1% BSA, and 500 μM IBMX). Compounds were transferred into 384-well assay plates in determined concentration response range, with constant percentage of DMSO below 0.5%, 10 μL of cells were then seeded into plates, and briefly centrifuged for 1 min at 600 rpm. Compounds were allowed to incubate with cells for 30 min at 25° C., after which assay buffer containing glucagon at EC₈₀ was added to each well. Assay plate was once again briefly centrifuged for 1 min at 600 rpm, and then incubated for an additional 30 min at 25° C. Detection reagent was the LANCE Ultra cAMP kit from Revvity (TRF0264). 5 μL/well of Eu-cAMP tracer working solution and 5 μL/well of anti-cAMP working solution were added to each well and assay plate was centrifuged for 1 min at 600 rpm. Assay plate was allowed to incubate for 60 min at room temperature, and results were read with EnVision microplate reader ((λex=337 nm, λem=615 nm and 665 nm). The HTRF ratio was calculated from raw data (channel 1/channel 2×10,000), concentration response curve was plotted and IC₅₀ was calculated with 4 parameter nonlinear regression model. The results are shown in Table 2.

TABLE 2
IC₅₀ of tested compounds in hGIPR and hGCGR antagonist assays

| Compound # | hGIPR IC₅₀ (nM) | hGCGR IC₅₀ (nM) |
|---|---|---|
| 1a | 74 | 201 |
| 1b | >10000 | 9840 |
| 2a | 793 | 1170 |
| 2b | 5760 | >10000 |
| 3a | 8520 | >10000 |
| 3b | 2300 | 2020 |
| 4a | 6270 | >10000 |
| 4b | >10000 | >10000 |
| 4c | 6320 | 6220 |
| 4d | >10000 | >10000 |
| 9a | 5580 | 648 |
| 9b | >10000 | 9140 |
| 14a | 8830 | >10000 |
| 14b | 3130 | 8240 |
| 5a | >10000 | >10000 |
| 5b | >10000 | >10000 |
| 6a | 6860 | >10000 |
| 6b | 6570 | 6600 |
| 7a | 6550 | >10000 |
| 7b | 6970 | >10000 |
| 8a | 2290 | 6320 |
| 8b | 6950 | >10000 |
| 10a | 428 | 203 |
| 10b | 60 | 84 |
| 11a | 2350 | 562 |
| 11b | 8560 | >10000 |
| 12a | 41 | 156 |
| 12b | >10000 | 721 |
| 14c | 7860 | 5690 |
| 14d | 1470 | 6410 |

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A compound of Formula (I) or a pharmaceutically acceptable salt thereof: 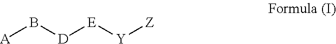
wherein
A is —C(O)OR² or —S(O)₂OR²;
R² is H or (C₁-C₆)alkyl;
B is (C₁-C₄)alkylene;
D is NH, CH₂, or is absent;
E is carbonyl, sulfonyl, NH, CH₂ or is absent;
Y is 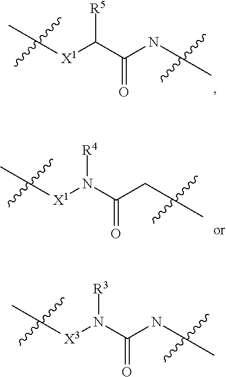
wherein
when Y is 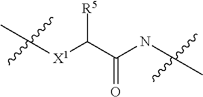
X¹ is a bicyclic ring comprising a 5-6 membered aryl ring, optionally having 0 to 3 heteroatoms selected independently from nitrogen, oxygen and sulfur, fused to a (C₅-C₇)cycloalkyl ring optionally containing 0 to 3 heteroatoms independently selected from nitrogen, oxygen and sulfur;
X¹ is optionally mono-, di- or tri-substituted independently with (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, difluoromethoxy or trifluoromethoxy;
R⁵ is (C₅-C₆)alkyl, phenyl, hetero(C₅-C₆)aryl, (C₃-C₇)cycloalkyl, (C₃-C₇)cycloalkenyl or hetero(C₃-C₇)cycloalkyl; said hetero(C₅-C₆)aryl and hetero(C₃-C₇)cycloalkyl each independently having 1 to 3 heteroatoms selected independently from nitrogen, oxygen and sulfur;
wherein said R⁵ is optionally mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, pentafluoropropanyl, mono-N— or di-N,N—(C₁-C₆)alkylaminocarbonyl, mono-N— or di-N,N—(C₁-C₆)alkylaminocarbonyloxy, (C₁-C₆)alkylcarbonyloxy, (C₁-C₆)alkylcarbonyl or (C₁-C₆)alkoxycarbonyl;
or R⁵ forms a 5-7 membered heteroaryl or heteroalkyl ring with nitrogen, optionally said heteroaryl or heteroalkyl ring each independently having one or two additional heteroatoms selected independently from nitrogen, sulfur and oxygen having the Formula X, wherein said 5-7 membered ring formed with nitrogen is optionally substituted with (C₁-C₄)alkyl, or is mono- or di-substituted independently with T¹; 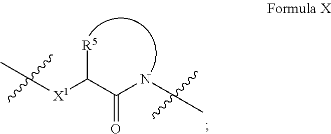
wherein T¹ is phenoxy, hetero(C₅-C₆)aryloxy, (C₃-C₇)cycloalkoxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl;
wherein said hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said T¹ is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; and
Z is Z¹; and Z¹ is phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryl or hetero(C₃-C₇)cycloalkyl each independently have 1 to 3 heteroatoms selected independently from oxygen, nitrogen and sulfur;
wherein said Zi is mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, (C₃-C₆)cycloalkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; or wherein said Z¹ is mono- or di-substituted independently with Q¹;
wherein Q¹ is phenoxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl;
wherein hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said Q¹ is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl;
or Z is Z¹ and Z¹ forms a 5-7 membered heteroaryl or heteroalkyl ring with R⁵, optionally said heteroaryl or heteroalkyl each independently having one or two additional heteroatoms selected independently from nitrogen, sulfur and oxygen having the Formula XI: 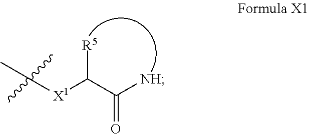
or
when Y is 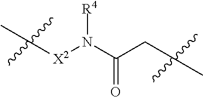
X² is a bicyclic ring comprising a 5-6 membered aryl ring optionally having 0 to 3 heteroatoms selected independently from nitrogen, oxygen and sulfur fused to a (C₅-C₇)cycloalkyl ring optionally containing 0 to 3 heteroatoms independently selected from nitrogen, oxygen and sulfur;
X² is optionally mono-, di- or tri-substituted with (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, difluoromethoxy or trifluoromethoxy;
R⁴ is (C₅-C₆)alkyl, phenyl, hetero(C₅-C₆)aryl, (C₃-C₇)cycloalkyl, (C₃-C₇)cycloalkenyl or hetero(C₃-C₇)cycloalkyl; said hetero(C₅-C₆)aryl and hetero(C₃-C₇)cycloalkyl each independently having 1 to 3 heteroatoms selected independently from nitrogen, oxygen and sulfur;
wherein said R⁴ is optionally mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, pentafluoropropanyl, mono-N— or di-N,N—(C₁-C₆)alkylaminocarbonyl, mono-N— or di-N,N—(C₁-C₆)alkylaminocarbonyloxy, (C₁-C₆)alkylcarbonyloxy, (C₁-C₆)alkylcarbonyl or (C₁-C₆)alkoxycarbonyl;
or R⁴ forms a 5-7 membered heteroaryl or heteroalkyl ring with nitrogen, said heteroaryl or heteroalkyl ring each optionally having one or two additional heteroatoms selected independently from nitrogen, sulfur and oxygen having the Formula X2; wherein said ring formed with nitrogen is optionally substituted with (C₁-C₄)alkyl; or wherein said R⁴ is mono- or di-substituted independently with T²; 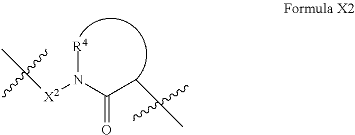
wherein T² is phenoxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said T² is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; and
Z is Z²; and Z² is phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryl or hetero(C₃-C₇)cycloalkyl each independently have 1 to 3 heteroatoms selected independently from oxygen, nitrogen and sulfur;
wherein said Z² is mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkoxy(C₁-C₆)alkyl, difluoromethyl, trifluoromethyl, trifluoroethyl, trifluoromethylthio, pentafluoroethanyl, or pentafluoropropanyl; or Z² is mono- or di-substituted independently with Q²
wherein Q² is phenoxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said Q² is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl;
or Z is Z²; and Z² forms a 5-7 membered heteroaryl or heteroalkyl ring with R⁴, optionally said heteroaryl or heteroalkyl each independently having one or two additional heteroatoms selected independently from nitrogen, sulfur and oxygen having the formula X3 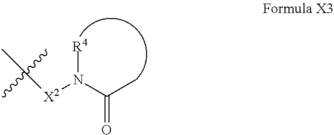
or
when Y is 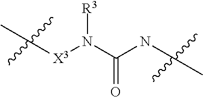
X³ is a bicyclic ring comprising a 5-6 membered aryl ring optionally having 0-3 heteroatoms selected independently from nitrogen, oxygen and sulfur fused to a (C₅-C₇)cycloalkyl ring optionally containing 0 to 3 heteroatoms independently selected from nitrogen, oxygen and sulfur;
X³ is optionally mono-, di- or tri-substituted with (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, difluoromethoxy or trifluoromethoxy;
R³ is (C₅-C₆)alkyl, phenyl, hetero(C₅-C₆)aryl, (C₃-C₇)cycloalkyl, (C₃-C₇)cycloalkenyl or hetero(C₃-C₇)cycloalkyl; said hetero(C₅-C₆)aryl and hetero(C₃-C₇)cycloalkyl each independently having 1 to 3 heteroatoms selected independently from nitrogen, oxygen and sulfur;
wherein said R³ is optionally mono-, di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, pentafluoropropanyl, mono-N— or di-N,N—(C₁-C₆)alkylaminocarbonyl, mono-N— or di-N,N—(C₁-C₆)alkylaminocarbonyloxy, (C₁-C₆)alkylcarbonyloxy, (C₁-C₆)alkylcarbonyl or (C₁-C₆)alkoxycarbonyl;
or R³ forms a 5-7 membered heteroaryl or heteroalkyl ring with nitrogens, said heteroaryl or heteroalkyl ring each optionally having one or two additional heteroatoms selected independently from nitrogen, sulfur and oxygen having the formula X4 wherein said ring formed with nitrogens is optionally substituted with (C₁-C₄)alkyl; or wherein said R³ is mono- or di-substituted independently with T³; 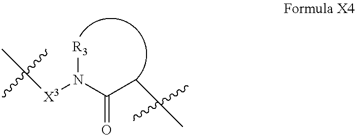
wherein T³ is phenyloxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said T³ is optionally mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl; and
Z is Z³; and Z³ is phenyl, hetero(C₅-C₆)aryl, (C₃-C₇)cycloalkyl, (C₃-C₇)cycloalkenyl or hetero(C₃-C₇)cycloalkyl; wherein said hetero(C₅-C₆)aryl or hetero(C₃-C₇)cycloalkyl each independently have 1 to 3 heteroatoms selected independently from oxygen, nitrogen and sulfur;
wherein said Z³ is mono-, di- or tri-substituted independently with cyano, cyanomethyl, nitro, amino, thio, mono-N— or di-N,N—(C₁-C₆)alkylamino, mono-N— or di-N,N—(C₁-C₆)alkylamino(C₁-C₆)alkyl, (C₃-C₆)cycloalkyl, (C₁-C₆)alkoxy(C₁-C₆)alkyl, difluoromethyl, trifluoroethyl, trifluoromethylthio, pentafluoroethanyl, or pentafluoropropanyl; or Z³ is mono- or di-substituted independently with Q³;
wherein Q³ is phenyloxy, hetero(C₅-C₆)aryloxy, (C₃-C₇)cycloalkyloxy, (C₃-C₇)cycloalkenyloxy, hetero(C₃-C₇)cycloalkyloxy, phenylthio, hetero(C₅-C₆)arylthio, (C₃-C₇)cycloalkylthio, (C₃-C₇)cycloalkenylthio, hetero(C₃-C₇)cycloalkylthio, phenyl, hetero(C₅-C₆)aryl, (C₃-C₇)cycloalkyl, (C₃-C₇)cycloalkenyl or hetero(C₃-C₇)cycloalkyl; wherein hetero(C₅-C₆)aryloxy, hetero(C₃-C₇)cycloalkyloxy, hetero(C₅-C₆)arylthio, hetero(C₃-C₇)cycloalkylthio, hetero(C₅-C₆)aryl or hetero(C₃-C₇)cycloalkyl each independently have 1 to 3 heteroatoms selected independently from oxygen, nitrogen and sulfur;
wherein said Q³ is mono- di- or tri-substituted independently with cyano, cyanomethyl, halo, nitro, amino, thio, mono-N— or di-N,N—(C₁-C₆)alkylamino, mono-N— or di-N,N—(C₁-C₆)alkylamino(C₁-C₆)alkyl, (C₁-C₆)alkyl, (C₁-C₆)alkoxy, (C₁-C₆)alkoxy(C₁-C₆)alkyl, difluoromethyl, trifluoromethyl, trifluoroethyl, trifluoromethylthio, fluoromethoxy, difluoromethoxy, trifluoromethoxy, pentafluoroethanyl, or pentafluoropropanyl;
or Z is Z³; and Z³ forms a 5-7 membered heteroaryl or heteroalkyl ring with R³, optionally said heteroaryl or heteroalkyl each independently having one or two additional heteroatoms selected independently from nitrogen, sulfur and oxygen having the formula X5 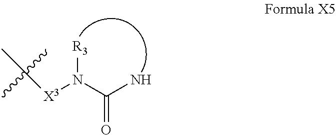
with the proviso that the compound of Formula (I) is not N—({1-[(trans-4-tert-butylcyclohexyl)({[4-(trifluoromethoxy)phenyl]amino}carbonyl]amino)-2,3-dihydro-1H-inden-5-yl}carbonyl)-B-alanine.

2. The compound of claim 1, wherein
A is —C(O)OH;
B is (C₁-C₄)alkylene;
D is NH;
E is carbonyl;
Y is 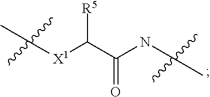
X¹ is 2,3-dihydro-1H-indenyl, indolinyl, tetrahydroquinolinyl or tetrahydronaphthalenyl;
wherein said X¹ is optionally mono-substituted with (C₁-C₆)alkyl or trifluoromethyl;
R⁵ is phenyl or (C₃-C₇)cycloalkyl; said phenyl or (C₃-C₇)cycloalkyl mono-substituted with (C₁-C₆)alkyl, phenyl or (C₃-C₇)cycloalkyl; and
Z¹ is phenyl mono-, di- or tri-substituted independently with (C₁-C₆)alkyl, (C₁-C₆)alkoxy, trifluoromethyl or trifluoromethylthio;
or
said Z¹ is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio.

3. The compound of claim 2, wherein R⁵ is phenyl or (C₅-C₈)cycloalkyl; said phenyl or C₅-C₈)cycloalkyl mono-substituted with (C₁-C₆)alkyl.

4. The compound of claim 2, wherein R⁵ is phenyl or (C₃-C₇)cycloalkyl; said phenyl or (C₃-C₇)cycloalkyl mono-substituted with phenyl or (C₃-C₇)cycloalkyl.

5. The compound of claim 2, wherein Z¹ is phenyl; said phenyl mono-, di- or tri-substituted independently with (C₁-C₆)alkyl, (C₁-C₆)alkoxy, trifluoromethyl or trifluoromethylthio,

6. The compound of claim 2, wherein Z¹ is phenyl, said phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio.

7. The compound of claim 2, wherein R⁵ is phenyl or (C₃-C₇)cycloalkyl; said phenyl or (C₃-C₇)cycloalkyl mono-substituted with phenyl or (C₃-C₇)cycloalkyl; and Z¹ is phenyl which is mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromeththio.

8. The compound of claim 1, wherein
A is —C(O)OH;
B is (C₁-C₄)alkylene;
D is NH;
E is carbonyl;
Y is 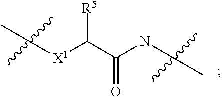
X¹ is 2,3-dihydro-1H-indenyl, indolinyl, tetrahydroquinolinyl or tetrahydronaphthalenyl;
X¹ is optionally mono-substituted with (C₁-C₆)alkyl or trifluoromethyl;
R⁵ forms a 5-7 membered heteroaryl or heteroalkyl ring with nitrogen, optionally said heteroaryl or heteroalkyl ring each independently having one or two additional heteroatoms selected independently from nitrogen, sulfur and oxygen having the formula X, wherein said ring formed with nitrogen is optionally substituted with (C₁-C₄)alkyl; 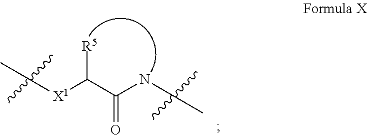
Z¹ is phenyl mono-, di- or tri-substituted independently with (C₁-C₆)alkyl, (C₁-C₆)alkoxy, trifluoromethyl or trifluoromethylthio;
or Z¹ is phenyl mono-substituted with (C₃-C₇)cycloalkyl, phenyl, phenoxy or benzyl wherein said (C₃-C₇)cycloalkyl, phenyl, phenoxy or benzyl are mono-, di- or tri-substituted independently with (C₁-C₆)alkyl, (C₁-C₆)alkoxy, trifluoromethyl or trifluoromethylthio.

9. The compound of claim 8, wherein Z¹ is phenyl; said phenyl mono-, di- or tri-substituted independently with (C₁-C₆)alkyl, (C₁-C₆)alkoxy, trifluoromethyl or trifluoromethylthio,

10. The compound of claim 8 wherein Z¹ is phenyl which is mono-substituted with (C₃-C₇)cycloalkyl, phenyl, phenoxy or benzyl wherein said (C₃-C₇)cycloalkyl, phenyl, phenoxy, or benzyl are mono-, di- or tri-substituted independently with (C₁-C₆)alkyl, (C₁-C₆)alkoxy, trifluoromethyl or trifluoromethylthio.

11. The compound of claim 1, wherein
A is —C(O)OH;
B is (C₁-C₄)alkylene;
D is NH;
E is carbonyl;
Y is 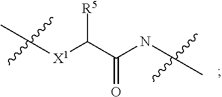
X¹ is 2,3-dihydro-1H-indenyl, indolinyl, tetrahydroquinolinyl or tetrahydronaphthalenyl;
X¹ is optionally mono-substituted with (C₁-C₆)alkyl or trifluoromethyl;
R⁵ is phenyl or (C₃-C₇)cycloalkyl; said phenyl or (C₃-C₇)cycloalkyl mono-substituted with (C₁-C₆)alkyl, phenyl or (C₃-C₇)cycloalkyl; or
Z¹ forms a 5-7 membered heteroaryl or heteroalkyl ring with R⁵, optionally said heteroaryl or heteroalkyl each independently having one or two additional heteroatoms selected independently from nitrogen, sulfur and oxygen having the formula XI;

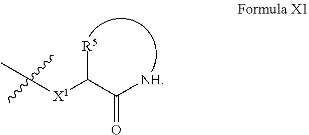

12.-31. (canceled)

32. A pharmaceutical composition comprising a therapeutically effective amount of a compound, or a pharmaceutically acceptable salt thereof, of claim 1 and a pharmaceutically acceptable excipient.

33. A pharmaceutical combination composition comprising: a therapeutically effective amount of a composition comprising:
a first compound, said first compound being a compound of claim 1 or a pharmaceutically acceptable salt of said compound;
a second compound, said second compound being a GLP-1 inhibitor or a GCGR agonist; and
a pharmaceutically acceptable excipient.

34. A pharmaceutical combination composition comprising: a therapeutically effective amount of a composition comprising:
a first compound, said first compound being a compound of claim 1 or a pharmaceutically acceptable salt of said compound;
a second compound, said second compound being a GLP-1 inhibitor; and
a third compound, said third compound being a GCGR agonist; and
a pharmaceutically acceptable excipient.

35.-37. (canceled)

38. A method of treating a GIPR-mediated disease or condition in a subject in need thereof comprising administering to the subject an effective amount of a compound of claim 1.

39. The method of claim 38, wherein the disease is chosen from Type 2 diabetes, elevated glucose levels, elevated insulin levels, dyslipidemia, metabolic syndrome (Syndrome X or insulin resistance syndrome), glucosuria, metabolic acidosis, Type 1 diabetes, obesity, conditions exacerbated by obesity, pancreatitis, cirrhosis and Turner's & Cushing's syndrome.

40. A method of treating a disease or condition selected from Type 2 diabetes, elevated glucose levels, elevated insulin levels, dyslipidemia, metabolic syndrome (Syndrome X or insulin resistance syndrome), glucosuria, metabolic acidosis, Type 1 diabetes and obesity, conditions exacerbated by obesity, pancreatitis, cirrhosis and Turner's & Cushing's syndrome comprising administering to the subject an effective amount of a compound of claim 1.

41. The method of claim 40, wherein the condition is obesity or Type 2 diabetes.

42. (canceled)

43. A method of treating a disease or condition chosen from Type 2 diabetes, elevated glucose levels, elevated insulin levels, dyslipidemia, metabolic syndrome (Syndrome X or insulin resistance syndrome), glucosuria, metabolic acidosis, Type 1 diabetes and obesity, conditions exacerbated by obesity, pancreatitis, cirrhosis and Turner's & Cushing's syndrome comprising administering to the subject an effective amount of a compound of claim 1, or a pharmaceutical composition thereof; and a second compound, said second compound being a GLP-1 inhibitor or a GCGR agonist.

44. (canceled)

45. The method of claim 43, wherein the condition is obesity.
